(19) United States
(12) Patent Application Publication
(10) Pub. No.: US 2014/0199324 A1
(43) Pub. Date: Jul. 17, 2014

(54) EVALUATING PROTEIN EXPRESSION IN PATIENT STRATIFICATION AND OTHER THERAPEUTIC, DIAGNOSTIC AND PROGNOSTIC METHODS FOR CANCER

(71) Applicant: Daniel T. Dransfield, Hanson, MA (US)

(72) Inventor: Daniel T. Dransfield, Hanson, MA (US)

(21) Appl. No.: 14/007,102

(22) PCT Filed: Mar. 23, 2012
(86) PCT No.: PCT/US12/30398

§ 371 Date: Mar. 6, 2014

Related U.S. Application Data
(60) Provisional application No. 61/467,305, filed on Mar. 24, 2011.

(51) Int. Cl. G01N 33/574

(52) U.S. Cl. CPC G01N 33/574
USPC 424/158.1; 435/7.4

(57) ABSTRACT

Provided are compositions, methods and kits for quantifying the expression and/or activity of MMP-14 and other biomarkers of cancer, which may be used diagnostically and prognostically, e.g., in patient stratification and evaluation of appropriate therapeutic regimens.

### CROSS REFERENCE TO RELATED APPLICATIONS

This application claims priority to U.S. Application Ser. No. 61/467,305, filed on Mar. 24, 2011. The disclosure of the prior application is considered part of (and is incorporated by reference in) the disclosure of this application.

### BACKGROUND

The membrane type (MT)-matrix metalloproteinases (MMPs) constitute a sub-group of membrane-anchored MMPs that are major mediators of pericellular proteolysis and physiological activators of pro-MMP-2. MT-MMPs activate the zymogenic form of MMP-2 (pro-MMP-2 or pro-gelatinase A). MMP-2, in turn, can activate pro-MMP-9. The MT-MMPs comprise six members of plasma-tethered MMPs, which include four type I transmembrane enzymes (MMP-14, -15, -16, and -24) and two glycosylphosphatidylinositol-anchored enzymes (MMP-17 and -25). In addition to being potent extracellular matrix (ECM)-degrading enzymes, the type I transmembrane MT-MMPs can also initiate a cascade of zymogen activation on the cell surface.

MMPs are extensively studied in cancer and inflammation, and are well-validated in preclinical studies. Existing treatments for cancer, such as chemotherapy and radiotherapy improve the quality of life with no life-prolonging benefits and have significant side effects. Other treatments, such as MMP inhibitors, are being developed and further refined, and may work most effectively in cancers where certain MMPs are being expressed.

Patient stratification allows healthcare providers to assess the risk/benefit ratio of a given treatment and to predict what patients may best respond to a certain course of treatment. In general, the higher the risk of a particular disease, the better the risk/benefit ratio. Relative risk reduction by a given treatment is often similar across subgroups divided by sex, age, blood pressure etc.; however, if the absolute risk is low it may not be worth taking a treatment with serious side effects. Patient stratification is also important in assessing the cost effectiveness of treatment for a given set of patients.

### SUMMARY

Provided are compositions and methods for quantifying the expression or activity of MMP-14, MMP-9, TIMP-1, and/or MMP-2 and other biomarkers of cancer, for example, osteotropic cancer, breast cancer, lung cancer, melanoma, pancreatic cancer, colon cancer or prostate cancer, which may be used diagnostically (e.g., to identify patients who have cancer, or a particular subclass of cancer) and prognostically (e.g., to identify patients who are likely to develop cancer or respond well to a particular therapeutic for treating cancer). Kits for detecting MMP-14 and other biomarkers and for the practice of the methods incorporating such detection are also described herein.

Specifically, in certain embodiments, provided are methods of utilizing expression of and/or expression ratios of any two of MMP-14, MMP-2, TIMP (e.g., TIMP-1), and MMP-9 in tumors and other cancer cells in order to stratify patients and identify those who would benefit from MMP-14 inhibitor treatment. For example, patients possessing tumors which express both MMP-14 and MMP-2 may be candidates for MMP-14 inhibitor treatment, and patients with tumors expressing MMP-14 and not MMP-2 may also benefit from MMP-14 inhibitor treatment. In another example, those patients with a high MMP-14/low MMP-9 expression ratio may benefit from MMP-14 inhibitor treatment. Further, by evaluating expression of MMP-14 and other MMP biomarkers (e.g., in a sample from a patient), patients can be diagnosed and potentially be stratified into groupings with different prognoses or drug responses. In some embodiments, “Low” and “High” refer to the intensity of immunohistochemistry staining for expression of a particular protein, e.g., MMP-14, MMP-9, TIMP (e.g., TIMP-1) or MMP-2 in a carcinoma. For example, staining levels that are substantially the same as background levels of staining or about 10%, about 20%, about 30%, or about 40% greater than background levels of staining can be considered to be low levels; and staining levels that are about 2, about 3, about 4 fold or greater than background levels of staining can be considered to be high levels. As another example, in some embodiments, when the ratio of MMP-14/MMP-9 is >1, there is more MMP-14 expression than MMP-9 expression and is considered to be a favorable indicator of MMP-14 inhibitor (e.g., DX-2400) responsiveness in preclinical models and subjects, e.g., subjects with cancer. In this embodiment, these subjects would benefit from and/or are good candidates for (e.g., would be selected for) treatment with an MMP-14 inhibitor. In some embodiments, when the ratio is <1, MMP-9 expression is higher than MMP-14 expression, and that could be an indication of a non-responsive or low responsive cancer, e.g., in a subject with cancer. In these embodiments, a subject with a ratio of <1 would not be selected for and/or would not benefit from treatment with an MMP-14 inhibitor. Expression levels, e.g., levels of staining can be quantified, e.g., as described herein.

Also provided herein, are methods of utilizing MMP-9 activity, expression and/or expression ratios of MMP-9 to a tissue inhibitor of matrix metalloproteinases (TIMP (e.g., TIMP-1)) for use in determining whether a subject with cancer would be a good candidate for treatment with an MMP-14 inhibitor. Such methods are based, in part, on the discovery that the presence of MMP-9 activity can counteract the effects of inhibiting MMP-14 (e.g., using DX-2400). Thus, individuals having low or absent MMP-9 expression or activity will respond to MMP-14 inhibitory strategies. The expression of MMP-9 can be expressed as a ratio to the expression of tissue inhibitors of matrix metalloproteinases (TIMPs), which provides an indication of MMP-9 activity in the sample. Therefore, in some embodiments, the expressional ratio of MMP-9/TIMP (e.g., TIMP-1) is used to determine whether a subject having cancer is a good candidate for treatment with an MMP-14 inhibitor. For example, in some embodiments, when the ratio of MMP-9/TIMP (e.g., TIMP-1) is >1, there is more MMP-9 expression than TIMP (e.g., TIMP-1) expression indicating that a subject is likely to be non-responsive to treatment with an MMP-14 inhibitor such as DX-2400. Alternatively, an MMP-9/TIMP ratio less than or equal to 1 indicates that there is less MMP-9 activity and that a subject with cancer would benefit from and/or is a good candidate for (e.g., would be selected for) treatment with an MMP-14 inhibitor.

Also provided herein, in other embodiments, are methods of utilizing MMP-2 activity, expression and/or expression ratios for determining whether a subject with cancer will likely respond to treatment with an MMP-14 inhibitor. These embodiments are based, in part, on the discovery that high MMP-2 expression and/or activity is indicative that a subject will respond to MMP-14 inhibition in the treatment of cancer. In some embodiments, measurements of MMP-2 expression, activity and/or expression ratios are used to determine if a subject having skin cancer, gastric cancer, esophageal cancer or pancreatic cancer would respond to treatment comprising an MMP-14 inhibitor. In some embodiments, an expression ratio of MMP-2 to another protein, e.g., MMP-14, MMP-9 or TIMP (e.g., TIMP-1), can be used to determine if MMP-2 expression and/or activity is high.

Also provided herein, in other embodiments, are methods of selecting subjects having cancer and a mutation associated with elevated MMP-2 levels and/or activity as likely responders to treatment with an MMP-14 inhibitor. For example, the presence of a mutation, e.g., a germline mutation, in the cyclin-dependent kinase inhibitor 2A (CDKN2A) gene or a protein encoded by that gene indicates that a subject will respond to MMP-14 inhibition in the treatment of cancer. In some embodiments, a mutation, e.g., a germline mutation, in the cyclin-dependent kinase inhibitor 2A (CDKN2A) gene or a protein encoded by that gene is used to determine if a subject having skin cancer, gastric cancer, esophageal cancer or pancreatic cancer would respond to treatment comprising an MMP-14 inhibitor.

Also provided herein are methods of treating cancer in a subject, which includes selecting a subject identified as a likely responder, and administering an MMP-14 inhibitor to the subject. The disclosure also relates to methods of treating cancer in a subject that include selecting a subject identified as a likely non responder to an MMP-14 inhibitor, and administering a therapeutic drug other than an MMP-14 inhibitor to the subject.

Compositions and kits for the practice of these methods are also described herein. These embodiments of the present invention, other embodiments, and their features and characteristics will be apparent from the description, drawings, and claims that follow.

### BRIEF DESCRIPTION OF THE DRAWINGS

FIG. 1 illustrates the relative expression levels of various MMPs, including MMP-14 and MMP-2, in different cancer cell lines. TGI: Tumor Growth Inhibition.

FIGS. 2 and 3 illustrate the effect of DX-2400 on tumor progression in xenograft animal models created using the cancer cell lines of FIG. 1.

FIG. 4 illustrates the effect of DX-2400 on metastasis incidence in xenograft animal models created using the cancer cell lines of FIG. 1.

FIGS. 5A, 5B, 5C show the MMP-14 expression levels in selected cell lines by Western blot (WB) analysis (FIG. 5A); and the effect of a MMP-14 antibody (DX-2400) on MMP-14 positive (FIG. 5B) and MMP-14 negative (FIG. 5C) tumors.

FIG. 6 is a schematic representation of embodiments of the patient stratification methods.

### DETAILED DESCRIPTION

For convenience, before further description of the present invention, certain terms employed in the specification, examples and appended claims are defined here.

The singular forms “a”, “an”, and “the” include plural references unless the context clearly dictates otherwise.

The term “agonist”, as used herein, is meant to refer to an agent that mimics or up-regulates (e.g., potentiates or supplements) the bioactivity of a protein. An agonist can be a wild-type protein or derivative thereof having at least one bioactivity of the wild-type protein. An agonist can also be a compound that upregulates expression of a gene or which increases at least one bioactivity of a protein. An agonist can also be a compound which increases the interaction of a polypeptide with another molecule, e.g., a target peptide or nucleic acid.

“Antagonist” as used herein is meant to refer to an agent that downregulates (e.g., suppresses or inhibits) at least one bioactivity of a protein. An antagonist can be a compound which inhibits or decreases the interaction between a protein and another molecule, e.g., a target peptide or enzyme substrate. An antagonist can also be a compound that downregulates expression of a gene or which reduces the amount of expressed protein present.

The term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and domain antibodies (dAb) fragments (de Wildt et al., Eur J Immunol. 1996; 26(3):629-39)) as well as complete antibodies. An antibody can have the structural features of IgA, IgG, IgE, IgD, IgM (as well as subtypes thereof). Antibodies may be from any source, but primate (human and non-human primate) and primatized are preferred.

The VH and VL regions can be further subdivided into regions of hypervariability, termed “complementarity determining regions” (“CDR”), interspersed with regions that are more conserved, termed “framework regions” (“FR”). The extent of the framework regions and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, see also www.hgmp.mrc.ac.uk). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. In IgGs, the heavy chain constant region includes three immunoglobulin domains, CH1, CH2 and CH3. The light chain constant region includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The light chains of the immunoglobulin may be of types kappa or lambda. In one embodiment, the antibody is glycosylated. An antibody can be functional for antibody-dependent cytotoxicity and/or complement-mediated cytotoxicity.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and LC CDR3. Each of the light chain CDRs can be human. HC CDR3 can be human. One or more of the framework regions can be human, e.g., FR1, FR2, FR3, and FR4 of the HC or LC. For example, the Fc region can be human. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. In one embodiment, the framework (FR) residues of a selected Fab can be converted to the amino-acid type of the corresponding residue in the most similar primate germline gene, especially the human germline gene. One or more of the constant regions can be human or effectively human. For example, at least 70, 75, 80, 85, 90, 92, 95, 98, or 100% of an immunoglobulin variable domain, the constant region, the constant domains (CH1, CH2, CH3, CL1), or the entire antibody can be human or effectively human.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the many immunoglobulin variable region genes. Full-length immunoglobulin “light chains” (about 25 KDa or about 214 amino acids) are encoded by a variable region gene at the NH2-terminus (about 110 amino acids) and a kappa or lambda constant region gene at the COOH— terminus. Full-length immunoglobulin “heavy chains” (about 50 KDa or about 446 amino acids), are similarly encoded by a variable region gene (about 116 amino acids) and one of the other aforementioned constant region genes, e.g., gamma (encoding about 330 amino acids). The length of human HC varies considerably because HC CDR3 varies from about 3 amino-acid residues to over 35 amino-acid residues.

The term “binding” refers to an association, which may be a stable association, between two molecules, e.g., between a polypeptide of the invention and a binding partner, due to, for example, electrostatic, hydrophobic, ionic and/or hydrogen-bond interactions under physiological conditions.

The term “binding protein” refers to a protein or polypeptide that can interact with a target molecule. This term is used interchangeably with “ligand.” An “MMP-14 binding protein” refers to a protein that can interact with MMP-14, and includes, in particular, proteins that preferentially interact with and/or inhibit MMP-14. For example, the MMP-14 binding protein may be an antibody.

“Biological activity” or “bioactivity” or “activity” or “biological function”, which are used interchangeably, refer to an effector or antigenic function that is directly or indirectly performed by a polypeptide (whether in its native or denatured conformation), or by any subsequence thereof. Biological activities include binding to polypeptides, binding to other proteins or molecules, activity as a DNA binding protein, as a transcription regulator, ability to bind damaged DNA, etc. A bioactivity may be modulated by directly affecting the subject polypeptide. Alternatively, a bioactivity may be altered by modulating the level of the polypeptide, such as by modulating expression of the corresponding gene.

The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

The term “cancer” is meant to refer to an abnormal cell or cells, or a mass of tissue. The growth of these cells or tissues exceeds and is uncoordinated with that of the normal tissues or cells, and persists in the same excessive manner after cessation of the stimuli which evoked the change. These neoplastic tissues or cells show a lack of structural organization and coordination relative to normal tissues or cells which may result in a mass of tissues or cells which can be either benign or malignant. As used herein, cancer includes any neoplasm. This includes, but is not limited to, melanoma, adenocarcinoma, malignant glioma, prostate cancer, kidney cancer, bladder cancer, pancreatic cancer, thyroid cancer, lung cancer, colon cancer, rectal cancer, brain cancer, liver cancer, breast cancer, ovarian cancer, bone cancer, and the like.

A “combinatorial library” or “library” is a plurality of compounds, which may be termed “members,” synthesized or otherwise prepared from one or more starting materials by employing either the same or different reactants or reaction conditions at each reaction in the library. In general, the members of any library show at least some structural diversity, which often results in chemical diversity. A library may have anywhere from two different members to about 10⁸ members or more. In certain embodiments, libraries of the present invention have more than about 12, 50 and 90 members. In certain embodiments of the present invention, the starting materials and certain of the reactants are the same, and chemical diversity in such libraries is achieved by varying at least one of the reactants or reaction conditions during the preparation of the library. Combinatorial libraries of the present invention may be prepared in solution or on the solid phase.

The term “diagnosing” includes prognosing and staging a disease or disorder.

“Gene” or “recombinant gene” refers to a nucleic acid molecule comprising an open reading frame and including at least one exon and (optionally) an intron sequence. “Intron” refers to a DNA sequence present in a given gene which is spliced out during mRNA maturation.

The terms “label” or “labeled” refer to incorporation or attachment, optionally covalently or non-covalently, of a detectable marker into a molecule, such as a polypeptide and especially an antibody. Various methods of labeling polypeptides are known in the art and may be used. Examples of labels for polypeptides include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for secondary antibodies, metal binding domains, epitope tags). Examples and use of such labels are described in more detail below. In some embodiments, labels are attached by spacer arms of various lengths to reduce potential steric hindrance. Particular examples of labels which may be used under the invention include fluorescein, rhodamine, dansyl, umbelliferone, Texas red, luminol, NADPH, alpha-galactosidase, beta-galactosidase and horseradish peroxidase.

The “level of expression of a gene in a cell” or “gene expression level” refers to the level of mRNA, as well as pre-mRNA nascent transcript(s), transcript processing intermediates, mature mRNA(s) and degradation products, encoded by the gene in the cell.

The term “modulation”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity or process. In certain instances, such regulation may be contingent on the occurrence of a specific event, such as activation of a signal transduction pathway, and/or may be manifest only in particular cell types.

The term “modulator” refers to a polypeptide, nucleic acid, macromolecule, complex, molecule, small molecule, compound, species or the like (naturally-occurring or non-naturally-occurring), or an extract made from biological materials such as bacteria, plants, fungi, or animal cells or tissues, that may be capable of causing modulation. Modulators may be evaluated for potential activity as inhibitors or activators (directly or indirectly) of a functional property, biological activity or process, or combination of them, (e.g., agonist, partial antagonist, partial agonist, inverse agonist, antagonist, anti-microbial agents, inhibitors of microbial infection or proliferation, and the like) by inclusion in assays. In such assays, many modulators may be screened at one time. The activity of a modulator may be known, unknown or partially known.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

The term “osteotropic cancer” refers to metastatic cancer of the bone, i.e., a secondary cancer present in bone that originates from a primary cancer, such as that of the breast, lung, or prostate.

A “patient”, “subject” or “host” to be treated by the subject method may mean either a human or non-human animal.

“Protein”, “polypeptide” and “peptide” are used interchangeably herein when referring to a chain of amino acids prepared by protein synthesis techniques or to a gene product, e.g., as may be encoded by a coding sequence. By “gene product” it is meant a molecule that is produced as a result of transcription of a gene. Gene products include RNA molecules transcribed from a gene, as well as proteins translated from such transcripts.

“Recombinant protein”, “heterologous protein” and “exogenous protein” are used interchangeably to refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the invention to identify compounds that modulate a bioactivity.

“Stage classification” or “staging” is generally, classification of cancer by progression observable by the naked eye, and TNM classification (tumor-node-metastasis staging) is widely used internationally. The “stage classification” used in the present invention corresponds to the TNM classification (“Rinsho, Byori, Genpatsusei Kangan Toriatsukaikiyaku (Clinical and Pathological Codes for Handling Primary Liver Cancer)”: 22p. Nihon Kangangaku Kenkyukai (Liver Cancer Study Group of Japan) edition (3rd revised edition), Kanehara Shuppan, 1992).

“Therapeutic agent” or “therapeutic” refers to an agent capable of having a desired biological effect on a host. Chemotherapeutic and genotoxic agents are examples of therapeutic agents that are generally known to be chemical in origin, as opposed to biological, or cause a therapeutic effect by a particular mechanism of action, respectively. Examples of therapeutic agents of biological origin include growth factors, hormones, and cytokines. A variety of therapeutic agents are known in the art and may be identified by their effects. Certain therapeutic agents are capable of regulating red cell proliferation and differentiation. Examples include chemotherapeutic nucleotides, drugs, hormones, non-specific (non-antibody) proteins, oligonucleotides (e.g., antisense oligonucleotides that bind to a target nucleic acid sequence (e.g., mRNA sequence)), peptides, and peptidomimetics.

The term “therapeutically effective amount” refers to that amount of a modulator, drug or other molecule which is sufficient to effect treatment when administered to a subject in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “treating” as used herein is intended to encompass curing as well as ameliorating at least one symptom of any condition or disease.

MMP-14, MMP-2 and MMP-9 Biomarkers

Without wishing to be bound by theory, according to preferred embodiments of this disclosure, a cancer to be treated with an MMP-14 inhibitor (e.g., treatment with an MMP-14 binding protein, e.g., DX-2400) expresses MMP-14. In preferred embodiments, the MMP-14 is active. Thus, reagents, e.g., proteins (e.g., antibodies) that specifically bind the active form of MMP-14, e.g., DX-2400 (which binds to the catalytic domain of MMP-14) are suitable reagents to practice the methods described herein. In other embodiments, the total levels of MMP-14 (e.g., inactive and active MMP-14) are measured. As described herein, in a tumor model using cells which do not express MMP-14, the tumor xenograft of such cells did not respond to treatment with an MMP-14 inhibitor, DX-2400. In contrast, a tumor xenograft model using cells that express MMP-14 did respond to treatment with an MMP-14 inhibitor, DX-2400.

According to another preferred embodiment, without being bound by theory, in determining responsiveness to treatment with an MMP-14 inhibitor (e.g., treatment with an MMP-14 binding protein, e.g., DX-2400), the levels of MMP-9 (e.g., active MMP-9) are determined. In preferred embodiments, low to no levels of active MMP-9 indicate that the tumor will be responsive to treatment with an MMP-14 inhibitor. In one embodiment, levels of active MMP-9 are determined by measuring expression levels of MMP-9 and TIMP-1 and calculating an expressional ratio of MMP-9/TIMP (e.g., TIMP-1). The expressional ratio of MMP-9/TIMP (e.g., TIMP-1) can be used as an indirect measure of MMP-9 activity in a sample since it reflects the amount of MMP-9 activity that is not inhibited by TIMP activity. Thus, an expressional ratio of greater than 1 indicates that expression of MMP-9 is greater than expression of the TIMP, signaling that MMP-9 is active in the sample. Conversely, an expression ratio of less than or equal to 1 indicates that TIMP expression is higher than that of MMP-9, indicating that MMP-9 activity is low or absent. Thus, expressional ratios of MMP-9/TIMP≦1 indicate that a subject is a good candidate for treatment with an MMP-14 inhibitor. In some embodiments, the expressional ratio of MMP-9/TIMP will exceed 1 (e.g., +2 or +3) indicating very high levels of MMP-9 activity, which correlates with a poor response to treatment with an MMP-14 inhibitor. In certain embodiments, the TIMP is TIMP-1. It is also contemplated herein that the expressional ratio of MMP-9/TIMP can be used to treat a subject or tumor that has not been tested for expression of MMP-14. In other embodiments, the expressional ratios can be, e.g., MMP-9/MMP-14 or MMP-9/MMP-2.

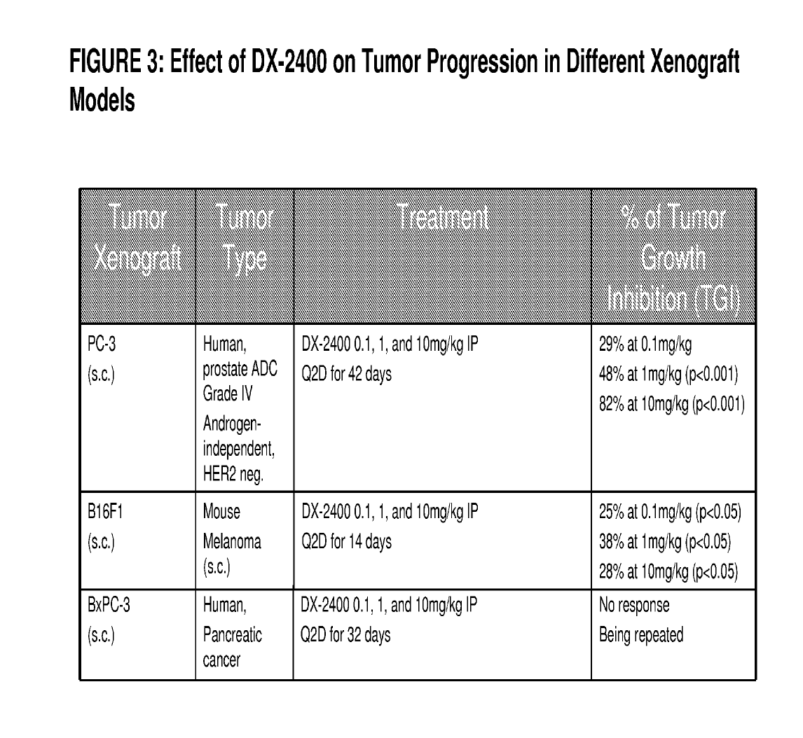

In other embodiments, MMP-9 activity levels can be determined using in situ film zymography or by using an antibody that binds to the active form of MMP-9, e.g., to an active site on MMP-9. Examples of such antibodies include 539A-M0166-F10 and 539A-M0240-B03. As support for this model, experiments were performed using BxPC-3 cells which express active MMP-14 (bind DX-2400) but a tumor of these cells in a xenograft model did not respond in vivo to treatment with an MMP-14 inhibitor, DX-2400 (see FIG. 3). After analyzing the tumor tissue, it was determined that these cells had very high levels of active MMP-9 (data not shown). Thus, in some embodiments, subjects having high levels of active MMP-9 can be selected for treatment with an agent that does not inhibit MMP-14. In other embodiments, subjects having low levels of MMP-9 expression can be selected for treatment with an MMP-14 inhibitor.

The present invention is based at least in part on the observation that certain cancers, particularly osteotropic cancer or bone metastatic cancer cell lines, express MMP-14 and activate proMMP-2, and that MMP-14 inhibitors show enhanced efficacy in cancer cells expressing MMP-14 and/or MMP-2.

According to another embodiment, without being bound by theory, the levels of MMP-2 are assessed to determine responsiveness to treatment with an MMP-14 inhibitor (e.g., treatment with an MMP-14 binding protein, e.g., DX-2400). In preferred embodiments, high levels of MMP-2 indicate that the tumor will be responsive to treatment with an MMP-14 inhibitor. For example, MMP-2 activity levels can be determined using in situ film zymography or by using an antibody that binds to MMP-2, e.g., to an active site on MMP-2. It is also contemplated herein that high levels of MMP-2 can be used to select a subject or tumor for treatment, e.g., with an MMP-14 inhibitor, that has not been tested for expression of MMP-14. In some embodiments, the expression or activity levels of MMP-2 are determined by calculating an expression ratio of MMP-2 to another protein, e.g., MMP-14, MMP-9 and/or TIMP (e.g., TIMP-1).

In other embodiments, subjects having cancer and a mutation associated with elevated MMP-2 levels and/or activity are selected as likely responders to treatment with an MMP-14 inhibitor. For example, the presence of a mutation, e.g., a germline mutation, in the cyclin-dependent kinase inhibitor 2A (CDKN2A) gene or a protein encoded by that gene indicates that a subject will respond to MMP-14 inhibition in the treatment of cancer. In some embodiments, a mutation, e.g., a germline mutation, in the cyclin-dependent kinase inhibitor 2A (CDKN2A) gene or a protein encoded by that gene is used to determine if a subject having skin cancer, gastric cancer, esophageal cancer or pancreatic cancer would respond to treatment comprising an MMP-14 inhibitor. It is also contemplated herein that the presence of a mutation, e.g., a germline mutation, in the cyclin-dependent kinase inhibitor 2A (CDKN2A) gene or a protein encoded by that gene can be used to select a subject or tumor for treatment, e.g., with an MMP-14 inhibitor, that has not been tested for expression of MMP-14.

MMP-14

MMP-14 is encoded by a gene designated as MMP-14, matrix metalloproteinase-14 precursor. Synonyms for MMP-14 include matrix metalloproteinase 14 (membrane-inserted), membrane-type-1 matrix metalloproteinase, membrane-type matrix metalloproteinase 1, MMP-14, MMP-X1, MT1MMP, MT1-MMP, MTMMP1, MT-MMP 1. MT-MMPs have similar structures, including a signal peptide, a prodomain, a catalytic domain, a hinge region, and a hemopexin domain (Wang, et al., 2004, J Biol Chem, 279:51148-55). According to SwissProt entry P50281, the signal sequence of MMP-14 precursor includes amino acid residues 1-20. The pro-peptide includes residues 21-111. Cys93 is annotated as a possible cysteine switch. Residues 112 through 582 make up the mature, active protein. The catalytic domain includes residues 112-317. The hemopexin domains includes residues 318-523. The transmembrane segment comprises residues 542 through 562.

MMP-14 can be shed from cells or found on the surface of cells, tethered by a single transmembrane amino-acid sequence. See, e.g., Osnkowski et al. (2004, J Cell Physiol, 200:2-10).

An exemplary amino acid sequence of human MMP-14 is:

(SEQ ID NO: 1; Genbank Accession No. CAA88372.1)
MSPAPRPPRCLLLPLLTLGTALASLGSAQSSSFSPEAWLQQYGYLPPGDLRTHTQRSPQSLSAAIAAM
QKFYGLQVTGKADADTMKAMRRPRCGVPDKFGAEIKANVRRKRYAIQGLKWQHNEITFCIQNYTPKVG
EYATYEAIRKAFRVWESATPLRFREVPYAYIREGHEKQADIMIFFAEGFHGDSTPFDGEGGFLAHAYF
PGPNIGGDTHFDSAEPWTVRNEDLNGNDIFLVAVHELGHALGLEHSSDPSAIMAPFYQWMDTENFVLP
DDDRRGIQQLYGGESGFPTKMPPQPRTTSRPSVPDKPKNPTYGPNICDGNFDTVAMLRGEMFVFKERW
FWRVRNNQVMDGYPMPIGQFWRGLPASINTAYERKDGKFVFFKGDKHWVFDEASLEPGYPKHIKELGR
GLPTDKIDAALFWMPNGKTYFFRGNKYYRFNEELRAVDSEYPKNIKVWEGIPESPRGSFMGSDEVFTY
FYKGNKYWKFNNQKLKVEPGYPKSALRDWMGCPSGGRPDEGTEEETEVIIIEVDEEGGGAVSAAAVVL
PVLLLLLVLAVGLAVFFFRRHGTPRRLLYCQRSLLDKV.

An exemplary amino acid sequence of mouse MMP-14 is:

SEQ ID NO: 2
MSPAPRPSRSLLLPLLTLGTALASLGWAQGSNFSPEAWLQQYGYLPPGDLRTHTQRSPQSLSAAIAAMQKFYGL
QVTGKADLATMMAMRRPRCGVPDKFGTEIKANVRRKRYAIQGLKWQHNEITFCIQNYTPKVGEYATFEAIRKAF
RVWESATPLRFREVPYAYIREGHEKQADIMILFAEGFHGDSTPFDGEGGFLAHAYFPGPNIGGDTHFDSAEPWT
VQNEDLNGNDIFLVAVHELGHALGLEHSNDPSAIMSPFYQWMDTENFVLPDDDRRGIQQLYGSKSGSPTKMPPQ
PRTTSRPSVPDKPKNPAYGPNICDGNFDTVAMLRGEMFVFKERWFWRVRNNQVMDGYPMPIGQFWRGLPASINT
AYERKDGKFVFFKGDKHWVFDEASLEPGYPKHIKELGRGLPTDKIDAALFWMPNGKTYFFRGNKYYRFNEEFRA
VDSEYPKNIKVWEGIPESPRGSFMGSDEVFTYFYKGNKYWKFNNQKLKVEPGYPKSALRDWMGCPSGRRPDEGT
EEETEVIIIEVDEEGSGAVSAAAVVLPVLLLLLVLAVGLAVFFFRRHGTPKRLLYCQRSLLDKV;
GenBank Accession No. NP_032634.2.

An exemplary MMP-14 protein can consist of or comprise the human or mouse MMP-14 amino acid sequence, a sequence that is 80%, 85%, 90%, 95%, 96%, 97%, 98%, or 99% identical to one of these sequences, or a fragment thereof, e.g., a fragment without the signal sequence or prodomain.

The mRNA sequences of human and murine MMP-14 may be found at GenBank Accession Nos Z48481 and NM_{—}008608, respectively. The sequences of human and mouse MMP-14 mRNAs are as follows:

| SEQ ID NO: 3: human MMP-14 mRNA | |
|---|---|
| 1 | aagttcagtg cctaccgaag acaaaggcgc cccgagggag tggcggtgcg accccagggc |
| 61 | gtgggcccgg ccgcggagcc cacactgccc ggctgacccg gtggtctcgg accatgtctc |
| 121 | ccgccccaag acccccccgt tgtctcctgc tccccctgct cacgctcggc accgcgctcg |
| 181 | cctccctcgg ctcggcccaa agcagcagct tcagccccga agcctggcta cagcaatatg |
| 241 | gctacctgcc tcccggggac ctacgtaccc acacacagcg ctcaccccag tcactctcag |
| 301 | cggccatcgc tgccatgcag aagttttacg gcttgcaagt aacaggcaaa gctgatgcag |
| 361 | acaccatgaa ggccatgagg cgcccccgat gtggtgttcc agacaagttt ggggctgaga |
| 421 | tcaaggccaa tgttcgaagg aagcgctacg ccatccaggg tctcaaatgg caacataatg |
| 481 | aaatcacttt ctgcatccag aattacaccc ccaaggtggg cgagtatgcc acatacgagg |
| 541 | ccattcgcaa ggcgttccgc gtgtgggaga gtgccacacc actgcgcttc cgcgaggtgc |
| 601 | cctatgccta catccgtgag ggccatgaga agcaggccga catcatgatc ttctttgccg |
| 661 | agggcttcca tggcgacagc acgcccttcg atggtgaggg cggcttcctg gcccatgcct |
| 721 | acttcccagg ccccaacatt ggaggagaca cccactttga ctctgccgag ccttggactg |
| 781 | tcaggaatga ggatctgaat ggaaatgaca tcttcctggt ggctgtgcac gagctgggcc |
| 841 | atgccctggg gctcgagcat tccagtgacc cctcggccat catggcaccc ttttaccagt |
| 901 | ggatggacac ggagaatttt gtgctgcccg atgatgaccg ccggggcatc cagcaacttt |
| 961 | atgggggtga gtcagggttc cccaccaaga tgccccctca acccaggact acctcccggc |
| 1021 | cttctgttcc tgataaaccc aaaaacccca cctatgggcc caacatctgt gacgggaact |
| 1081 | ttgacaccgt ggccatgctc cgaggggaga tgtttgtctt caaggagcgc tggttctggc |
| 1141 | gggtgaggaa taaccaagtg atggatggat acccaatgcc cattggccag ttctggcggg |
| 1201 | gcctgcctgc gtccatcaac actgcctacg agaggaagga tggcaaattc gtcttcttca |
| 1261 | aaggagacaa gcattgggtg tttgatgagg cgtccctgga acctggctac cccaagcaca |
| 1321 | ttaaggagct gggccgaggg ctgcctaccg acaagattga tgctgctctc ttctggatgc |
| 1381 | ccaatggaaa gacctacttc ttccgtggaa acaagtacta ccgtttcaac gaagagctca |
| 1441 | gggcagtgga tagcgagtac cccaagaaca tcaaagtctg ggaagggatc cctgagtctc |
| 1501 | ccagagggtc attcatgggc agcgatgaag tcttcactta cttctacaag gggaacaaat |
| 1561 | actggaaatt caacaaccag aagctgaagg tagaaccggg ctaccccaag tcagccctga |
| 1621 | gggactggat gggctgccca tcgggaggcc ggccggatga ggggactgag gaggagacgg |
| 1681 | aggtgatcat cattgaggtg gacgaggagg gcggcggggc ggtgagcgcg gctgccgtgg |
| 1741 | tgctgcccgt gctgctgctg ctcctggtgc tggcggtggg ccttgcagtc ttcttcttca |
| 1801 | gacgccatgg gacccccagg cgactgctct actgccagcg ttccctgctg gacaaggtct |
| 1861 | gacgcccacc gccggcccgc ccactcctac cacaaggact ttgcctctga aggccagtgg |
| 1921 | cagcaggtgg tggtgggtgg gctgctccca tcgtcccgag ccccctcccc gcagcctcct |
| 1981 | tgcttctctc tgtcccctgg ctggcctcct tcaccctgac cgcctccctc cctcctgccc |
| 2041 | cggcattgca tcttccctag ataggtcccc tgagggctga gtgggagggc ggccctttcc |
| 2101 | agcctctgcc cctcagggga accctgtagc tttgtgtctg tccagcccca tctgaatgtg |
| 2161 | ttgggggctc tgcacttgaa ggcaggaccc tcagacctcg ctggtaaagg tcaaatgggg |
| 2221 | tcatctgctc cttttccatc ccctgacata ccttaacctc tgaactctga cctcaggagg |
| 2281 | ctctgggcac tccagccctg aaagccccag gtgtacccaa ttggcagcct ctcactactc |
| 2341 | tttctggcta aaaggaatct aatcttgttg agggtagaga ccctgagaca gtgtgagggg |
| 2401 | gtggggactg ccaagccacc ctaagacctt gggaggaaaa ctcagagagg gtcttcgttg |
| 2461 | ctcagtcagt caagttcctc ggagatctgc ctctgcctca cctaccccag ggaacttcca |
| 2521 | aggaaggagc ctgagccact ggggactaag tgggcagaag aaacccttgg cagccctgtg |
| 2581 | cctctcgaat gttagccttg gatggggctt tcacagttag aagagctgaa accaggggtg |
| 2641 | cagctgtcag gtagggtggg gccggtggga gaggcccggg tcagagccct gggggtgagc |
| 2701 | ctgaaggcca cagagaaaga accttgccca aactcaggca gctggggctg aggcccaaag |
| 2761 | gcagaacagc cagagggggc aggaggggac caaaaaggaa aatgaggacg tgcagcagca |
| 2821 | ttggaaggct ggggccgggc aggccaggcc aagccaagca gggggccaca gggtgggctg |
| 2881 | tggagctctc aggaagggcc ctgaggaagg cacacttgct cctgttggtc cctgtccttg |
| 2941 | ctgcccaggc agcgtggagg ggaagggtag ggcagccaga gaaaggagca gagaaggcac |
| 3001 | acaaacgagg aatgaggggc ttcacgagag gccacagggc ctggctggcc acgctgtccc |
| 3061 | ggcctgctca ccatctcagt gaggggcagg agctggggct cgcttaggct gggtccacgc |
| 3121 | ttccctggtg ccagcacccc tcaagcctgt ctcaccagtg gcctgccctc tcgctccccc |
| 3181 | acccagccca cccattgaag tctccttggg ccaccaaagg tggtggccat ggtaccgggg |
| 3241 | acttgggaga gtgagaccca gtggagggag caagaggaga gggatgtcgg gggggtgggg |
| 3301 | cacggggtag gggaaatggg gtgaacggtg ctggcagttc ggctagattt ctgtcttgtt |
| 3361 | tgtttttttg ttttgtttaa tgtatatttt tattataatt attatatatg aattccaaaa |
| 3421 | aaaaaaaaaa aaaaaaa |
| SEQ ID NO: 4: mouse MMP-14 mRNA | |
| 1 | caaaggagag cagagagggc ttccaactca gttcgccgac taagcagaag aaagatcaaa |
| 61 | aacggaaaag agaagagcaa acagacattt ccaggagcaa ttccctcacc tccaagccga |
| 121 | ccgcgctcta ggaatccaca ttccgttcct ttagaagaca aaggcgcccc aagagaggcg |
| 181 | gcgcgacccc agggcgtggg ccccgccgcg gagcccgcac cgcccggcgc cccgacgccg |
| 241 | gggaccatgt ctcccgcccc tcgaccctcc cgcagcctcc tgctccccct gctcacgctt |
| 301 | ggcacggcgc tcgcctccct cggctgggcc caaggcagca acttcagccc cgaagcctgg |
| 361 | ctgcagcagt atggctacct acctccaggg gacctgcgta cccacacaca acgctcaccc |
| 421 | cagtcactct cagctgccat tgccgccatg caaaagttct atggtttaca agtgacaggc |
| 481 | aaggctgatt tggcaaccat gatggccatg aggcgccctc gctgtggtgt tccggataag |
| 541 | tttgggactg agatcaaggc caatgttcgg aggaagcgct atgccattca gggcctcaag |
| 601 | tggcagcata atgagatcac tttctgcatt cagaattaca cccctaaggt gggcgagtat |
| 661 | gccacattcg aggccattcg gaaggccttc cgagtatggg agagtgccac gccactgcgc |
| 721 | ttccgagaag tgccctatgc ctacatccgg gagggacatg agaagcaggc tgacatcatg |
| 781 | atcttatttg ctgagggttt ccacggcgac agtacaccct ttgatggtga aggagggttc |
| 841 | ctggctcatg cctacttccc aggccccaat attggagggg atacccactt tgattctgcc |
| 901 | gagccctgga ctgtccaaaa tgaggatcta aatgggaatg acatcttctt ggtggctgtg |
| 961 | catgagttgg ggcatgccct aggcctggaa cattctaacg atccctccgc catcatgtcc |
| 1021 | cccttttacc agtggatgga cacagagaac ttcgtgttgc ctgatgacga tcgccgtggc |
| 1081 | atccagcaac tttatggaag caagtcaggg tcacccacaa agatgccccc tcaacccaga |
| 1141 | actacctctc ggccctctgt cccagataag cccaaaaacc ccgcctatgg gcccaacatc |
| 1201 | tgtgacggga actttgacac cgtggccatg ctccgaggag agatgtttgt cttcaaggag |
| 1261 | cgatggttct ggcgggtgag gaataaccaa gtgatggatg gatacccaat gcccattggc |
| 1321 | caattctgga ggggcctgcc tgcatccatc aatactgcct acgaaaggaa ggatggcaaa |
| 1381 | tttgtcttct tcaaaggaga taagcactgg gtgtttgacg aagcctccct ggaacccggg |
| 1441 | taccccaagc acattaagga gcttggccga gggctgccca cggacaagat cgatgcagct |
| 1501 | ctcttctgga tgcccaatgg gaagacctac ttcttccggg gcaataagta ctaccggttc |
| 1561 | aatgaagaat tcagggcagt ggacagcgag taccctaaaa acatcaaagt ctgggaagga |
| 1621 | atccctgaat ctcccagggg gtcattcatg ggcagtgatg aagtcttcac atacttctac |
| 1681 | aagggaaaca aatactggaa gttcaacaac cagaagctga aggtagagcc agggtacccc |
| 1741 | aagtcagctc tgcgggactg gatgggctgc ccttcggggc gccggcccga tgaggggact |
| 1801 | gaggaggaga cagaggtgat catcattgag gtggatgagg agggcagtgg agctgtgagt |
| 1861 | gcggccgccg tggtcctgcc ggtactactg ctgctcctgg tactggcagt gggcctcgct |
| 1921 | gtcttcttct tcagacgcca tgggacgccc aagcgactgc tttactgcca gcgttcgctg |
| 1981 | ctggacaagg tctgaccccc accactggcc cacccgcttc taccacaagg actttgcctc |
| 2041 | tgaaggccag tggctacagg tggtagcagg tgggctgctc tcacccgtcc tgggctccct |
| 2101 | ccctccagcc tcccttctca gtccctaatt ggcctctccc accctcaccc cagcattgct |
| 2161 | tcatccataa gtgggtccct tgagggctga gcagaagacg gtcggcctct ggccctcaag |
| 2221 | ggaatctcac agctcagtgt gtgttcagcc ctagttgaat gttgtcaagg ctcttattga |
| 2281 | aggcaagacc ctctgacctt ataggcaacg gccaaatggg gtcatctgct tcttttccat |
| 2341 | ccccctaact acatacctta aatctctgaa ctctgacctc aggaggctct gggcatatga |
| 2401 | gccctatatg taccaagtgt acctagttgg ctgcctcccg ccactctgac taaaaggaat |
| 2461 | cttaagagtg tacatttgga ggtggaaaga ttgttcagtt taccctaaag actttgataa |
| 2521 | gaaagagaaa gaaagaaaga aagaaagaaa gaaagaaaga aagaaagaaa gaaaaaaaaa |
| 2581 | aaa |

An exemplary MMP-14 gene can consist of or comprise the human or mouse MMP-14 mRNA sequence, a sequence that is 80%, 85%, 90%, 95%, 96%, 97%, 98%, or 99% identical to one of these sequences, or a fragment thereof.

MMP-2

MMP-14 activates pro-MMP-2 causing a cascade of proteolysis that facilitates the mobility and invasiveness of tumor cells (Berno, et al., 2005, Endocr Relat Cancer, 12:393-406; Anilkumar, et al., 2005, Faseb J, 19:1326-8; Itoh and Seiki, 2005, J Cell Physiol; Lopez de Cicco, et al., 2005, Cancer Res, 65:4162-71; El Bedoui, et al., 2005, Cardiovasc Res, 67:317-25; Cao, et al., 2005, Thromb Haemost, 93:770-8; Sato, et al., 2005, Cancer Sci, 96:212-7; Dong, et al., 2005, Am J Pathol, 166:1173-86; Philip, et al., 2004, Glycoconj J, 21:429-41; Guo, et al., 2005, Am J Pathol, 166:877-90; Grossman, 2005, Urol Oncol, 23:222; Gilles, et al., 2001, J Cell Sci, 114:2967-76). Studies propose that this activation process requires both active MT1-MMP and the TIMP-2-bound MT1-MMP (Strongin et al, 1995, J Biol Chem, 270, 5331-5338; Butler et al, 1998, J Biol Chem, 273: 871-80; Kinoshita et al, 1998, J Biol Chem, 273, 16098-103). The TIMP-2 in the latter complex binds, through its C-terminal domain, to the hemopexin domain of pro-MMP-2, which may localize the zymogen close to the active MT1-MMP (Butler et al, 1998, J Biol Chem, 273: 871-80; Kinoshita et al, 1998).

MMP-2 is encoded by a gene designated as MMP-2, matrix metalloproteinase 2 preproprotein. Synonyms for MMP-2 include matrix metalloproteinase 2 (gelatinase A, 72 kD gelatinase, 72 kD type IV collagenase), TBE-1 (as secreted by H-ras oncogene-transformed human bronchial epithelial cells), MMP-II, CLG4, and CLG4A.

An exemplary amino acid sequence of human MMP-2 is:

(SEQ ID NO: 5; Genbank Accession No. NP_004521.1)
MEALMARGAL TGPLRALCLL GCLLSHAAAA PSPIIKFPGD VAPKTDKELA VQYLNTFYGC
PKESCNLFVL KDTLKKMQKF FGLPQTGDLD QNTIETMRKP RCGNPDVANY NFFPRKPKWD
KNQITYRIIG YTPDLDPETV DDAFARAFQV WSDVTPLRFS RIHDGEADIM INFGRWEHGD
GYPFDGKDGL LAHAFAPGTG VGGDSHFDDD ELWTLGEGQV VRVKYGNADG EYCKFPFLFN
GKEYNSCTDT GRSDGFLWCS TTYNFEKDGK YGFCPHEALF TMGGNAEGQP CKFPFRFQGT
SYDSCTTEGR TDGYRWCGTT EDYDRDKKYG FCPETAMSTV GGNSEGAPCV FPFTFLGNKY
ESCTSAGRSD GKMWCATTAN YDDDRKWGFC PDQGYSLFLV AAHEFGHAMG LEHSQDPGAL
MAPIYTYTKN FRLSQDDIKG IQELYGASPD IDLGTGPTPT LGPVTPEICK QDIVFDGIAQ
IRGEIFFFKD RFIWRTVTPR DKPMGPLLVA TFWPELPEKI DAVYEAPQEE KAVFFAGNEY
WIYSASTLER GYPKPLTSLG LPPDVQRVDA AFNWSKNKKT YIFAGDKFWR YNEVKKKMDP
GFPKLIADAW NAIPDNLDAV VDLQGGGHSY FFKGAYYLKL ENQSLKSVKF GSIKSDWLGC.

An exemplary amino acid sequence of murine MMP-2 is:

(SEQ ID NO: 6; Genbank Accession No. NP_032636.1)
MEARVAWGAL AGPLRVLCVL CCLLGRAIAA PSPIIKFPGD VAPKTDKELA VQYLNTFYGC
PKESCNLFVL KDTLKKMQKF FGLPQTGDLD QNTIETMRKP RCGNPDVANY NFFPRKPKWD
KNQITYRIIG YTPDLDPETV DDAFARALKV WSDVTPLRFS RIHDGEADIM INFGRWEHGD
GYPFDGKDGL LAHAFAPGTG VGGDSHFDDD ELWTLGEGQV VRVKYGNADG EYCKFPFLFN
GREYSSCTDT GRSDGFLWCS TTYNFEKDGK YGFCPHEALF TMGGNADGQP CKFPFRFQGT
SYNSCTTEGR TDGYRWCGTT EDYDRDKKYG FCPETAMSTV GGNSEGAPCV FPFTFLGNKY
ESCTSAGRND GKVWCATTTN YDDDRKWGFC PDQGYSLFLV AAHEFGHAMG LEHSQDPGAL
MAPIYTYTKN FRLSHDDIKG IQELYGPSPD ADTDTGTGPT PTLGPVTPEI CKQDIVFDGI
AQIRGEIFFF KDRFIWRTVT PRDKPTGPLL VATFWPELPE KIDAVYEAPQ EEKAVFFAGN
EYWVYSASTL ERGYPKPLTS LGLPPDVQQV DAAFNWSKNK KTYIFAGDKF WRYNEVKKKM
DPGFPKLIAD SWNAIPDNLD AVVDLQGGGH SYFFKGAYYL KLENQSLKSV KFGSIKSDWL
GC.

An exemplary MMP-2 protein can consist of or comprise the human or mouse MMP-2 amino acid sequence, a sequence that is 80%, 85%, 90%, 95%, 96%, 97%, 98%, or 99% identical to one of these sequences, or a fragment thereof, e.g., a fragment without the signal sequence or prodomain.

The mRNA sequences of human and murine MMP-2 may be found at GenBank Accession Nos NM_{—}004530 and NM_{—}008610, respectively. The sequences of human and mouse MMP-2 mRNAs are as follows:

| SEQ ID NO: 7: human MMP-2 mRNA | |
|---|---|
| 1 | gcggctgccc tcccttgttt ccgctgcatc cagacttcct caggcggtgg ctggaggctg |
| 61 | cgcatctggg gctttaaaca tacaaaggga ttgccaggac ctgcggcggc ggcggcggcg |
| 121 | gcgggggctg gggcgcgggg gccggaccat gagccgctga gccgggcaaa ccccaggcca |
| 181 | ccgagccagc ggaccctcgg agcgcagccc tgcgccgcgg agcaggctcc aaccaggcgg |
| 241 | cgaggcggcc acacgcaccg agccagcgac ccccgggcga cgcgcggggc cagggagcgc |
| 301 | tacgatggag gcgctaatgg cccggggcgc gctcacgggt cccctgaggg cgctctgtct |
| 361 | cctgggctgc ctgctgagcc acgccgccgc cgcgccgtcg cccatcatca agttccccgg |
| 421 | cgatgtcgcc cccaaaacgg acaaagagtt ggcagtgcaa tacctgaaca ccttctatgg |
| 481 | ctgccccaag gagagctgca acctgtttgt gctgaaggac acactaaaga agatgcagaa |
| 541 | gttctttgga ctgccccaga caggtgatct tgaccagaat accatcgaga ccatgcggaa |
| 601 | gccacgctgc ggcaacccag atgtggccaa ctacaacttc ttccctcgca agcccaagtg |
| 661 | ggacaagaac cagatcacat acaggatcat tggctacaca cctgatctgg acccagagac |
| 721 | agtggatgat gcctttgctc gtgccttcca agtctggagc gatgtgaccc cactgcggtt |
| 781 | ttctcgaatc catgatggag aggcagacat catgatcaac tttggccgct gggagcatgg |
| 841 | cgatggatac ccctttgacg gtaaggacgg actcctggct catgccttcg ccccaggcac |
| 901 | tggtgttggg ggagactccc attttgatga cgatgagcta tggaccttgg gagaaggcca |
| 961 | agtggtccgt gtgaagtatg ggaacgccga tggggagtac tgcaagttcc ccttcttgtt |
| 1021 | caatggcaag gagtacaaca gctgcactga taccggccgc agcgatggct tcctctggtg |
| 1081 | ctccaccacc tacaactttg agaaggatgg caagtacggc ttctgtcccc atgaagccct |
| 1141 | gttcaccatg ggcggcaacg ctgaaggaca gccctgcaag tttccattcc gcttccaggg |
| 1201 | cacatcctat gacagctgca ccactgaggg ccgcacggat ggctaccgct ggtgcggcac |
| 1261 | cactgaggac tacgaccgcg acaagaagta tggcttctgc cctgagaccg ccatgtccac |
| 1321 | tgttggtggg aactcagaag gtgccccctg tgtcttcccc ttcactttcc tgggcaacaa |
| 1381 | atatgagagc tgcaccagcg ccggccgcag tgacggaaag atgtggtgtg cgaccacagc |
| 1441 | caactacgat gatgaccgca agtggggctt ctgccctgac caagggtaca gcctgttcct |
| 1501 | cgtggcagcc cacgagtttg gccacgccat ggggctggag cactcccaag accctggggc |
| 1561 | cctgatggca cccatttaca cctacaccaa gaacttccgt ctgtcccagg atgacatcaa |
| 1621 | gggcattcag gagctctatg gggcctctcc tgacattgac cttggcaccg gccccacccc |
| 1681 | cacgctgggc cctgtcactc ctgagatctg caaacaggac attgtatttg atggcatcgc |
| 1741 | tcagatccgt ggtgagatct tcttcttcaa ggaccggttc atttggcgga ctgtgacgcc |
| 1801 | acgtgacaag cccatggggc ccctgctggt ggccacattc tggcctgagc tcccggaaaa |
| 1861 | gattgatgcg gtatacgagg ccccacagga ggagaaggct gtgttctttg cagggaatga |
| 1921 | atactggatc tactcagcca gcaccctgga gcgagggtac cccaagccac tgaccagcct |
| 1981 | gggactgccc cctgatgtcc agcgagtgga tgccgccttt aactggagca aaaacaagaa |
| 2041 | gacatacatc tttgctggag acaaattctg gagatacaat gaggtgaaga agaaaatgga |
| 2101 | tcctggcttc cccaagctca tcgcagatgc ctggaatgcc atccccgata acctggatgc |
| 2161 | cgtcgtggac ctgcagggcg gcggtcacag ctacttcttc aagggtgcct attacctgaa |
| 2221 | gctggagaac caaagtctga agagcgtgaa gtttggaagc atcaaatccg actggctagg |
| 2281 | ctgctgagct ggccctggct cccacaggcc cttcctctcc actgccttcg atacaccggg |
| 2341 | cctggagaac tagagaagga cccggagggg cctggcagcc gtgccttcag ctctacagct |
| 2401 | aatcagcatt ctcactccta cctggtaatt taagattcca gagagtggct cctcccggtg |
| 2461 | cccaagaata gatgctgact gtactcctcc caggcgcccc ttccccctcc aatcccacca |
| 2521 | accctcagag ccacccctaa agagatactt tgatattttc aacgcagccc tgctttgggc |
| 2581 | tgccctggtg ctgccacact tcaggctctt ctcctttcac aaccttctgt ggctcacaga |
| 2641 | acccttggag ccaatggaga ctgtctcaag agggcactgg tggcccgaca gcctggcaca |
| 2701 | gggcagtggg acagggcatg gccaggtggc cactccagac ccctggcttt tcactgctgg |
| 2761 | ctgccttaga acctttctta cattagcagt ttgctttgta tgcactttgt ttttttcttt |
| 2821 | gggtcttgtt ttttttttcc acttagaaat tgcatttcct gacagaagga ctcaggttgt |
| 2881 | ctgaagtcac tgcacagtgc atctcagccc acatagtgat ggttcccctg ttcactctac |
| 2941 | ttagcatgtc cctaccgagt ctcttctcca ctggatggag gaaaaccaag ccgtggcttc |
| 3001 | ccgctcagcc ctccctgccc ctcccttcaa ccattcccca tgggaaatgt caacaagtat |
| 3061 | gaataaagac acctactgag tggccgtgtt tgccatctgt tttagcagag cctagacaag |
| 3121 | ggccacagac ccagccagaa gcggaaactt aaaaagtccg aatctctgct ccctgcaggg |
| 3181 | cacaggtgat ggtgtctgct ggaaaggtca gagcttccaa agtaaacagc aagagaacct |
| 3241 | cagggagagt aagctctagt ccctctgtcc tgtagaaaga gccctgaaga atcagcaatt |
| 3301 | ttgttgcttt attgtggcat ctgttcgagg tttgcttcct ctttaagtct gtttcttcat |
| 3361 | tagcaatcat atcagtttta atgctactac taacaatgaa cagtaacaat aatatccccc |
| 3421 | tcaattaata gagtgctttc tatgtgcaag gcacttttca cgtgtcacct attttaacct |
| 3481 | ttccaaccac ataaataaaa aaggccatta ttagttgaat cttattgatg aagagaaaaa |
| 3541 | aaaaaa |
| SEQ ID NO: 8: mouse MMP-2 mRNA | |
| 1 | ccagccggcc acatctggcg tctgcccgcc cttgtttccg ctgcatccag acttccctgg |
| 61 | tggctggagg ctctgtgtgc atccaggagt ttagatatac aaagggattg ccaggacctg |
| 121 | caagcacccg cggcagtggt gtgtattggg acgtgggacc ccgttatgag ctcctgagcc |
| 181 | ccgagaagca gaggcagtag agtaagggga tcgccgtgca gggcaggcgc cagccgggcg |
| 241 | gaccccaggg cacagccaga gacctcaggg tgacacgcgg agcccgggag cgcaacgatg |
| 301 | gaggcacgag tggcctgggg agcgctggcc ggacctctgc gggttctctg cgtcctgtgc |
| 361 | tgcctgttgg gccgcgccat cgctgcacca tcgcccatca tcaagttccc cggcgatgtc |
| 421 | gcccctaaaa cagacaaaga gttggcagtg caatacctga acactttcta tggctgcccc |
| 481 | aaggagagtt gcaacctctt tgtgctgaaa gataccctca agaagatgca gaagttcttt |
| 541 | gggctgcccc agacaggtga ccttgaccag aacaccatcg agaccatgcg gaagccaaga |
| 601 | tgtggcaacc cagatgtggc caactacaac ttcttccccc gcaagcccaa gtgggacaag |
| 661 | aaccagatca catacaggat cattggttac acacctgacc tggaccctga aaccgtggat |
| 721 | gatgcttttg ctcgggcctt aaaagtatgg agcgacgtca ctccgctgcg cttttctcga |
| 781 | atccatgatg gggaggctga catcatgatc aactttggac gctgggagca tggagatgga |
| 841 | tacccatttg atggcaagga tggactcctg gcacatgcct ttgccccggg cactggtgtt |
| 901 | gggggagatt ctcactttga tgatgatgag ctgtggaccc tgggagaagg acaagtggtc |
| 961 | cgcgtaaagt atgggaacgc tgatggcgag tactgcaagt tccccttcct gttcaacggt |
| 1021 | cgggaataca gcagctgtac agacactggt cgcagtgatg gcttcctctg gtgctccacc |
| 1081 | acatacaact ttgagaagga tggcaagtat ggcttctgcc cccatgaagc cttgtttacc |
| 1141 | atgggtggca atgcagatgg acagccctgc aagttcccgt tccgcttcca gggcacctcc |
| 1201 | tacaacagct gtaccaccga gggccgcacc gatggctacc gctggtgtgg caccaccgag |
| 1261 | gactatgacc gggataagaa gtatggattc tgtcccgaga ccgctatgtc cactgtgggt |
| 1321 | ggaaattcag aaggtgcccc atgtgtcttc cccttcactt tcctgggcaa caagtatgag |
| 1381 | agctgcacca gcgccggccg caacgatggc aaggtgtggt gtgcgaccac aaccaactac |
| 1441 | gatgatgacc ggaagtgggg cttctgtcct gaccaaggat atagcctatt cctcgtggca |
| 1501 | gcccatgagt tcggccatgc catggggctg gaacactctc aggaccctgg agctctgatg |
| 1561 | gccccgatct acacctacac caagaacttc cgattatccc atgatgacat caaggggatc |
| 1621 | caggagctct atgggccctc ccccgatgct gatactgaca ctggtactgg ccccacacca |
| 1681 | acactgggac ctgtcactcc ggagatctgc aaacaggaca ttgtctttga tggcatcgct |
| 1741 | cagatccgtg gtgagatctt cttcttcaag gaccggttta tttggcggac agtgacacca |
| 1801 | cgtgacaagc ccacaggtcc cttgctggtg gccacattct ggcctgagct cccagaaaag |
| 1861 | attgacgctg tgtatgaggc cccacaggag gagaaggctg tgttcttcgc agggaatgag |
| 1921 | tactgggtct attctgctag tactctggag cgaggatacc ccaagccact gaccagcctg |
| 1981 | gggttgcccc ctgatgtcca gcaagtagat gctgccttta actggagtaa gaacaagaag |
| 2041 | acatacatct ttgcaggaga caagttctgg agatacaatg aagtgaagaa gaaaatggac |
| 2101 | cccggtttcc ctaagctcat cgcagactcc tggaatgcca tccctgataa cctggatgcc |
| 2161 | gtcgtggacc tgcagggtgg tggtcatagc tacttcttca agggtgctta ttacctgaag |
| 2221 | ctggagaacc aaagtctcaa gagcgtgaag tttggaagca tcaaatcaga ctggctgggc |
| 2281 | tgctgagctg gccctgttcc cacgggccct atcatcttca tcgctgcaca ccaggtgaag |
| 2341 | gatgtgaagc agcctggcgg ctctgtcctc ctctgtagtt aaccagcctt ctccttcacc |
| 2401 | tggtgacttc agatttaaga gggtggcttc tttttgtgcc caaagaaagg tgctgactgt |
| 2461 | accctcccgg gtgctgcttc tccttcctgc ccaccctagg ggatgcttgg atatttgcaa |
| 2521 | tgcagccctc ctctgggctg ccctggtgct ccactcttct ggttcttcaa catctatgac |
| 2581 | ctttttatgg ctttcagcac tctcagagtt aatagagact ggcttaggag ggcactggtg |
| 2641 | gccctgttaa cagcctggca tggggcagtg gggtacaggt gtgccaaggt ggaaatcaga |
| 2701 | gacacctggt ttcacccttt ctgctgccca gacacctgca ccaccttaac tgttgctttt |
| 2761 | gtatgccctt cgctcgtttc cttcaacctt ttcagttttc cactccactg catttcctgc |
| 2821 | ccaaaggact cgggttgtct gacatcgctg catgatgcat ctcagcccgc ctagtgatgg |
| 2881 | ttcccctcct cactctgtgc agatcatgcc cagtcacttc ctccactgga tggaggagaa |
| 2941 | ccaagtcagt ggcttcctgc tcagccttct tgcttctccc tttaacagtt ccccatggga |
| 3001 | aatggcaaac aagtataaat aaagacaccc attgagtgac aaaaaaaaaa aaaaaaaaaa |
| 3061 | aaaaaaaaaa |

An exemplary MMP-2 gene can consist of or comprise the human or mouse MMP-2 mRNA sequence, a sequence that is 80%, 85%, 90%, 95%, 96%, 97%, 98%, or 99% identical to one of these sequences, or a fragment thereof.

Germline mutations (e.g., CDKN2A mutations) can result in elevations of MMP-2 levels and can be used to identify a class of subjects that would be candidates for MMP-14 inhibitory approaches. Various germline mutations in CDKN2A have been associated with cancer. See, e.g., Laytragoon-Lewin et al. Anticancer Res. 2010 November; 30(11):4643-8 and Goldstein, Human Mutation, Mutations in Brief #718 (2004) Online. A reference sequence for CDKN2A, and various isoforms are provided below.

| CDKN2A- cyclin-dependent kinase inhibitor 2A | |
|---|---|
| 1. Gene: | |
| NG_007485.1 | |
| 1 | cgctcaggga aggcgggtgc gcgcctgcgg ggcggagatg ggcagggggc ggtgcgtggg |
| 61 | tcccagtctg cagttaaggg ggcaggagtg gcgctgctca cctctggtgc caaagggcgg |
| 121 | cgcagcggct gccgagctcg gccctggagg cggcgagaac atggtgcgca ggttcttggt |
| 181 | gaccctccgg attcggcgcg cgtgcggccc gccgcgagtg agggttttcg tggttcacat |
| 241 | cccgcggctc acgggggagt gggcagcgcc aggggcgccc gccgctgtgg ccctcgtgct |
| 301 | gatgctactg aggagccagc gtctagggca gcagccgctt cctagaagac caggtaggaa |
| 361 | aggccctcga aaagtccggg gcgcattcgg cacttgtttt gtttggtgtg atttcgtaaa |
| 421 | cagataattc gtctctagcc caggctagga ggaggaggag ataaccgccg gtggaggctt |
| 481 | ccccattcgg gttacaacga cttagacatg tggttctcgc agtaccattg aacctggacc |
| 541 | tcccttcaca cagcccctca atcgtgggaa actgaggcga acagagcttc taaacccacc |
| 601 | tcagaagtca gtgagtcccg aatatcctgg gtgggaatga ctaagacaca cacacacaca |
| 661 | cacacacaca cacacacaca cacacacaca cagtaggaaa ggtgtatttc aagcacactt |
| 721 | tctttctcct tggggagaat tattgctaac catctaagtt ttctggaggc ggcctttttt |
| 781 | ctccccagcc tcccggcggg gtcaccctct cccaccttcc aggagagtgg aggacccgtg |
| 841 | agatacgggg cacgcaggca gcgacttcct gaaatgctaa caaggatcgt aggatcagtt |
| 901 | actgctgcga ggagcaagca cttgcttctt gggggagttt tgcagccaac agggaaatgg |
| 961 | gctttctttg tgagttagag gtagaggtcc ggcggcctga gtgattgaaa ctgctcggga |
| 1021 | caatgctcgt atgtttagca aacgacagaa ctgtagaact gttcctgaga aatcccaact |
| 1081 | gatagtattt tagtcatctc agacgacagt tagcacagtt taaaaatgag gcctacttct |
| 1141 | tgaaaaacag aatccaaggt agttttgtcc tcacattgac aaatgttgac acagccagtg |
| 1201 | taatttccta taaccaggaa aactgaaaga atatatgtac agttaaaata tgtacaatgc |
| 1261 | taattaaaac ttgtgtaata agtctaaaag taatttaatg aggcttcact tttatgaccg |
| 1321 | tccttgtggt atgcttcgcc aggaatatat agcttcaaaa agcaaaggcc agcggagggg |
| 1381 | taattatttt tttactgcaa tgttaattgt ctctttgaca tggaaatata aacctgttaa |
| 1441 | aactatcagt gtttaattta gtgtctcaat ttctattagc aaaaatttat aatctatagg |
| 1501 | ataaatgcac attttatttt ttacttttca tattatgcaa gttaattttt ttaatttagt |
| 1561 | caaaggagct tataaaggat ttcagggcct gttgctggat ttgattttaa ttcattttga |
| 1621 | aacattgaca agaccctggt tgttgttttt tttaacagtg gtttatccgt atcagcaaaa |
| 1681 | gtttagccac tgtgaccggt aactgtatga atatagttct taatattatt gtctatataa |
| 1741 | aaatatttat tactctagtt aatattattc tatataaaat cattttgttt aaattattaa |
| 1801 | gttgcctctg aaaatctgta gtaacaaagt agaacatgtc aatgtatata aatgccataa |
| 1861 | ttatgtattt tttagtttag gcctataaaa cataacattg tggtgatttt aagttagaga |
| 1921 | aaatatttta tagtatgtta atgtatatgc atgaaatgca aaaatattta aatgataggt |
| 1981 | tcattgaaat agatcatttt ttgttattta ggtataaatc aattttcagg acgtatgtga |
| 2041 | aaagcgcaat cttcaggaag tttctcaaga tagaacacag cttggataga atgtcttgaa |
| 2101 | atatatgcaa ttttccaatt tcatatgtaa aatgatatac ataatataaa atctagcggt |
| 2161 | gttaattata atgatatgta attatatatt tcacattaat atattttatg cccatggcta |
| 2221 | tattgatttg ggaatatata tggatactaa ttatgttagg attcatacaa ttccttgaga |
| 2281 | ggcacaagtg ctaaaaatta cttgtatgaa ttatttaata tcattgcaaa taagatgtta |
| 2341 | ttttaacttt ttttaagttt ctgcaaatat gtttattatg actttttatt tttatatgat |
| 2401 | tggaaataca tatactaaaa ttccacgtta ccagtttctt aaccacagaa acctgaaaaa |
| 2461 | ttgccatagt tgatttgtta cttctacctt ggtgcattta caaaatagtc atatttttat |
| 2521 | tatgaagtta aatattcatt tgtttatagc tacttcagaa ggctcaggtt atttttttct |
| 2581 | ttaatagcac agagtcctct caaggtaagc actgtgcagt tagtataaac cattattccc |
| 2641 | catgtgtaca tgattcacag tttgtattgt gttccaagtg aaccatagcc ctttcagaaa |
| 2701 | tcaagactta tattcatttt acttctttga gtactcttga attttagaaa gtccattatg |
| 2761 | atcctaaggt agcaacaaca tagcctatta ccgtctatga tggtttacag atctattatt |
| 2821 | ccacgttagt tcatcactat caactaccat gatagagtta agctaaacca ttttcccaac |
| 2881 | atatgaaaaa ctcctattac taaagtgata caaatggtat caaaaatact tttttatagc |
| 2941 | aaggttcaac agtgggccca gtgcttttac actttttcaa aagtccttgg agaaacagag |
| 3001 | aaaatctcac ttgccttctg tactaaaaca ttctaggccg aactaaaact gaaacttcat |
| 3061 | agtagaacac tgtaggccag gggtgttcaa tcttttacct tccctgggcc acataggaag |
| 3121 | aagaagaatt gtcttgggac acacattaaa tacactaaca ctaacaatag ctgatgagct |
| 3181 | taaaaaaaaa aaaaaactca tgatacttta agaaagtgta tgaatttgtg ttgggcagca |
| 3241 | ttcaaaacca tcctgggctg catgtgaccc tcgggcccac aggttggaca agcttgccct |
| 3301 | agctcctcca tctgctgcaa agcccagcct gatacaaaaa ccaacgtgat aaaaagtttt |
| 3361 | tgtggtgctt tattttggca gtttaagtta tataaacaat gggtacagtt tcattttcta |
| 3421 | aatataaaat ttttacattg aatatgaatt tttaagacaa attatctgaa ttctgattct |
| 3481 | catataccta actactaata tcttctctat ttgttgccca atgagattaa tccacctctt |
| 3541 | aaacacttca ccatcaagaa aaacaatttt gtattttaaa atgaacccat ccactttcat |
| 3601 | tcagctattt tatattcagg catcatccta aggaaagaaa ggttctgaca aagattaata |
| 3661 | cagatggata agtagtagca agaaatcaaa aactgcataa aattctagca ataaagtgtt |
| 3721 | aaattatggt acagttacat tctggatcat caggtatctg aagaatattt catagactgt |
| 3781 | taaatgattg cattataaag tcaggttttt ttaaagcaag attccaaaca gtaaacagtt |
| 3841 | tctctctctc tctctctctc tctctctctc tctctctctc accaaattag ttataatggt |
| 3901 | ttccgcagga tgagaggggt tgggaaaaag tttggtgata tttattttct tcgtttcact |
| 3961 | tttgagtttt ccaaagtgct atgaccatca tcagtaaaat atacatttcc aaagcctttg |
| 4021 | acacacggta acagtcctac acagtggatg aactaagagc ttctctaccc ttagatgggt |
| 4081 | agggagggag gaaagacaag gaaactgagt tgtttaagtg tcatacacga gaacgtggct |
| 4141 | ttaaggtctg ggaaaacctg cgagggctgt gacgtcagac tgtgaaatgc acgctatgtc |
| 4201 | cattcaccaa gacgttccat tttaaaaccc ataaatccgt agctatacct gtttccaagg |
| 4261 | tgcctcgtgt taggcctctg gtcacagcac ttggcgccct tcttgggatc tcttctctcc |
| 4321 | gcccccacta ccccacccca caagcacact ctagtcccct ccaatcaatt tcaggcaggt |
| 4381 | ctcgccgcct ccggagccac gctgggggtg caagggccct ggacccgaaa gagcgcccgc |
| 4441 | ccggcgacaa gagatgagat gcacgctgct cctccactcc tcagccccca ccatcctcct |
| 4501 | cctggatcct aacttcccca ctctctcaat tcctagagac gctgcggatc ccagaggctt |
| 4561 | aactggcagc tggaacgagg tcctccaaca agaatttaga cgctaggtcc aattatcact |
| 4621 | ccaccgcgcg cactttccgc aggagcgatg tgatccgtta tcataactgc ggacctgggg |
| 4681 | ttccacgtgg aagacgattg ggatttcact ggccgcggtg ggggtgggag cagacagagt |
| 4741 | ctgagtgggg ttagtggact cgagacgaaa ggcaggacat gacagaaggc aactctgggt |
| 4801 | cacctctcca gcttggaact ggctaggcct tgttttggag gggatgggta gatgaaaagt |
| 4861 | gagtcagggt tacccggagg aaccacgggg aaagtgcgct tctgagactc ttgacagcca |
| 4921 | tttcgttccc ttccaagcca gatggagacc caagagtgtt gaaaggccac gacttccctc |
| 4981 | agtttctcca tctgggggtg caggatggta tagagagtgg cccgtagtat ttttccagtg |
| 5041 | acgatgtctc tccattgttt tcttcttata ttgcagcttt ccccatgttt gaaaattttc |
| 5101 | ttttcaaatg aaatcattga ttagaataaa aaaaagtaag tagctattaa aacaagatca |
| 5161 | atttccatga cagtaagcca accgatggag aaaaccttgg gaattaataa atgaaggatt |
| 5221 | tgtttggtag atgataaaag gtccttttaa agggtctgac tcttcctaga aaaacccacc |
| 5281 | aacttgggac cgcaacagat ttaccatatc ctaattcatg ctattttaat gtgtattcag |
| 5341 | caaacccaca tgtgtttaca attgtcgaag ctaccaaatg tcaatagcgt tttttttcta |
| 5401 | tttgttgaat gtgaatctct tgtacgaagc catataaaca gaagaaatta caggaatgat |
| 5461 | tttaaatcac atacaaaacc aatagtattg ctagaggaga gttagtcaag gacggcatta |
| 5521 | tgaagaaagt gagggagaat ttccaaagag cagaacgata gggcttggtg gaccaaagaa |
| 5581 | cgtttccatc taaagggaat ggcaaatact tagagtctct gaacccactg aatcttggac |
| 5641 | tatttaacta atatttgtag ttccagatat agcacagtgc cttgtacata gtggtatttt |
| 5701 | taaaaatata gtgcctcgta gatttttttt caacttttat ttaggaggag agggcacatg |
| 5761 | tgcaggttaa ttacaaaggt atattgcacc atgctgaggt ttcgagtacg actgaatctg |
| 5821 | tcactcaagt agtgagcaca gtacccacag taggtagtat ttcagccctc gctcattccc |
| 5881 | tttctcctcc atctagtagt ccccaatgtc tattgttctc atatttatgt ccaattagca |
| 5941 | tttgtttttt aaaaagggtg gttgaagaaa ttctcagtgc ttgtcagtgt ctctcagtgc |
| 6001 | attcatttaa ttcatgagcc ctggaatgat ggtttcattt gggcagaact ctacaatcaa |
| 6061 | aaagaagtaa taaaagggaa aaaaaagtga aagccatcaa ctacaggatt gaaattccca |
| 6121 | aagcatcaga ggtcctttca aaaaatagta tgttgatttt taatttttat gacttattgg |
| 6181 | ctttgttcat gaaaatataa acatgttatc acaaaggatt ttttaattca actatttctc |
| 6241 | agttttctct ttcaccttca aaataaaata tcataaatta tttaaatggt tgtgaaggca |
| 6301 | gtaggatttt tttaagagag aaaagtttta tagaggttca gaattacatg aacaaagaca |
| 6361 | tgtaatctct taagcaaatt gaaactaata aaatcgtaca atcaaggtaa cgtaaataaa |
| 6421 | aaagcctctg ctttcttaat tgaattatgt gagtaactag aaattttaaa agtatggcaa |
| 6481 | aggttaacaa cagcattatt acctgggctg cctttaaaaa tacatatttc tggggttcac |
| 6541 | gttcagaaaa tttgattcag atttgctgtg ggtcccagaa atctgcattt taaataaaca |
| 6601 | cttgaaggag atactaatac aagtggccca ttgggacaca atttgacaaa tatgaccaat |
| 6661 | tttacttttt aaaccttatt tctgcttctt tatctttgaa ttgaggtcca ggattttagg |
| 6721 | taagatttta agtttagagt cagtttactg gatcccaggg aggagagtct gagtaatcag |
| 6781 | tggaggagtt atttcaccaa atgaaggaga ccctttatta ttatgtgacc ctttgtatga |
| 6841 | attggaaaag aatgtcttgt agataccaca tttttacagt cagaacatag tttgagagaa |
| 6901 | aaaaatataa caagatatat ttgtgtttta aagcttacag aaccagacag aaaatttcca |
| 6961 | cataagctat ataagatacg ttgtcttttt aaaacactat atacacttct ttctgttcgt |
| 7021 | gcaggatgaa tggatctctc tctctctctc tctctctctg tgtgtgtgtg tgtgtgtgtg |
| 7081 | tgtgtgtgtg tgtgttgtaa taaggggttt ctttcatttt atgatccaga ccaggctcgt |
| 7141 | aataaacatg acaacctaaa attatgtaaa aaagaaaaat caaagcacaa gtgtttcaca |
| 7201 | ggtttaactt atgcttatct aagatcaggg caagattgca ggaaaatgta gccataacag |
| 7261 | aataaagcat ttatggacaa aatgatgggt ctttatgtct ctgtaaaagc acagtgatgg |
| 7321 | ggggggaaat atagatgaaa aatgtaagct aaaaagtaac aattataaga aaaactaaaa |
| 7381 | tatcatgcct ttcaaatgat catttttctg cttttaagct aaaatttgtc taatattaca |
| 7441 | ccagtgactt tgctgatgta ttaggaaaaa gcttgttttg ctttcttttc tcgagtgcca |
| 7501 | ccattttctt gctctcattc tctttcaggc tgccagatca tctgactcag caattgtata |
| 7561 | actctctcac ccaatttaaa gaaacagcag ctgtctagag aacaatgact cccccagttg |
| 7621 | aacatctaat tgttaaatgt ccaacatcgg acactttgaa ttttactcca tgcaatttac |
| 7681 | atgctgaata gttgaagttg aatatattat atttaacatt taatttttaa aagcttattg |
| 7741 | aaactttctt cctaaatcac atggtaaagt tattgttttc ttcaaaaaca attaggagga |
| 7801 | gcttaacaat aataggacac ttcaacttcc attatctaat ttaattatca caatatcctt |
| 7861 | atgttttcaa tgtttcattt tttcattttg tagatctgga gactgaggct cagataggtt |
| 7921 | gcatggccta ccaaaagtca ttgactagta attcatatat agttgaactt ggttgcccat |
| 7981 | ggagtgctat aaatatgtat atggtttcag ttccatctct tttagttaac tattattttg |
| 8041 | aaagtcgctt aacccctttg ggcctctact atactcaagc atcagccgta taagtcacag |
| 8101 | taaatattta ttggttgaaa ggaggttaac atctttcaaa aatttatttt ttgaccaaaa |
| 8161 | taaaaccagt gaaaaattct catatgactg tacatataaa ttacttattc ctaccttaat |
| 8221 | ttaaaagcaa taagtgggat acctattcac cagcacagga accacttgaa gcgtgcagtt |
| 8281 | gaaagattac tttctttagc attcacatga cctgtgagca gattctattt cttttgctta |
| 8341 | ttagctgtca tggtaccaga atgaagtatg agaaactctc agtgctttca tgttctcatc |
| 8401 | tgtaaacctg agaccctatg gtagtcccgt aataagaggt agataaaata gtatgtgtga |
| 8461 | agagtcactg taaactttta cacagtgtac gtttgtcagt tattatagtg cctaattaaa |
| 8521 | ctatgccctt aagaaagcac attagttttt tacagtaaat acctacttca ttataatttt |
| 8581 | tcagtgtagc tagaaatttc taaactccac tttaaaaata tacatatcat aataaaaata |
| 8641 | tatttatgta ttcagactcc tggtatgttc caaggtgtta ggtaaaatca gtgtaaattt |
| 8701 | gcatactttt aaattcacat ctgtacagaa gatctatatg gtggccttta gggtatacct |
| 8761 | ctaagctatt ctagtattca taatcattaa agagatatta agcagtgttt gtgaacccct |
| 8821 | gttttctaag acaggaaatc aaggtagctt tagaaaactg gaaaaaaagt tattagtcta |
| 8881 | tctatctaat aacccagaat aataatttcc aaaggaatca ctgaagataa ctggattttt |
| 8941 | aattccttca gaatggttgt cacagtctga atatctgaat caacagtttt gaccaaaaca |
| 9001 | attttctaaa aattctttag tataaaaaat tatgtgtgtg tgtgtctgtg tgatgaaagg |
| 9061 | aatgataggc agaaacatta ctgtcatcct tacgacattc aaaatgccta ccttggaggg |
| 9121 | tgaccttcag ttatttttat gcaaatgtga agaagttatt tagaagtagg atatcaaaga |
| 9181 | gtaacacaaa atacactaaa tagtatgctt tcttaaggct aaattgactt gggggtttta |
| 9241 | aatcagtaca gagtaaacat acagtatatt ctgttatcat tgcctttttg aaaaattaat |
| 9301 | tatggaagtt atcatcttaa ccgtaacaac acaaaagata aaactctacc ctcaacccag |
| 9361 | agactcaaag gaaaacatga gtggaaatgt taaatctgta tgtgaaaagt gctaaaacat |
| 9421 | gaataggaag cagttactta tttaatcaaa gttgattata tttcatcaag aagttgattc |
| 9481 | ccttgagtgg agttgaatca catatcaggt gaagaatgtg atttggggaa gaatggtcta |
| 9541 | acacaagaaa attttcttgc aatctttaat aatatcagag gggagattgg cttcagaact |
| 9601 | ctcctaagtt caggaaagga cacagaaaat tgaacataac agtaagacta tagagtccca |
| 9661 | agaaagcaag ctacttttaa aggatagttt tttagagggg caaaaggggg acaaccattc |
| 9721 | tccatttgat gagaaaagct tccatgtaga tggtgcccct gaaattagag tatcctaaac |
| 9781 | cagtgttaaa cctatcagtg aaacatgaat attaaacctc cactcccagt agtgaaaacc |
| 9841 | gaatacatta ttatttatct gtgactttca acattatctc agaactctaa cagcacatgc |
| 9901 | gtacatcagc agcataagca gaaatgagat attatatatg cttgtgttag caattaaaaa |
| 9961 | ggacagcata tttgagaggg gaaaatctgt cctatcaaga atgaaaaaga gggaggttag |
| 10021 | gaaaagtagt ttagagaaag taaattttgc aattcctcag ttttaactgt agtttctcca |
| 10081 | ttgtaccttc cacttgaaat gcactccaag cagtggaggt gggtagcaat gaatgcagag |
| 10141 | gaaacactga acacagtgac actctccagt gtcacttctc atgatttaat gaggggtttt |
| 10201 | ttttggaaat tcttctgtca taacatggga aactttgtta caaagaagct gttttttcag |
| 10261 | agggttagaa ttcagaggta gcatcatacc ttttagaaga gaatttgctt gttgaaacca |
| 10321 | cagatacctg ctagaatgta caggaattaa tgaaaaatta ctcaaaagga catttatttt |
| 10381 | gatgacctaa atgaataact tcatagtaaa tgtcatatat attctcaaaa aattaaaaag |
| 10441 | caccatttat tgagagccta ccgtgcaccc ggatttttat atatctgaca ttctttattc |
| 10501 | ctcacagtaa ccttatgggg taaattttat tttccccact ttgtgaggtg aggaaataaa |
| 10561 | ggctcagaaa gtttacataa cttattcaag cccacagagc tggtaaatga gaggtcagtt |
| 10621 | ctatctgagt ttaaagacta ggcttgtccc acttgcatat gtgtcatttc caaaattatg |
| 10681 | attaaggata tggttggcat ttcccgccac ccacattaag tccaattaag tagctgtggc |
| 10741 | catagaaaga atggagaatg gagagaggaa ctgacttcaa cagctacagc aaacatttat |
| 10801 | tagctgagta accatagcta catagttcct caatatgtac cactcctcca ttttgttatc |
| 10861 | tataaatcaa aatggtggct ttttaaaaag cagttttaca atatattcaa gagccttcta |
| 10921 | ccctttgaaa aactgcaata ctatttttag tagcaattag aaacacctta aatatctgac |
| 10981 | aacagggaca tcattaagta aattataact ttttccagtg ggatgtgtta cagctgttaa |
| 11041 | aagtagcatt tatgaagtgt ttttggagaa gtttggaaaa tgctgtaata agttagaaaa |
| 11101 | agctcatttc aaaattgcat aatattcaca atgtaaagat taagcaaaga aaaaggaaga |
| 11161 | agtatttcaa aatgttaata attattgctt tgtgtggggt agtttttcat tttctatgtg |
| 11221 | cagctaattc cttaattatt tttaaatatg tgagctttaa tcaggaaagc aaatcattca |
| 11281 | aaaatgaggg gactgaatta agtgactttc aggggacttt gcgtgtcttt gagttccaaa |
| 11341 | tttctatcac tatgtattac tactgaagaa taatcataga agcacagtag tttctgaaaa |
| 11401 | tggagagtca gtaatcttgg cccaggtttt gcaacttgct ctaaagcaga gtcctcaaag |
| 11461 | aaaaggaagc attgatgagt tgtccacaat gtactggata aattatcatt aggaaaacat |
| 11521 | attgtagtag ggagagtgag gacctctcaa acagaactga gaaccttaag tttgaacttt |
| 11581 | tcttttcctt attacttaag cactctgagc tttttttttt gtctgcatta tgaagaaaga |
| 11641 | ataatactct ctatcccatg ggacagctgt ggaattataa attacacata taaaactgct |
| 11701 | tgatgcttgt cacatagctg ggggttgaaa aaatgatagc cattattttc ttggcaactt |
| 11761 | ttaatgaatt ttttattatc tctatttctt tctgcctatc tcctctaatt atgtttatta |
| 11821 | cttattttgt tcctcaggat gaggtcaatt ctcaatatct gtgctgtaca taatatacat |
| 11881 | atataccaaa tatgtgcata tagtatgtac atacatacat actgtgctaa tcttttagtg |
| 11941 | ttctcagctg atcaaatagc tacaaataga tataagtaat tcgccacaag taatttatca |
| 12001 | acataaaaaa aatttacaaa aaagttaagg aataattgtc tccatgagct gcaaagatcc |
| 12061 | ctcatttcac aagagtacac cctagagata ttttaatagt aaatttctca catagattta |
| 12121 | aaatcacatt tgttttgcac ataatttaga aaagatacct gctatataat aagtaatata |
| 12181 | cttttaagtt tccttcaaaa tattcttggg aagatgataa taggtactgc taattctata |
| 12241 | cccagttaac attttggaaa ctaaggttga aaattgtgac ttaactataa ttatgcatta |
| 12301 | aatctacaac acatcaaaga attttgcatt ttgtactcct tactaagatc cagtttgagt |
| 12361 | aggaagataa attttacagt aattctgaat gagggaagtt ggcacagagt ttctaaaaga |
| 12421 | gtaccttcct tatagcaaat actaaataat tgtgctatat tgaatttaat taaatagaga |
| 12481 | atagtaaaag ggagaaagaa acatccaatg ttttgaaact tctagagatc tactcccagg |
| 12541 | gacacattgt tttttcttag caaatctgtt tggaggtctg ctctactttc tcagaggtct |
| 12601 | ccctttcatg ctgaagctat cttttttcct tgtggaacat aagtaattaa ataccttgca |
| 12661 | attatttacc taagaaagtg tttctttccc gtttaaaatg ctcttaccac ccacattgga |
| 12721 | ctcgattatc agaattttta tccggggcag cttcaggagc actttggcac ttcggggcta |
| 12781 | aaccacaatc tgtttttaca tgtttgtgat tatacccgtt ttgtagatca agacattgaa |
| 12841 | gctagtaaaa aaaaaaaaaa gtcatttttt cagggtaaca aagtaggtgg tagaactagg |
| 12901 | acagggactc taatttcctt acattattgc ttttctaaat taaagggatg catggaatta |
| 12961 | ttcctccatt gcctttgcct tcaaataatt atctattgca cccaacatcc tattctagaa |
| 13021 | ctcatctatg aaggcttaac acagctgtac ctgggagctc cattacaggg catatatctc |
| 13081 | gctctcataa gctacttcct aaggaattct ctttaattat gggagctttt ccagactctg |
| 13141 | aaatcttttt ttcctggtaa cacaagtgtg aggtgtcatt tatcagaatg catcacccca |
| 13201 | gtcttccctc ctcaaatgat tactgtaggc tccactcaag agctcatccc agttcaagac |
| 13261 | caccttcctc ctccagagaa gcaaatatat atatacacgt atatatatat atacacgtat |
| 13321 | atatatatat acacgtatat atatatatac acgtatatat atatatacac gtatatatat |
| 13381 | atatacacgt atatatatac acgtatatat atatatacac gtatatatat atacacgtat |
| 13441 | atatatatat acacgtatat atatatacac gtatatatat atatacacgt atatatatat |
| 13501 | acgtgtatat atatatatac attttttttt tttgagacgg agtctcgctc tgttgcccag |
| 13561 | gctggagtgc agtggcgcga tctcggctca ctgcaagctc cgccccccgg gttcacgcca |
| 13621 | ttctccttcc tcagcctccg gagtagctgg gactacaggt gcccgccacc tcgcctggct |
| 13681 | aattttttgt atctttagta gagatggggt ttcaccgtgt tacctaggat ggtctagatc |
| 13741 | tcctgacctc gtgatccgcc cgcctcggcc tcccaaagtg ctgggattac aggcgtgagc |
| 13801 | caccgcgcct ggcagagaag caaatatatt gatggttgtt accaatacat gctcttgact |
| 13861 | aagaaacctt ctttcttaat taatattgac aactttaagc cgagtgcctg acatatatta |
| 13921 | ggtactcagt tactcttttt caactaaagt tatgaatgat gattctaata aaagtaactt |
| 13981 | atttgtctac tagttttatt atgtttattt aattcattag aaaggccatg gacatagtac |
| 14041 | aaaattcaaa caatataaat catggaatgt gaaaagtaag tcacatgccc atcccagttc |
| 14101 | ttcatttcct tacctcacag gtaacagctt ttcctgtatc tccccagaga tattctatgt |
| 14161 | atattttgtt tttaacacca agctatattt aaaacaatta tctttaataa taatgttaat |
| 14221 | attgaaactg gtaaagaaat atgtgtgtat tatctcacct caagcgtaaa caatagaaca |
| 14281 | agagagagcc cattttgaaa attatggaca atgaatctag aaataatctc aaaagatttt |
| 14341 | gcagtcaaaa aatagttcat tagatacatg agaactgtca cttggtctca gtgtagagct |
| 14401 | attgcctcaa ctccctttat tttcctaaca aaatcatctt gcttatccca tgaaatacgt |
| 14461 | gcatattgcc aatcctacaa tgccgcatca gaaccagaac ccaactctgg aacactacct |
| 14521 | tctcaagtat ctttctgtct ctttatggta atatgttgaa ttaatattca catctattat |
| 14581 | gactagtctt tgatttgtag ggttgctgaa gtagtagcac cactgcaggg ctttctttag |
| 14641 | tttaaagaaa gtaatcaggt gtccctactg tgtcatgatc tccaccctca gctgggttct |
| 14701 | ccagtctggt tttaaagaac aaaacaaaag gcttctctgt ctgagtctta ctcaacccat |
| 14761 | cctctctact cataagaggt attccaaacc tttacgattc tcaaacttcc taaccgacca |
| 14821 | tcttattttc actctgcaaa caagctaacc tcctcattca tagaaggaag tgcctcaact |
| 14881 | tcctccccgt tctgaccttt tctccctccc aaatctatgt atctcttgtg acaaaatcta |
| 14941 | taaccaccgc tgtactttga gttctatttc ttcattattt ttgagggacc tcaagtcctc |
| 15001 | aaaaatatcc tatcttgcct gtgtacttaa cttttctttt attcttttct aactttccct |
| 15061 | tctcttcact tggcacttgc ccttccaggt atatgtgtgc tcaggtctcc tccaccttcc |
| 15121 | atctgcctca cttcatggca tagggccttg aactatcaca accaagctat gaaagagtag |
| 15181 | tcaacgcagt gtccccactt ccttgccatc ccattatcct agtttttctt ttggctctct |
| 15241 | gaggagtcct tcacaggctg gttttcagga ataagtctaa atgaatcact ttcagttttc |
| 15301 | ctaaacttct atgcctttgc acatcctctt acctctgcct agaatatctt tctccttctt |
| 15361 | ttccatcttt aaactctcac atcattcttc aagactggga tcagctctca gcatccggaa |
| 15421 | gcctttgcct actagagaca aatgagaatg agtttggtca ccttttcatt ttcttgtatc |
| 15481 | attctgtgct ttattttgct cttctaagag cgttacatgc ttcatttaat ccctaaacaa |
| 15541 | ctgtttgagg caagtacagt tattatccta atcatgcaaa tgagaaaaca gaggcccaga |
| 15601 | catgttgagt aactttgata aaagttaaag aaccaataag tggaacagtt gaggtttgaa |
| 15661 | ccctggcagt ctgactgtag agatactatg tttgacctac tcccctctgc ccccacccca |
| 15721 | tgtctgccct tagtttctga gcttgttgaa tgaatgaaca ggtggtagtc tttttttgtt |
| 15781 | ataagactga tcagaattaa gacaggttta aatttcacgt gtagaatttt caaaactgca |
| 15841 | aaggcagtgc aaatctaaaa aaagaatggc attctcagga aagaggaaaa gtaagtgtga |
| 15901 | gaataataat aacaataacc aacaaacttt agtaaattta gtaaatgtag taaattttta |
| 15961 | cattaaaagc ttttggacat acattatcat attttatggc cacatgaaat atattataat |
| 16021 | cccattttgc acataggaaa tctgagactg gcataaggag cacagagatc caggacttta |
| 16081 | tattttcatt cttctaggat tttgcacctc aggtcgatat gtatgagtaa actgggagta |
| 16141 | taatgggctc tttaacagaa aaactaggaa agttttccca ctattattaa ttatttacat |
| 16201 | aatatttttt taattttatt attatttata ctttaagttt tagagtacat gtgcacaatg |
| 16261 | tgcaggtttg ttacatatgt atacatgtgc catgttggtg tgctgcaccc atcaactcat |
| 16321 | catttagcat taggtatatc tcctaatgct atccctcccc cctcccccct acataagatt |
| 16381 | tataatggat aatggacttc aatttctaga gcaaaatggc cccacccaag gatgccataa |
| 16441 | tccttccaga gctctactgc aagatatgag atatacatat ctaaaacttg ttcttggtat |
| 16501 | ttccaaagca gtcaactttt acacctgttt ataatgcatc caaatgttgt ttttatatgg |
| 16561 | ttgcatctcc catcttcttc accaatagct atatatattt ttcacaagag ctgaaagagt |
| 16621 | tcttgatgta ggaatccatg gtagagtttc agagaaatcc ctgaattcac tgaaagtttt |
| 16681 | atctagaaat acatgtgcaa gtgaacacat cttttttaaa aaaaatcatt acctactttc |
| 16741 | ttttttgaga agaaggtatt tatttcaaca gactcttgaa ggagcctact cttcccactc |
| 16801 | tcccaccccc attaagaacc actgtaggcc gggcacgatg gctcatgcct gtaatcccag |
| 16861 | cactttggga ggctaaggtg ggtggatcac ctgaggtcag gagttcgaga caagcctagc |
| 16921 | caacatagtg aaaccccgtc tctactaata atacaaaaat tagctgggta tggcagcatg |
| 16981 | tgcctgtaat cccagctact cgggaggctg aggcaggaga attgctcgaa cccgggaggc |
| 17041 | ggaggttgca gtgaaccgag agagatcgtg cggtgccatt tcactccagc ctgggcaaca |
| 17101 | gagcgaaact ccatctcaaa aaaacacaca aaacaaacaa acaaaaagaa agaaccattg |
| 17161 | tattagtgat ggaaatgtgt tccctccctc ccatcctggc aaccactttc ttcctcctcc |
| 17221 | atcataaaat atcttaaact aaactaaaat aattttattt atcgatagtt tgaattttcc |
| 17281 | ctatcattgc tacacagcta attgagaggt accccgagga aaatataaat ggtacagtaa |
| 17341 | tgcattgtag attttaataa catacttgac atcccaaatt gttttcattg gcttcatttt |
| 17401 | aaaaactaca tgttttaaaa tcaagcagac actaaaagta caagatatac tgggtctaca |
| 17461 | aggtttaagt caaccaggga ttgaaatata acttttaaac agagctggat tatccagtag |
| 17521 | gcagattaag catgtgctta aggcatcagc aaagtctgag caatccattt tttaaaacgt |
| 17581 | agtacatgtt tttgataagc ttaaaaagta gtagtcacag gaaaaattag aacttttacc |
| 17641 | tccttgcgct tgttatactc tttagtgctg tttaactttt ctttgtaagt gagggtggtg |
| 17701 | gagggtgccc ataatctttt cagggagtaa gttcttcttg gtctttcttt ctttctttct |
| 17761 | ttcttttttt cttgagacca agtttcgctc ttgtctccca ggctggagtg caatggcgcg |
| 17821 | atctcggctc actgcaacct ccgccttctc ctgggttcaa gcgattctcc tacatcagcc |
| 17881 | tccgagtagc tgggattaca ggcatgcgcc accaagcccc gctaattttg tattttttag |
| 17941 | tagagacagg gtttcgccat gttggtcagg cttgtctcga actcctggcc tcaggtgatc |
| 18001 | cgcctgtctc ggcctcccag aatgctggga ttatagacgt gagccaccgc atccggactt |
| 18061 | tccttttatg taatagtgat aattctatcc aaagcatttt tttttttttt tttgagtcgg |
| 18121 | agtctcattc tgtcacccag gctggagggt ggtggcgcga tctcggctta ctgcaacctc |
| 18181 | tgcctcccgg gttcaagcga ttctcctgcc tcagcctcct gagtagctgg aattacacac |
| 18241 | gtgcgccacc atggccagct aatttttgta tttttagtag agacggggtg tcaccatttt |
| 18301 | ggccaagctg gcctcgaact cctgacctca ggtgatctgc ccgcctcggc ttcccaaagt |
| 18361 | gctgggatta caggtgtgag ccaccgcgtc ctgctccaaa gcattttctt tctatgcctc |
| 18421 | aaaacaagat tgcaagccag tcctcaaagc ggataattca agagctaaca ggtattagct |
| 18481 | taggatgtgt ggcactgttc ttaaggctta tatgtattaa tacatcattt aaactcacaa |
| 18541 | caacccctat aaagcagggg gcactcatat tcccttcccc ctttataatt acgaaaaatg |
| 18601 | caaggtattt tcagtaggaa agagaaatgt gagaagtgtg aaggagacag gacagtattt |
| 18661 | gaagctggtc tttggatcac tgtgcaactc tgcttctaga acactgagca ctttttctgg |
| 18721 | tctaggaatt atgactttga gaatggagtc cgtccttcca atgactccct ccccattttc |
| 18781 | ctatctgcct acaggcagaa ttctcccccg tccgtattaa ataaacctca tcttttcaga |
| 18841 | gtctgctctt ataccaggca atgtacacgt ctgagaaacc cttgccccag acagccgttt |
| 18901 | tacacgcagg aggggaaggg gaggggaagg agagagcagt ccgactctcc aaaaggaatc |
| 18961 | ctttgaacta gggtttctga cttagtgaac cccgcgctcc tgaaaatcaa gggttgaggg |
| 19021 | ggtaggggga cactttctag tcgtacaggt gatttcgatt ctcggtgggg ctctcacaac |
| 19081 | taggaaagaa tagttttgct ttttcttatg attaaaagaa gaagccatac tttccctatg |
| 19141 | acaccaaaca ccccgattca atttggcagt taggaaggtt gtatcgcgga ggaaggaaac |
| 19201 | ggggcggggg cggatttctt tttaacagag tgaacgcact caaacacgcc tttgctggca |
| 19261 | ggcgggggag cgcggctggg agcagggagg ccggagggcg gtgtgggggg caggtgggga |
| 19321 | ggagcccagt cctccttcct tgccaacgct ggctctggcg agggctgctt ccggctggtg |
| 19381 | cccccggggg agacccaacc tggggcgact tcaggggtgc cacattcgct aagtgctcgg |
| 19441 | agttaatagc acctcctccg agcactcgct cacggcgtcc ccttgcctgg aaagataccg |
| 19501 | cggtccctcc agaggatttg agggacaggg tcggaggggg ctcttccgcc agcaccggag |
| 19561 | gaagaaagag gaggggctgg ctggtcacca gagggtgggg cggaccgcgt gcgctcggcg |
| 19621 | gctgcggaga gggggagagc aggcagcggg cggcggggag cagcatggag ccggcggcgg |
| 19681 | ggagcagcat ggagccttcg gctgactggc tggccacggc cgcggcccgg ggtcgggtag |
| 19741 | aggaggtgcg ggcgctgctg gaggcggggg cgctgcccaa cgcaccgaat agttacggtc |
| 19801 | ggaggccgat ccaggtgggt agagggtctg cagcgggagc aggggatggc gggcgactct |
| 19861 | ggaggacgaa gtttgcaggg gaattggaat caggtagcgc ttcgattctc cggaaaaagg |
| 19921 | ggaggcttcc tggggagttt tcagaagggg tttgtaatca cagacctcct cctggcgacg |
| 19981 | ccctgggggc ttgggaagcc aaggaagagg aatgaggagc cacgcgcgta cagatctctc |
| 20041 | gaatgctgag aagatctgaa ggggggaaca tatttgtatt agatggaagt atgctcttta |
| 20101 | tcagatacaa aatttacgaa cgtttgggat aaaaagggag tcttaaagaa atgtaagatg |
| 20161 | tgctgggact acttagcctc caattcacag atacctggat ggagcttatc tttcttacta |
| 20221 | ggagggatta tcagtggaaa tctgtggtgt atgttggaat aaatatcgaa tataaatttt |
| 20281 | gatcgaaatt attcagaagc ggccgggcgc ggtgcctcac gccttgtaat cccttcactt |
| 20341 | tgggagatca aggcgggggg aatcacctga ggtcgggagt tcgagaccag cctggccaac |
| 20401 | aggtgaaacc tcgcctctac taaaaataca aaaagtagcc gggggtggtg gcaggcgcct |
| 20461 | gtaatcccag ctactcggga ggttgaggca ggagaatcgc ttgaacccgg gaggctgagg |
| 20521 | ttgtagtgaa cagcgagatg gagccacttc actccagcct gggtgacaga gtgagacttt |
| 20581 | gtcgaaagaa agaaagagag aaagagagag agaaaaatta ttcagaagca actacatatt |
| 20641 | gtgtttattt ttaactgagt agggcaaata aatatatgtt tgctgtagga acttaggaaa |
| 20701 | taatgagcca cattcatgtg atcattccag aggtaatatg tagttaccat tttgggaata |
| 20761 | tctgctaaca tttttgctct tttactatct ttagcttact tgatatagtt tatttgtgat |
| 20821 | aagagttttc aattcctcat ttttgaacag aggtgtttct cctctcccta ctcctgtttt |
| 20881 | gtgagggagt taggggagga tttaaaagta attaatacat gggtaactta gcatctctaa |
| 20941 | aattttgcca acagcttgaa cccgggagtt tggctttgta gtcctacaat atcttagaag |
| 21001 | agaccttatt tgtttaaaaa caaaaaggaa aaagaaaagt ggatagtttt gacaattttt |
| 21061 | aatggagacg ggagaagaac atgtagaaaa ggggaaatga tgttggctta gaatcctaac |
| 21121 | tacattggtg tttaatatag gaacatttat ttatataaca ttttaaagta ctaaattcat |
| 21181 | attagtatat tatcaaatgg atatattatc aaatgggttt aagcatccta cacattttaa |
| 21241 | ttcaattgat tcattttctt tttgctttgg atttctatca tgatttaaat atttacatat |
| 21301 | gggttacttt ttagattttt catactatga aatataagaa aaacctttaa ggctagtttt |
| 21361 | atgaccaaga cgaaggactt cattgaatac acaaaacaat aaatatactg caacattttg |
| 21421 | tctttctttt tgtagctgca atttggtttg cttatacttt ctctttgtct ctttgaaaac |
| 21481 | tgagtcagtt tcactttctc aggacaggat ttaataacca taatataatt tagtataatt |
| 21541 | ccttgattta ggcaaattat gcaatttgtg tttagtatga aatgtaccta aaaataagta |
| 21601 | actcctcttt aacaccacca tcctcaaact aatataacaa ataacagtta tcctaaaata |
| 21661 | aattgtctac ttccaccatg cagcactcaa attttaaggt tgctatgact gcagacagta |
| 21721 | ttttaaaatt cctctctgga aatggctttg tttccaagat gatttaggaa ccaaagaggt |
| 21781 | gaccatctct tgtttaatga actctcaaat cataaacctg ggaagtgttt tagtttccta |
| 21841 | ctgctgctgt tacaaattat cacaaatgtg ttagctaaaa caaacacaaa attattattt |
| 21901 | tacagttcta gagatcagaa gtcaaaaatg ggtccacaag gtttcattcc ttttggaaac |
| 21961 | tctaaggggc aatctgtttc cttgtctttt ccagcttcta gtgaccatca aattccttgg |
| 22021 | ctcatggtct ctgtattttc tctgtggcct gtgcttccat tcttgtatct tctctctgac |
| 22081 | tgtgaccctc taataaaaac acttggggtt atgttgggcc caccctgaaa attctggata |
| 22141 | atctccctca agaccattaa ttaaatcaca tctgcaaagc ctcttttgcc acataagtta |
| 22201 | atgtattaaa agtttttgag gattaggaca tagacattgg gggtgggggg gcattattca |
| 22261 | gcctaccaca ggaaggaatt ttagggttaa ttaaactagc cttcttattt tatacttgaa |
| 22321 | gaaattgaag ttttggaatt ggagagcatt atgctaaatg aaataagcca aacacagaaa |
| 22381 | gacaaatatc acatgttctc acttatctgt gaaatataaa acaattacat tcttagcagt |
| 22441 | aaagagtaga atggtggtta ctagagctgg ggggtgggag gaatggggag atggtaatca |
| 22501 | agatataaag cctcagttaa gatgggagga ataagtttga ttgttttttt tgagatgtgt |
| 22561 | ttcatagcat gatgaatata gctaaatagt aaatcccaaa tgctctcatt tgacaaaaat |
| 22621 | gtcaaatatt tgagatgatg gataggttac ttagcttgac ttaataattc cccattgtgt |
| 22681 | tcaaagatca taacttcata ttgtaccaca taaatatata caactgtact atcccaatat |
| 22741 | ataattttaa aactaatata atgaaaaaga aattgaagtt caacattccc agaagctaag |
| 22801 | tgtaacttaa aagttttgtg agaatttgtt ttaacaaaca aacaagtttt ctctttttaa |
| 22861 | caattaccac attctgcgct tggatataca gcagtgaaca aaaaaaaaaa aaaaaatctc |
| 22921 | caggcctaac ataatttcag gaagaaattt cagtagttgt atctcagggg aaatacagga |
| 22981 | agttagcctg gagtaaaagt cagtctgtcc ctgccccttt gctattttgc ccgtgcctca |
| 23041 | cagtgctctc tgcctgtgac gacagctccg cagaagttcg gaggatataa tggaattcat |
| 23101 | tgtgtactga agaatggata gagaactcaa gaaggaaatt ggaaactgga agcaaatgta |
| 23161 | ggggtaatta gacacctggg gcttgtgtgg gggtctgctt ggcggtgagg gggctctaca |
| 23221 | caagcttcct ttccgtcatg ccggccccca ccctggctct gaccattctg ttctctctgg |
| 23281 | caggtcatga tgatgggcag cgcccgagtg gcggagctgc tgctgctcca cggcgcggag |
| 23341 | cccaactgcg ccgaccccgc cactctcacc cgacccgtgc acgacgctgc ccgggagggc |
| 23401 | ttcctggaca cgctggtggt gctgcaccgg gccggggcgc ggctggacgt gcgcgatgcc |
| 23461 | tggggccgtc tgcccgtgga cctggctgag gagctgggcc atcgcgatgt cgcacggtac |
| 23521 | ctgcgcgcgg ctgcgggggg caccagaggc agtaaccatg cccgcataga tgccgcggaa |
| 23581 | ggtccctcag gtgaggactg atgatctgag aatttgtacc ctgagagctt ccaaagctca |
| 23641 | gagcattcat tttccagcac agaaagttca gcccgggaga ccagtctccg gtcttgcctc |
| 23701 | agctcacgcg ccaatcggtg ggacggcctg agtctcccta tcgccctgcc ccgccagggc |
| 23761 | ggcaaatggg aaataatccc gaaatggact tgcgcacgtg aaagcccatt ttgtacatta |
| 23821 | tacttcccaa agcataccac cacccaaaca cctaccctct gctagttcaa ggcctagact |
| 23881 | gcggagcaat gaagactcaa gaggctagag gtctagtgcc ccctcttcct ccaaactagg |
| 23941 | gccagttgca tccacttacc aggtctgttt cctcatttgc ataccaagct ggctggacca |
| 24001 | acctcaggat ttccaaaccc aattgtgcgt ggcatcatct ggagatctct cgatctcggc |
| 24061 | tcttctgcac aactcaacta atctgaccct cctcagctaa tctgaccctc cgctttatgc |
| 24121 | ggtagagttt tccagagctg ccccaggggg ttctggggac atcaggacca agacttcgct |
| 24181 | gaccctggca gtctgtgcac cggagttggc tcctttccct cttaaacttg tgcaagagat |
| 24241 | cgctgagcga tgaaggtaga attatggtcc tccttgccct tgcctttcct ttttgtgatc |
| 24301 | tcaaagcatc ctccctccgc ccccattcca tggccccagt tccctactcc cacagctgtc |
| 24361 | tgctgaaact gccaacatta ctcaattgtt tctgggggga ggaacatttt tttttgaaac |
| 24421 | aaaatagata tatgaaacag tacacgggaa ttaacacgaa tatttaaggt aaaacatgac |
| 24481 | cttgaagatt atgaaatcca tcttattttg gcccagaacg ggggcattgg gctccttggg |
| 24541 | ccatagggga gctggggagg acagggtgaa gagttagctc taagccctct gcttggagat |
| 24601 | gctgtaaata cagaacgcaa aatcaccttc gaagttaaag acgcgaagtt cttctttact |
| 24661 | cggcccctcc tcccctcccc cccgccaatt ccctccagtt acagctagca tccaggtccc |
| 24721 | gggaggtgaa gaaggagact tcggctccag ttacagctag catccgggtc ccgatttaga |
| 24781 | aggagctgcc aattacagcg cggttccagg gctgagcaaa aagcctgagg agccaagtgg |
| 24841 | gagagggagt aaaactactg aattgggcca caagcaaatg aataaactga acgactctta |
| 24901 | accaaaccta atatatttaa tccaaacaca caagtctttc atttcttccc tcctcccttc |
| 24961 | cttctcttac tccccaacac cccctcttca agcacaatta attatatggt tagattctac |
| 25021 | tgcgtgatca gccctgttct aggtggtggg cacgccaagg tgaatgagac caaacaagag |
| 25081 | tcttgccctc atggggttta catttggaga cagagtcgat ctgttgccca acctggagtg |
| 25141 | cagtggcgcg atcacagctc actgcagcct caaactccct ggctcaaggg gttctcccac |
| 25201 | ctgagcctcc cgactagctg ggaccacagg tgcacgccac gacgcctggg tttgtttgtt |
| 25261 | tgtttaatag agacgaaggt ctcaccatgt tatctgggct caagcgatca tcccccctcc |
| 25321 | tcctcctaaa gtactgggat tacagtccca agctatcttg cccgacctgg gaaacagacg |
| 25381 | ttaaggaaga taacaatcta ttttcagaga gcgagtttat aaaaccaatg caatgggtaa |
| 25441 | atatgaagtg tgaataggag gagaagctaa agagtggtcg gagaatctaa tgcaagctac |
| 25501 | gggagaaaga aactcaagtg caaatgctgc ctcaggaata aacgtaaaaa gagactttca |
| 25561 | agtgcaaatg ctccctcagg aataaaataa tcttgagact ctcaagtgta aatgctgcct |
| 25621 | cgggagaacc gaacggcgag ctggagccca tacgcaacga gattagagag gaaggcagaa |
| 25681 | gccagagcac atgaataaat gagcatccat tttgtttcag aaatgatcgg aaaccatttg |
| 25741 | tgggtttgta gaagcaggca tgcgtaggga agctacggga ttccgccgag gagcgccaga |
| 25801 | gcctgaggcg ccctttggtt atcgcaagct ggctggctca ctccgcacca ggtgcaaaag |
| 25861 | atgcctgggg atgcgggaag ggaaaggcca catcttcacg ccttcgcgcc tggcattgtg |
| 25921 | agcaaccact gagactcatt atataacact cgttttcttc ttgcaaccct gcgggccgcg |
| 25981 | cggtcgcgct ttctctgccc tccgccgggt ggacctggag cgcttgagcg gtcggcgcgc |
| 26041 | ctggagcagc caggcgggca gtggactagc tgctggacca gggaggtgtg ggagagcggt |
| 26101 | ggcggcgggt acatgcacgt gaagccattg cgagaacttt atccataagt atttcaatgc |
| 26161 | cggtagggac ggcaagagag gagggcggga tgtgccacac atctttgacc tcaggtttct |
| 26221 | aacgcctgtt ttctttctgc cctctgcaga catccccgat tgaaagaacc agagaggctc |
| 26281 | tgagaaacct cgggaaactt agatcatcag tcaccgaagg tcctacaggg ccacaactgc |
| 26341 | ccccgccaca acccaccccg ctttcgtagt tttcatttag aaaatagagc ttttaaaaat |
| 26401 | gtcctgcctt ttaacgtaga tatatgcctt cccccactac cgtaaatgtc catttatatc |
| 26461 | attttttata tattcttata aaaatgtaaa aaagaaaaac accgcttctg ccttttcact |
| 26521 | gtgttggagt tttctggagt gagcactcac gccctaagcg cacattcatg tgggcatttc |
| 26581 | ttgcgagcct cgcagcctcc ggaagctgtc gacttcatga caagcatttt gtgaactagg |
| 26641 | gaagctcagg ggggttactg gcttctcttg agtcacactg ctagcaaatg gcagaaccaa |
| 26701 | agctcaaata aaaataaaat aattttcatt cattcactca |

2.mRNA/protein (Genbank Accession Nos.)

| Isoform | mRNA | protein |
|---|---|---|
| isoform 1 | NM_000077.4 | NP_000068.1 |
| isoform 5 | NM_001195132.1 | NP_001182061.1 |
| isoform 4 | NM_058195.3 | NP_478102.2 |
| p12 | NM_058197.4 | NP_478104.2 |

NM_001195132.1

| | |
|---|---|
| 1 | cgagggctgc ttccggctgg tgcccccggg ggagacccaa cctggggcga cttcaggggt |
| 61 | gccacattcg ctaagtgctc ggagttaata gcacctcctc cgagcactcg ctcacggcgt |
| 121 | ccccttgcct ggaaagatac cgcggtccct ccagaggatt tgagggacag ggtcggaggg |
| 181 | ggctcttccg ccagcaccgg aggaagaaag aggaggggct ggctggtcac cagagggtgg |
| 241 | ggcggaccgc gtgcgctcgg cggctgcgga gagggggaga gcaggcagcg ggcggcgggg |
| 301 | agcagcatgg agccggcggc ggggagcagc atggagcctt cggctgactg gctggccacg |
| 361 | gccgcggccc ggggtcgggt agaggaggtg cgggcgctgc tggaggcggg ggcgctgccc |
| 421 | aacgcaccga atagttacgg tcggaggccg atccaggtca tgatgatggg cagcgcccga |
| 481 | gtggcggagc tgctgctgct ccacggcgcg gagcccaact gcgccgaccc cgccactctc |
| 541 | acccgacccg tgcacgacgc tgcccgggag ggcttcctgg acacgctggt ggtgctgcac |
| 601 | cgggccgggg cgcggctgga cgtgcgcgat gcctggggcc gtctgcccgt ggacctggct |
| 661 | gaggagctgg gccatcgcga tgtcgcacgg tacctgcgcg cggctgcggg gggcaccaga |
| 721 | ggcagtaacc atgcccgcat agatgccgcg gaaggtccct cagaaatgat cggaaaccat |
| 781 | ttgtgggttt gtagaagcag gcatgcgtag ggaagctacg ggattccgcc gaggagcgcc |
| 841 | agagcctgag gcgccctttg gttatcgcaa gctggctggc tcactccgca ccaggtgcaa |
| 901 | aagatgcctg gggatgcggg aagggaaagg ccacatcttc acgccttcgc gcctggcatt |
| 961 | acatccccga ttgaaagaac cagagaggct ctgagaaacc tcgggaaact tagatcatca |
| 1021 | gtcaccgaag gtcctacagg gccacaactg cccccgccac aacccacccc gctttcgtag |
| 1081 | ttttcattta gaaaatagag cttttaaaaa tgtcctgcct tttaacgtag atatatgcct |
| 1141 | tcccccacta ccgtaaatgt ccatttatat cattttttat atattcttat aaaaatgtaa |
| 1201 | aaaagaaaaa caccgcttct gccttttcac tgtgttggag ttttctggag tgagcactca |
| 1261 | cgccctaagc gcacattcat gtgggcattt cttgcgagcc tcgcagcctc cggaagctgt |
| 1321 | cgacttcatg acaagcattt tgtgaactag ggaagctcag gggggttact ggcttctctt |
| 1381 | gagtcacact gctagcaaat ggcagaacca aagctcaaat aaaaataaaa taattttcat |
| 1441 | tcattcactc aaaaaaaaaa aaaa |

//
NM_058197.4

| | |
|---|---|
| 1 | atggagccgg cggcggggag cagcatggag ccttcggctg actggctggc cacggccgcg |
| 61 | gcccggggtc gggtagagga ggtgcgggcg ctgctggagg cgggggcgct gcccaacgca |
| 121 | ccgaatagtt acggtcggag gccgatccag gtgggtagag ggtctgcagc gggagcaggg |
| 181 | gatggcgggc gactctggag gacgaagttt gcaggggaat tggaatcagg tagcgcttcg |
| 241 | attctccgga aaaaggggag gcttcctggg gagttttcag aaggggtttg taatcacaga |
| 301 | cctcctcctg gcgacgccct gggggcttgg gaagccaagg aagaggaatg aggagccacg |
| 361 | cgcgtacaga tctctcgaat gctgagaaga tctgaagggg ggaacatatt tgtattagat |
| 421 | ggaagtcatg atgatgggca gcgcccgagt ggcggagctg ctgctgctcc acggcgcgga |
| 481 | gcccaactgc gccgaccccg ccactctcac ccgacccgtg cacgacgctg cccgggaggg |
| 541 | cttcctggac acgctggtgg tgctgcaccg ggccggggcg cggctggacg tgcgcgatgc |
| 601 | ctggggccgt ctgcccgtgg acctggctga ggagctgggc catcgcgatg tcgcacggta |
| 661 | cctgcgcgcg gctgcggggg gcaccagagg cagtaaccat gcccgcatag atgccgcgga |
| 721 | aggtccctca gacatccccg attgaaagaa ccagagaggc tctgagaaac ctcgggaaac |
| 781 | ttagatcatc agtcaccgaa ggtcctacag ggccacaact gcccccgcca caacccaccc |
| 841 | cgctttcgta gttttcattt agaaaataga gcttttaaaa atgtcctgcc ttttaacgta |
| 901 | gatatatgcc ttcccccact accgtaaatg tccatttata tcatttttta tatattctta |
| 961 | taaaaatgta aaaaagaaaa acaccgcttc tgccttttca ctgtgttgga gttttctgga |
| 1021 | gtgagcactc acgccctaag cgcacattca tgtgggcatt tcttgcgagc ctcgcagcct |
| 1081 | ccggaagctg tcgacttcat gacaagcatt ttgtgaacta gggaagctca ggggggttac |
| 1141 | tggcttctct tgagtcacac tgctagcaaa tggcagaacc aaagctcaaa taaaaataaa |
| 1201 | ataattttca ttcattcact caaaaaaaaa aaaaa |

NM_000077.4

| | |
|---|---|
| 1 | cgagggctgc ttccggctgg tgcccccggg ggagacccaa cctggggcga cttcaggggt |
| 61 | gccacattcg ctaagtgctc ggagttaata gcacctcctc cgagcactcg ctcacggcgt |
| 121 | ccccttgcct ggaaagatac cgcggtccct ccagaggatt tgagggacag ggtcggaggg |
| 181 | ggctcttccg ccagcaccgg aggaagaaag aggaggggct ggctggtcac cagagggtgg |
| 241 | ggcggaccgc gtgcgctcgg cggctgcgga gagggggaga gcaggcagcg ggcggcgggg |
| 301 | agcagcatgg agccggcggc ggggagcagc atggagcctt cggctgactg gctggccacg |
| 361 | gccgcggccc ggggtcgggt agaggaggtg cgggcgctgc tggaggcggg ggcgctgccc |
| 421 | aacgcaccga atagttacgg tcggaggccg atccaggtca tgatgatggg cagcgcccga |
| 481 | gtggcggagc tgctgctgct ccacggcgcg gagcccaact gcgccgaccc cgccactctc |
| 541 | acccgacccg tgcacgacgc tgcccgggag ggcttcctgg acacgctggt ggtgctgcac |
| 601 | cgggccgggg cgcggctgga cgtgcgcgat gcctggggcc gtctgcccgt ggacctggct |
| 661 | gaggagctgg gccatcgcga tgtcgcacgg tacctgcgcg cggctgcggg gggcaccaga |
| 721 | ggcagtaacc atgcccgcat agatgccgcg gaaggtccct cagacatccc cgattgaaag |
| 781 | aaccagagag gctctgagaa acctcgggaa acttagatca tcagtcaccg aaggtcctac |
| 841 | agggccacaa ctgcccccgc cacaacccac cccgctttcg tagttttcat ttagaaaata |
| 901 | gagcttttaa aaatgtcctg ccttttaacg tagatatatg ccttccccca ctaccgtaaa |
| 961 | tgtccattta tatcattttt tatatattct tataaaaatg taaaaaagaa aaacaccgct |
| 1021 | tctgcctttt cactgtgttg gagttttctg gagtgagcac tcacgcccta agcgcacatt |
| 1081 | catgtgggca tttcttgcga gcctcgcagc ctccggaagc tgtcgacttc atgacaagca |
| 1141 | ttttgtgaac tagggaagct caggggggtt actggcttct cttgagtcac actgctagca |
| 1201 | aatggcagaa ccaaagctca aataaaaata aaataatttt cattcattca ctcaaaaaaa |
| 1261 | aaaaaaa |

NM_058195.3

| | |
|---|---|
| 1 | cgctcaggga aggcgggtgc gcgcctgcgg ggcggagatg ggcagggggc ggtgcgtggg |
| 61 | tcccagtctg cagttaaggg ggcaggagtg gcgctgctca cctctggtgc caaagggcgg |
| 121 | cgcagcggct gccgagctcg gccctggagg cggcgagaac atggtgcgca ggttcttggt |
| 181 | gaccctccgg attcggcgcg cgtgcggccc gccgcgagtg agggttttcg tggttcacat |
| 241 | cccgcggctc acgggggagt gggcagcgcc aggggcgccc gccgctgtgg ccctcgtgct |
| 301 | gatgctactg aggagccagc gtctagggca gcagccgctt cctagaagac caggtcatga |
| 361 | tgatgggcag cgcccgagtg gcggagctgc tgctgctcca cggcgcggag cccaactgcg |
| 421 | ccgaccccgc cactctcacc cgacccgtgc acgacgctgc ccgggagggc ttcctggaca |
| 481 | cgctggtggt gctgcaccgg gccggggcgc ggctggacgt gcgcgatgcc tggggccgtc |
| 541 | tgcccgtgga cctggctgag gagctgggcc atcgcgatgt cgcacggtac ctgcgcgcgg |
| 601 | ctgcgggggg caccagaggc agtaaccatg cccgcataga tgccgcggaa ggtccctcag |
| 661 | acatccccga ttgaaagaac cagagaggct ctgagaaacc tcgggaaact tagatcatca |
| 721 | gtcaccgaag gtcctacagg gccacaactg cccccgccac aacccacccc gctttcgtag |
| 781 | ttttcattta gaaaatagag cttttaaaaa tgtcctgcct tttaacgtag atatatgcct |
| 841 | tcccccacta ccgtaaatgt ccatttatat cattttttat atattcttat aaaaatgtaa |
| 901 | aaaagaaaaa caccgcttct gccttttcac tgtgttggag ttttctggag tgagcactca |
| 961 | cgccctaagc gcacattcat gtgggcattt cttgcgagcc tcgcagcctc cggaagctgt |
| 1021 | cgacttcatg acaagcattt tgtgaactag ggaagctcag gggggttact ggcttctctt |
| 1081 | gagtcacact gctagcaaat ggcagaacca aagctcaaat aaaaataaaa taattttcat |
| 1141 | tcattcactc aaaaaaaaaa aaaa |

NP_000068.1

| | |
|---|---|
| 1 | mepaagssme psadwlataa argrveevra lleagalpna pnsygrrpiq vmmmgsarva |
| 61 | ellllhgaep ncadpatltr pvhdaaregf ldtivvlhra garldvrdaw grlpvdlaee |
| 121 | lghrdvaryl raaaggtrgs nharidaaeg psdipd |

NP_001182061.1

| | |
|---|---|
| 1 | mepaagssme psadwlataa argrveevra lleagalpna pnsygrrpiq vmmmgsarva |
| 61 | ellllhgaep ncadpatltr pvhdaaregf ldtivvlhra garldvrdaw grlpvdlaee |
| 121 | lghrdvaryl raaaggtrgs nharidaaeg psemignhlw vcrsrha |

NP_478102.2

| | |
|---|---|
| 1 | mvrrflvtlr irracgppry rvfvvhiprl tgewaapgap aavalvlmll rsqrlgqqpl |
| 61 | prrpghddgq rpsggaaaap rrgaqlrrpr hshptrarrc pgglpghagg aapgrgaagr |
| 121 | arclgpsarg pg |

NP_478104.2

| | |
|---|---|
| 1 | mepaagssme psadwlataa argrveevra lleagalpna pnsygrrpiq vgrgsaagag |
| 61 | dggrlwrtkf agelesgsas ilrkkgrlpg efsegvcnhr pppgdalgaw eakeee |

MMP-9

MMP-9 is a Zn+2 dependent endopeptidase, synthesized and secreted in monomeric form as zymogen. The structure is almost similar to MMP2. The nascent form of the protein shows an N-terminal signal sequence (“pre” domain) that directs the protein to the endoplasmic reticulum. The pre domain is followed by a propeptide-“pro” domain that maintains enzyme-latency until cleaved or disrupted, and a catalytic domain that contains the conserved zinc-binding region. A hemopexin/vitronectin-like domain is also seen, that is connected to the catalytic domain by a hinge or linker region. The hemopexin domain is involved in TIMP (Tissue Inhibitors of Metallo-Proteinases) binding e.g., TIMP-1 & TIMP-3, the binding of certain substrates, membrane activation, and some proteolytic activities. It also shows a series of three head-to-tail cysteine-rich repeats within its catalytic domain. These inserts resemble the collagen-binding type II repeats of fibronectin and are required to bind and cleave collagen and elastin.

Its primary function is degradation of proteins in the extracellular matrix. It proteolytically digests decorin, elastin, fibrillin, laminin, gelatin (denatured collagen), and types IV, V, XI and XVI collagen and also activates growth factors like proTGFb and proTNFa. Physiologically, MMP-9 in coordination with other MMPs, play a role in normal tissue remodeling events such as neurite growth, embryonic development, angiogenesis, ovulation, mammary gland involution and wound healing. MMP-9 with other MMPs is also involved in osteoblastic bone formation and/or inhibits osteoclastic bone resorption.

MMP-9 is encoded by a gene designated as matrix metallopeptidase 9 (gelatinase B, 92 kDa gelatinase, 92 kDa type IV collagenase). Synonyms for MMP-9 include CLG4 (Collagenase Type IV), CLG4B (Collagenase Type IV-B), and GELB (Gelatinase B).

An exemplary amino acid sequence of human MMP-9 is:

| (SEQ ID NO: 9; Genbank Accession No. NP_004985) | |
|---|---|
| 1 | mslwqp1v1v llvlgccfaa prqrqstivl fpgdlrtnit drqlaeeyly rygytrvaem |
| 61 | rgeskslgpa llllqkqls1 petgeldsat lkamrtprcg vpdlgrfqtf egdlkwhhhn |
| 121 | itywicinysedlpravidda farafalwsa vtpltftrvy srdadiviqf gvaehgdgyp |
| 181 | fdgkdgllah afppgpgiqg dahfdddelw slgkgvvvpt rfgnadgaac hfpfifegrs |
| 241 | ysacttdgrs dglpwcstta nydtddrfgf cpserlytqd gnadgkpcqf pfifqgqsys |
| 301 | acttdgrsdg yrwcattany drdklfgfcp tradstvmgg nsagelcvfp ftflgkeyst |
| 361 | ctsegrgdgr lwcattsnfd sdkkwgfcpd qgyslflvaa hefghalgld hssvpealmy |
| 421 | pmyrftegpp lhkddvngir hlygprpepe prppttttpq ptapptvcpt gpptvhpser |
| 481 | ptagptgpps agptgpptag pstattvpls pvddacnvni fdaiaeignq lylfkdgkyw |
| 541 | rfsegrgsrp qgpfliadkw palprkldsv feerlskklf ffsgrqvwvy tgasvlgprr |
| 601 | ldklglgadv aqvtgalrsg rgkmllfsgr rlwrfdvkaq mvdprsasev drmfpgvpld |
| 661 | thdvfqyrek ayfcqdrfyw rvssrselnq vdqvgyvtyd ilqcped |

An exemplary amino acid sequence of murine MMP-9 is:

| (SEQ ID NO: 10; Genbank Accession No. NP_038627) | |
|---|---|
| 1 | mspwqpllla llafgcssaa pygrqptfvv fpkdlktsnl tdtqlaeayl yrygytraaq |
| 61 | mmgekgslrp allmlqkqls lpqtgeldsq tlkairtprc gvpdvgrfqt fkglkwdhhn |
| 121 | itywiqnyse dlprdmidda farafavwge vapltftrvy gpeadiviqf gvaehgdgyp |
| 181 | fdgkdgllah afppgagvqg dahfdddelw slgkgvvipt yygnsngapc hfpftfegrs |
| 241 | ysacttdgrn dgtpwcstta dydkdgkfgf cpserlyteh gngegkpcvf pfifegrsys |
| 301 | acttkgrsdg yrwcattany dqdklygfcp trvdatvvgg nsagelcvfp fvflgkqyss |
| 361 | ctsdgrrdgr lwcattsnfd tdkkwgfcpd qgyslflvaa hefghalgld hssvpealmy |
| 421 | plysylegfp lnkddidgiq ylygrgskpd prppatttte pqptapptmc ptipptaypt |
| 481 | vgptvgptga pspgptssps pgptgapspg ptapptagss easteslspa dnpcnvdvfd |
| 541 | aiaeiggalh ffkdgwywkf lnhrgsplqg pfltartwpa lpatldsafe dpqtkrvfff |
| 601 | sgrqmwvytg ktvlgprsld klglgpevth vsgllprrlg kallfskgrv wrfdlksqkv |
| 661 | dpqsvirvdk efsgvpwnsh difqyqdkay fchgkffwry sfqnevnkvd hevnqvddvg |
| 721 | yvtydllqcp |

An exemplary MMP-9 protein can consist of or comprise the human or mouse MMP-9 amino acid sequence, a sequence that is 80%, 85%, 90%, 95%, 96%, 97%, 98%, or 99% identical to one of these sequences, or a fragment thereof, e.g., a fragment without the signal sequence or prodomain.

The mRNA sequences of human and murine MMP-9 may be found at GenBank Accession Nos NM_{—}004994 and NM_{—}013599, respectively. The sequences of human and mouse MMP-9 mRNAs are as follows:

| SEQ ID NO: 11: human MMP-9 mRNA | |
|---|---|
| 1 | agacacctct gccctcacca tgagcctctg gcagcccctg gtcctggtgc tcctggtgct |
| 61 | gggctgctgc tttgctgccc ccagacagcg ccagtccacc cttgtgctct tccctggaga |
| 121 | cctgagaacc aatctcaccg acaggcagct ggcagaggaa tacctgtacc gctatggtta |
| 181 | cactcgggtg gcagagatgc gtggagagtc gaaatctctg gggcctgcgc tgctgcttct |
| 241 | ccagaagcaa ctgtccctgc ccgagaccgg tgagctggat agcgccacgc tgaaggccat |
| 301 | gcgaacccca cggtgcgggg tcccagacct gggcagattc caaacctttg agggcgacct |
| 361 | caagtggcac caccacaaca tcacctattg gatccaaaac tactcggaag acttgccgcg |
| 421 | ggcggtgatt gacgacgcct ttgcccgcgc cttcgcactg tggagcgcgg tgacgccgct |
| 481 | caccttcact cgcgtgtaca gccgggacgc agacatcgtc atccagtttg gtgtcgcgga |
| 541 | gcacggagac gggtatccct tcgacgggaa ggacgggctc ctggcacacg cctttcctcc |
| 601 | tggccccggc attcagggag acgcccattt cgacgatgac gagttgtggt ccctgggcaa |
| 661 | gggcgtcgtg gttccaactc ggtttggaaa cgcagatggc gcggcctgcc acttcccctt |
| 721 | catcttcgag ggccgctcct actctgcctg caccaccgac ggtcgctccg acggcttgcc |
| 781 | ctggtgcagt accacggcca actacgacac cgacgaccgg tttggcttct gccccagcga |
| 841 | gagactctac acccaggacg gcaatgctga tgggaaaccc tgccagtttc cattcatctt |
| 901 | ccaaggccaa tcctactccg cctgcaccac ggacggtcgc tccgacggct accgctggtg |
| 961 | cgccaccacc gccaactacg accgggacaa gctcttcggc ttctgcccga cccgagctga |
| 1021 | ctcgacggtg atggggggca actcggcggg ggagctgtgc gtcttcccct tcactttcct |
| 1081 | gggtaaggag tactcgacct gtaccagcga gggccgcgga gatgggcgcc tctggtgcgc |
| 1141 | taccacctcg aactttgaca gcgacaagaa gtggggcttc tgcccggacc aaggatacag |
| 1201 | tttgttcctc gtggcggcgc atgagttcgg ccacgcgctg ggcttagatc attcctcagt |
| 1261 | gccggaggcg ctcatgtacc ctatgtaccg cttcactgag gggcccccct tgcataagga |
| 1321 | cgacgtgaat ggcatccggc acctctatgg tcctcgccct gaacctgagc cacggcctcc |
| 1381 | aaccaccacc acaccgcagc ccacggctcc cccgacggtc tgccccaccg gaccccccac |
| 1441 | tgtccacccc tcagagcgcc ccacagctgg ccccacaggt cccccctcag ctggccccac |
| 1501 | aggtcccccc actgctggcc cttctacggc cactactgtg cctttgagtc cggtggacga |
| 1561 | tgcctgcaac gtgaacatct tcgacgccat cgcggagatt gggaaccagc tgtatttgtt |
| 1621 | caaggatggg aagtactggc gattctctga gggcaggggg agccggccgc agggcccctt |
| 1681 | ccttatcgcc gacaagtggc ccgcgctgcc ccgcaagctg gactcggtct ttgaggagcg |
| 1741 | gctctccaag aagcttttct tcttctctgg gcgccaggtg tgggtgtaca caggcgcgtc |
| 1801 | ggtgctgggc ccgaggcgtc tggacaagct gggcctggga gccgacgtgg cccaggtgac |
| 1861 | cggggccctc cggagtggca gggggaagat gctgctgttc agcgggcggc gcctctggag |
| 1921 | gttcgacgtg aaggcgcaga tggtggatcc ccggagcgcc agcgaggtgg accggatgtt |
| 1981 | ccccggggtg cctttggaca cgcacgacgt cttccagtac cgagagaaag cctatttctg |
| 2041 | ccaggaccgc ttctactggc gcgtgagttc ccggagtgag ttgaaccagg tggaccaagt |
| 2101 | gggctacgtg acctatgaca tcctgcagtg ccctgaggac tagggctccc gtcctgcttt |
| 2161 | ggcagtgcca tgtaaatccc cactgggacc aaccctgggg aaggagccag tttgccggat |
| 2221 | acaaactggt attctgttct ggaggaaagg gaggagtgga ggtgggctgg gccctctctt |
| 2281 | ctcacctttg ttttttgttg gagtgtttcta ataaacttg gattctctaa cctttaaaaa |
| 2341 | aaaaaaaaaa aaaaaaaaaa aaaaaaaaaaa aaaaaaaaa aaaaaaa |
| SEQ ID NO: 12: mouse MMP-9 mRNA | |
| 1 | ctcaccatga gtccctggca gcccctgctc ctggctctcc tggctttcgg ctgcagctct |
| 61 | gctgcccctt accagcgcca gccgactttt gtggtcttcc ccaaagacct gaaaacctcc |
| 121 | aacctcacgg acacccagct ggcagaggca tacttgtacc gctatggtta cacccgggcc |
| 181 | gcccagatga tgggagagaa gcagtctcta cggccggctt tgctgatgct tcagaagcag |
| 241 | ctctccctgc cccagactgg tgagctggac agccagacac taaaggccat tcgaacacca |
| 301 | cgctgtggtg tcccagacgt gggtcgattc caaaccttca aaggcctcaa gtgggaccat |
| 361 | cataacatca catactggat ccaaaactac tctgaagact tgccgcgaga catgatcgat |
| 421 | gacgccttcg cgcgcgcctt cgcggtgtgg ggcgaggtgg cacccctcac cttcacccgc |
| 481 | gtgtacggac ccgaagcgga cattgtcatc cagtttggtg tcgcggagca cggagacggg |
| 541 | tatcccttcg acggcaagga cggccttctg gcacacgcct ttccccctgg cgccggcgtt |
| 601 | cagggagatg cccatttcga cgacgacgag ttgtggtcgc tgggcaaagg cgtcgtgatc |
| 661 | cccacttact atggaaactc aaatggtgcc ccatgtcact ttcccttcac cttcgaggga |
| 721 | cgctcctatt cggcctgcac cacagacggc cgcaacgacg gcacgccttg gtgtagcaca |
| 781 | acagctgact acgataagga cggcaaattt ggtttctgcc ctagtgagag actctacacg |
| 841 | gagcacggca acggagaagg caaaccctgt gtgttcccgt tcatctttga gggccgctcc |
| 901 | tactctgcct gcaccactaa aggccgctcg gatggttacc gctggtgcgc caccacagcc |
| 961 | aactatgacc aggataaact gtatggcttc tgccctaccc gagtggacgc gaccgtagtt |
| 1021 | gggggcaact cggcaggaga gctgtgcgtc ttccccttcg tcttcctggg caagcagtac |
| 1081 | tcttcctgta ccagcgacgg ccgcagggat gggcgcctct ggtgtgcgac cacatcgaac |
| 1141 | ttcgacactg acaagaagtg gggtttctgt ccagaccaag ggtacagcct gttcctggtg |
| 1201 | gcagcgcacg agttcggcca tgcactgggc ttagatcatt ccagcgtgcc ggaagcgctc |
| 1261 | atgtacccgc tgtatagcta cctcgagggc ttccctctga ataaagacga catagacggc |
| 1321 | atccagtatc tgtatggtcg tggctctaag cctgacccaa ggcctccagc caccaccaca |
| 1381 | actgaaccac agccgacagc acctcccact atgtgtccca ctatacctcc cacggcctat |
| 1441 | cccacagtgg gccccacggt tggccctaca ggcgccccct cacctggccc cacaagcagc |
| 1501 | ccgtcacctg gccctacagg cgccccctca cctggcccta cagcgccccc tactgcgggc |
| 1561 | tcttctgagg cctctacaga gtctttgagt ccggcagaca atccttgcaa tgtggatgtt |
| 1621 | tttgatgcta ttgctgagat ccagggcgct ctgcatttct tcaaggacgg ttggtactgg |
| 1681 | aagttcctga atcatagagg aagcccatta cagggcccct tccttactgc ccgcacgtgg |
| 1741 | ccagccctgc ctgcaacgct ggactccgcc tttgaggatc cgcagaccaa gagggttttc |
| 1801 | ttcttctctg gacgtcaaat gtgggtgtac acaggcaaga ccgtgctggg ccccaggagt |
| 1861 | ctggataagt tgggtctagg cccagaggta acccacgtca gcgggcttct cccgcgtcgt |
| 1921 | ctcgggaagg ctctgctgtt cagcaagggg cgtgtctgga gattcgactt gaagtctcag |
| 1981 | aaggtggatc cccagagcgt cattcgcgtg gataaggagt tctctggtgt gccctggaac |
| 2041 | tcacacgaca tcttccagta ccaagacaaa gcctatttct gccatggcaa attcttctgg |
| 2101 | cgtgtgagtt tccaaaatga ggtgaacaag gtggaccatg aggtgaacca ggtggacgac |
| 2161 | gtgggctacg tgacctacga cctcctgcag tgcccttgaa ctagggctcc ttctttgctt |
| 2221 | caaccgtgca gtgcaagtct ctagagacca ccaccaccac caccacacac aaaccccatc |
| 2281 | cgagggaaag gtgctagctg gccaggtaca gactggtgat ctcttctaga gactgggaag |
| 2341 | gagtggaggc aggcagggct ctctctgccc accgtccttt cttgttggac tgtttctaat |
| 2401 | aaacacggat ccccaacctt ttccagctac tttagtcaat cagcttatct gtagttgcag |
| 2461 | atgcatccga gcaagaagac aactttgtag ggtggattct gaccttttat ttttgtgtgg |
| 2521 | cgtctgagaa ttgaatcagc tggcttttgt gacaggcact tcaccggcta aaccacctct |
| 2581 | cccgactcca gcccttttat ttattatgta tgaggttatg ttcacatgca tgtatttaac |
| 2641 | ccacagaatg cttactgtgt gtcgggcgcg gctccaaccg ctgcataaat attaaggtat |
| 2701 | tcagttgccc ctactggaag gtattatgta actatttctc tcttacattg gagaacacca |
| 2761 | ccgagctatc cactcatcaa acatttattg agagcatccc tagggagcca ggctctctac |
| 2821 | tgggcgttag ggacagaaat gttggttctt ccttcaagga ttgctcagag attctccgtg |
| 2881 | tcctgtaaat ctgctgaaac cagaccccag actcctctct ctcccgagag tccaactcac |
| 2941 | tcactgtggt tgctggcagc tgcagcatgc gtatacagca tgtgtgctag agaggtagag |
| 3001 | ggggtctgtg cgttatggtt caggtcagac tgtgtcctcc aggtgagatg acccctcagc |
| 3061 | tggaactgat ccaggaagga taaccaagtg tcttcctggc agtctttttt aaataaatga |
| 3121 | ataaatgaat atttacttaa aaaaaaaaaa aaaaaaaaaa aaaaaaaaaa aaaaaaaaaa |
| 3181 | aaaaa |

An exemplary MMP-9 gene can consist of or comprise the human or mouse MMP-9 mRNA sequence, a sequence that is 80%, 85%, 90%, 95%, 96%, 97%, 98%, or 99% identical to one of these sequences, or a fragment thereof.

Methods of evaluating levels of gene expression and protein activity, as well as evaluating the amounts of gene or protein molecules in a sample, are well-known in the art. Exemplary methods by which the expression of the MMP-14, MMP-2, TIMP (e.g., TIMP-1) or MMP-9 genes or the activity of the MMP-14, MMP-2, TIMP (e.g., TIMP-1) or MMP-9 proteins may be determined are further described below.

In certain embodiments, a method of evaluating the expression and/or activity of MMP-14, MMP-2, TIMP (e.g., TIMP-1), and/or MMP-9 in a cell may comprise a) determining in the cell the level of expression and/or activity of MMP-14, MMP-2, TIMP (e.g., TIMP-1), and/or MMP-9. The method may in certain embodiments further comprise calculating a ratio of the expression and/or activity level of two or more of MMP-14, MMP-2, TIMP (e.g., TIMP-1), and MMP-9, for example, MMP-9 or MMP-2 expression in relation to TIMP (e.g., TIMP-1) expression from the determined levels. In some embodiments, the ratio of MMP-9/TIMP (e.g., TIMP-1) is determined, wherein a ratio higher than 1 (e.g., +1.5, +2, +2.5, +3 etc.) indicates a subject may have a poor response to MMP-14 inhibition and a ratio ≦1 indicates a subject is a good candidate for treatment with an MMP-14 inhibitor. In other embodiments, the ratio of MMP-2/TIMP (e.g., TIMP-1) is determined, wherein a ratio higher than 1 (e.g., +1.5, +2, +2.5, +3 etc.) indicates a subject is a good candidate for treatment, while a ratio ≦1 indicates a subject may have a poor response to an MMP-14 inhibitor. In another embodiment, a subject having high expression levels of MMP-2 is determined to be a good candidate for treatment with an MMP-14 inhibitor, while a subject having low expression levels of MMP-2 is expected to have a poor response to MMP-14 inhibitory strategies.

The above-described method may further comprise b) comparing the determined level of expression and/or activity of MMP-14, MMP-2, TIMP (e.g., TIMP-1), MMP-9, or the ratio of the level of expression and/or activity of two of, MMP-14, MMP-2, TIMP (e.g., TIMP-1), and MMP-9, e.g., the ratio of MMP-9 or MMP-2 expression to TIMP (e.g., TIMP-1) expression with at least one reference set of levels of expression and/or activity of, or ratio of, MMP-14, MMP-2, TIMP (e.g., TIMP-1), and MMP-9, wherein the reference set indicates the state of the cell associated with the particular level of expression and/or activity of, or ratio of two of MMP-14, MMP-2, TIMP (e.g., TIMP-1) and MMP-9, e.g., the ratio of the level of MMP-9 or MMP-2 expression to TIMP (e.g., TIMP-1) expression.

Comparison to a reference set or profile is particularly useful in applications of the above-described methods, for example, when they are used in methods for diagnosing and prognosing cancer in a subject, or for screening candidate therapeutics for their efficacy in treating cancer or for stratifying patients based on their risk for or stage of cancer or for selecting a therapy for a patient having or suspected of having cancer. In certain preferred embodiments, the cancer is a cancer described herein, e.g., a cancer selected from the group consisting of: osteotropic cancer, melanoma, pancreatic cancer, breast cancer, lung cancer, colon cancer, gastric cancer, and prostate cancer.

Comparison of the expression and/or activity level of MMP-14, MMP-2, TIMP (e.g., TIMP-1), and/or MMP-9, or ratio of the level of expression and/or activity of two of, MMP-14, MMP-2, TIMP (e.g., TIMP-1) and MMP-9, e.g., the ratio of level of MMP-9 or MMP-2 expression to TIMP (e.g., TIMP-1) expression, with reference expression and/or activity levels, or ratios, e.g., expression and/or activity levels in diseased cells of a subject having cancer or in normal counterpart cells, is preferably conducted using computer systems. In one embodiment, expression and/or activity levels are obtained in two cells and these two sets of expression and/or activity levels are introduced into a computer system for comparison. In a preferred embodiment, one set of expression and/or activity levels is entered into a computer system for comparison with values that are already present in the computer system, or in computer-readable form that is then entered into the computer system.

In one embodiment, the invention provides computer readable forms of the gene expression or protein activity profile data of the invention, or of values corresponding to the level of expression and/or activity of, or ratios of the level of expression and/or activity of, MMP-14, MMP-2, TIMP (e.g., TIMP-1) and/or MMP-9. In other embodiments, the invention provides computer readable forms of the gene expression or protein activity profile data of the invention, or of values corresponding to the ratios of the level of expression and/or activity of, MMP-9/TIMP or MMP-2/TIMP (e.g., TIMP-1). The values may be, for example, mRNA expression levels or AQUA™ scores. The values may also be mRNA levels, AQUA™ scores, or other measure of gene expression and/or protein activity normalized relative to a reference gene whose expression or protein whose activity is constant in numerous cells under numerous conditions. In other embodiments, the values in the computer are ratios of, or differences between, normalized or non-normalized levels in different samples.

The profile data may be in the form of a table, such as an Excel table. The data may be alone, or it may be part of a larger database, e.g., comprising other profiles. For example, the profile data of the invention may be part of a public database. The computer readable form may be in a computer. In another embodiment, the invention provides a computer displaying the profile data.

In one embodiment, the invention provides methods for determining the similarity between the level of expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) in a first cell, e.g., a cell of a subject, and that in a second cell, comprising obtaining the level of expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) in a first cell and entering these values into a computer comprising a database including records comprising values corresponding to levels of expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression), in a second cell, and processor instructions, e.g., a user interface, capable of receiving a selection of one or more values for comparison purposes with data that is stored in the computer. The computer may further comprise a means for converting the comparison data into a diagram or chart or other type of output.

In another embodiment, at least one value representing the expression and/or activity level of MMP-14, MMP-2, TIMP (e.g., TIMP-1) and/or MMP-9, or ratio of the level of expression and/or activity of two of MMP-14, MMP-2, TIMP (e.g., TIMP-1) and/or MMP-9 (e.g., the ratio of the level of MMP-9 or MMP-2 expression to TIMP (e.g., TIMP-1) expression) is entered into a computer system, comprising one or more databases with reference expression and/or activity levels, or ratios, obtained from more than one cell. For example, a computer may comprise expression and/or activity and/or ratio data of diseased and normal cells. Exemplary ratio data includes e.g., MMP-9/TIMP (e.g., TIMP-1) ratios or MMP-2/TIMP (e.g., TIMP-1) ratios. Instructions are provided to the computer, and the computer is capable of comparing the data entered with the data in the computer to determine whether the data entered is more similar to that of a normal cell or of a diseased cell.

In another embodiment, the computer comprises values of expression and/or activity levels, or ratios, in cells of subjects at different stages of cancer and the computer is capable of comparing expression and/or activity and/or ratio data entered into the computer with the data stored, and produce results indicating to which of the expression and/or activity and/or ratio profiles in the computer, the one entered is most similar, such as to determine the stage of cancer in the subject.

In yet another embodiment, the reference expression and/or activity and/or ratio profiles in the computer are expression and/or activity and/or ratio profiles from cells of one or more subjects having cancer, which cells are treated in vivo or in vitro with a drug used for therapy of cancer. Upon entering of expression and/or activity and/or ratio data of a cell of a subject treated in vitro or in vivo with the drug, the computer is instructed to compare the data entered to the data in the computer, and to provide results indicating whether the expression and/or activity data input into the computer are more similar to those of a cell of a subject that is responsive to the drug or more similar to those of a cell of a subject that is not responsive to the drug. Thus, the results indicate whether the subject is likely to respond to the treatment with the drug (e.g., more likely to respond than not, e.g., greater than 50% likelihood of responding) or unlikely to respond to it (e.g., greater than 50% likelihood of not responding).

In one embodiment, the invention provides systems comprising a means for receiving expression and/or activity and/or ratio data for one or a plurality of genes and/or protein; a means for comparing the expression and/or activity and/or ratio data from each of said one or plurality of genes and/or proteins to a common reference frame; and a means for presenting the results of the comparison. A system may further comprise a means for clustering the data.

In another embodiment, the invention provides computer programs for analyzing expression and/or activity and/or ratio data comprising (a) a computer code that receives as input expression and/or activity and/or ratio data for at least one gene and (b) a computer code that compares said expression and/or activity and/or ratio data from each gene to a common reference frame.

The invention also provides machine-readable or computer-readable media including program instructions for performing the following steps: (a) comparing at least one value corresponding to the expression and/or activity level of, or ratio of the level of expression and/or activity of two of, MMP-14, MMP-2, TIMP (e.g., TIMP-1), and/or MMP-9 in a query cell with a database including records comprising reference expression and/or activity and/or ratio data of one or more reference cells and an annotation of the type of cell; and (b) indicating to which cell the query cell is most similar based on similarities of expression and/or activity profiles and/or ratios. The reference cells may be, e.g., cells from subjects at different stages of cancer. The reference cells may also be, e.g., cells from subjects responding or not responding to a particular drug treatment and optionally incubated in vitro or in vivo with the drug.

The reference cells may also be cells from subjects responding or not responding to several different treatments, and the computer system indicates a preferred treatment for the subject. Accordingly, the invention provides methods for selecting a therapy for a patient having cancer; the methods comprising: (a) providing the level of expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) in a diseased cell of the patient; (b) providing a plurality of reference profiles, each associated with a therapy; and (c) selecting the reference profile most similar to the subject expression and/or activity profile, or ratio, to thereby select a therapy for said patient. In a preferred embodiment step (c) is performed by a computer. The most similar reference profile or ratio may be selected by weighing a comparison value of the plurality using a weight value associated with the corresponding expression and/or activity data, or ratio. In certain embodiments, the reference profile is selected by comparing the expressional ratio of MMP-9/TIMP (e.g., TIMP-1) or MMP-2/TIMP (e.g., TIMP-1).

A computer readable medium may further comprise a pointer to a descriptor of a stage of cancer or to a treatment for cancer.

In operation, the means for receiving expression and/or activity data, or ratios, the means for comparing the expression and/or activity data, or ratios, the means for presenting, the means for normalizing, and the means for clustering within the context of the systems of the present invention may involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that may cause a computer to function in the particular fashion described herein.

Those skilled in the art will understand that the systems and methods of the present invention may be applied to a variety of systems, including IBM®-compatible personal computers running MS-DOS® or Microsoft WINDOWS®. In an exemplary implementation, expression profiles are compared using a method described in U.S. Pat. No. 6,203,987. A user first loads expression profile or ratio data into the computer system. Geneset profile or ratio definitions are loaded into the memory from the storage media or from a remote computer, preferably from a dynamic geneset database system, through the network. Next the user causes execution of projection software which performs the steps of converting expression and/or activity profile, or ratio, to projected expression and/or activity profiles or ratios. The projected expression and/or activity profiles, or ratios, are then displayed.

In yet another exemplary implementation, a user first leads a projected profile or ratio into the memory. The user then causes the loading of a reference profile or ratio into the memory. Next, the user causes the execution of comparison software which performs the steps of objectively comparing the profiles or ratios.

Exemplary diagnostic tools and assays are set forth below, which comprise the above-described methodology.

In one embodiment, the invention provides methods for determining whether a subject has or is likely to develop cancer, comprising determining the level of expression and/or activity of MMP-14, MMP-2, TIMP (e.g., TIMP-1), and/or MMP-9 in a cell of the subject and comparing these levels of expression and/or activity, or ratio of the levels, with the levels of expression of or ratios of MMP-14, MMP-2, TIMP (e.g., TIMP-1), and/or MMP-9 in a diseased cell of a subject known to have cancer, such that a similar level of expression and/or activity of, or ratio of, MMP-14, MMP-2, TIMP (e.g., TIMP-1), and/or MMP-9 is indicative that the subject has or is likely to develop cancer or at least a symptom thereof. In a preferred embodiment, the cell is essentially of the same type as that which is diseased in the subject.

In another embodiment the expression and/or activity profiles, or ratios, of genes in the cell may be used to confirm that a subject has a specific type of cancer, and in particular, that the subject does not have a related disease or disease with similar symptoms. This may be important, in particular, in designing an optimal therapeutic regimen for the subject. It has been described in the art that expression and/or activity profiles or ratios may be used to distinguish one type of disease from a similar disease. For example, two subtypes of non-Hodgkin's lymphomas, one of which responds to current therapeutic methods and the other one which does not, could be differentiated by investigating 17,856 genes in specimens of patients suffering from diffuse large B-cell lymphoma (Alizadeh et al. Nature (2000) 405:503). Similarly, subtypes of cutaneous melanoma were predicted based on profiling 8150 genes (Bittner et al. Nature (2000) 406:536). In this case, features of the highly aggressive metastatic melanomas could be recognized. Numerous other studies comparing expression and/or activity profiles or ratios of cancer cells and normal cells have been described, including studies describing expression profiles distinguishing between highly and less metastatic cancers and studies describing new subtypes of diseases, e.g., new tumor types (see, e.g., Perou et al. (1999) PNAS 96: 9212; Perou et al. (2000) Nature 606:747; Clark et al. (2000) Nature 406:532; Alon et al. (1999) PNAS 96:6745; Golub et al. (1999) Science 286:531). Such distinction is known in the art as “differential diagnosis”.

In yet another embodiment, the invention provides methods for determining the stage of cancer, i.e., for “staging” cancer. It is thought that the level of expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) changes with the stage of the disease. This could be confirmed, e.g., by analyzing the level of expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) in subjects having cancer at different stages, as determined by traditional methods. For example, the expression profile of a diseased cell in subjects at different stages of the disease may be determined as described herein. Then, to determine the stage of cancer in a subject, the level of expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression), which varies with the stage of the disease, is determined. A similar level of expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) between that in a subject and that in a reference profile of a particular stage of the disease, indicates that the disease of the subject is at the particular stage.

Similarly, the methods may be used to determine the stage of the disease in a subject undergoing therapy, and thereby determine whether the therapy is effective. Accordingly, in one embodiment, the level of expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) is determined in a subject before the treatment and several times during the treatment. For example, a sample of RNA may be obtained from the subject and analyzed before the beginning of the therapy and every 12, 24, 36, 48, 60, or 72 hours during the therapy. Alternatively or in addition, samples may be analyzed once a week or once a month or once a year, e.g., over the course of the therapy. Changes in expression and/or activity levels of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) over time and relative to diseased cells and normal cells will indicate whether the therapy is effective.

Further, the methods may be used to determine the stage of the disease in a subject after undergoing therapy, e.g., and thereby determine whether the therapy was effective and/or whether the disease is re-developing (e.g., whether the disease has returned, e.g., whether the disease has relapsed). Accordingly, in one embodiment, the level of expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) is determined in a subject during and/or immediately after the treatment and/or several times after the treatment. For example, a sample of RNA may be obtained from the subject and analyzed at the end of the therapy and once a week, once a month or once a year, e.g., for the next 1, 2, 3, 4, or 5 years. Changes in expression and/or activity levels of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) over time and relative to diseased cells and normal cells can indicate whether the therapy was effective, and/or whether the disease is re-developing.

In yet another embodiment, the invention provides methods for determining the likelihood of success of a particular therapy in a subject having cancer. In one embodiment, a subject is started on a particular therapy, and the effectiveness of the therapy is determined, e.g., by determining the level of expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) in a cell of the subject. A normalization of the level of expression and/or activity of MMP-14, MMP-2, TIMP (e.g., TIMP-1) and/or MMP-9, or the ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression), i.e., a change in the expression and/or activity of level, or ratio, of the gene(s) such that their level of expression and/or activity or ratio, resembles more that of a non diseased cell, indicates that the treatment should be effective in the subject. In certain embodiments, the invention provides methods for determining whether a subject has a cancer that is likely to respond to treatment with a MMP-14 inhibitor, comprising determining the ratio of the level of expression of MMP-9/TIMP and/or MMP-2/TIMP in a cell of the subject and comparing the ratio to those ratio in a diseased cell of a subject known to have cancer. Typically, expressional ratios for MMP-9/TIMP less than or equal to 1 and/or expressional ratios of MMP-2/TIMP greater than 1 indicate that the subject is likely to respond to MMP-14 inhibition.

Prediction of the outcome of a treatment in a subject may also be undertaken in vitro. In one embodiment, cells are obtained from a subject to be evaluated for responsiveness to the treatment, and incubated in vitro with the therapeutic drug. The level of expression and/or activity of MMP-14, MMP-2, TIMP (e.g., TIMP-1), and/or MMP-9 is then measured in the cells and these values are compared to the level of expression and/or activity of MMP-14, MMP-2, TIMP (e.g., TIMP-1), and/or MMP-9 in a cell which is the normal counterpart cell of a diseased cell. The level of expression and/or activity may also be compared to that in a normal cell. In certain embodiments, the ratio of the level of expression and/or activity of two of MMP-14, MMP-2, TIMP (e.g., TIMP-1), and/or MMP-9 may be used. The comparative analysis is preferably conducted using a computer comprising a database of expression and/or activity profiles of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) in the cells of the subject after incubation with the drug that is similar to their level of expression and/or activity, or ratio of the level of expression and/or activity, in a normal cell and different from that in a diseased cell is indicative that it is likely that the subject will respond positively to a treatment with the drug. On the contrary, a level of expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) in the cells of the subject after incubation with the drug that is similar to their level of expression and/or activity, or ratio, in a diseased cell and different from that in a normal cell is indicative that it is likely that the subject will not respond positively to a treatment with the drug, e.g., an MMP-14 inhibitor.

Since it is possible that a drug does not act directly on the diseased cells, but is, e.g., metabolized, or acts on another cell which then secretes a factor that will effect the diseased cells, the above assay may also be conducted in a tissue sample of a subject, which contains cells other than the diseased cells. For example, a tissue sample comprising diseased cells is obtained from a subject; the tissue sample is incubated with the potential drug; optionally one or more diseased cells are isolated from the tissue sample, e.g., by microdissection or Laser Capture Microdissection (LCM, see infra); and the expression level of MMP-14, MMP-2, TIMP (e.g., TIMP-1), and/or MMP-9 is examined.

Provided also are methods for selecting a therapy for cancer for a patient from a selection of several different treatments. Certain subjects having cancer may respond better to one type of therapy than another type of therapy. In a preferred embodiment, the method comprises comparing the expression and/or activity level of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) in the patient with that in cells of subjects treated in vitro or in vivo with one of several therapeutic drugs, which subjects are responders or non responders to one of the therapeutic drugs, and identifying the cell which has the most similar level of expression and/or activity of, or ratio of the level of expression and/or activity of MMP-14, MMP-2, TIMP (e.g., TIMP-1), and/or MMP-9 to that of the patient, to thereby identify a therapy for the patient. The method may further comprise administering the therapy identified to the subject.

In some embodiments, the method includes selecting a patient for treatment with a therapeutic drug that has an expression and/or activity level of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) similar to a responder, and administering the therapeutic drug to the patient. In some embodiments, the method includes selecting a patient for treatment with a first therapeutic drug when the patient has an expression and/or activity level of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) similar to a non responder to a second therapeutic drug, and administering the first therapeutic drug to the patient.

Methods of Evaluating the Expression and/or Activity of MMP-14, MMP-2, TIMP (e.g., TIMP-1) and/or MMP-9

The methods of diagnosing and prognosing cancer by evaluating the level of expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression) and methods of screening candidate therapeutic agents which modulate the expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression), described above, comprise determining the level of expression and/or activity of MMP-14, MMP-2, TIMP (e.g., TIMP-1) and/or MMP-9, or ratio of the level of expression and/or activity of two of, MMP-14, MMP-2, TIMP (e.g., TIMP-1) and/or MMP-9 (e.g., the ratio of the level of MMP-9 or MMP-2 expression to TIMP (e.g., TIMP-1) expression). In some embodiments, the level of expression or activity of MMP-14, MMP-9 and TIMP-1 are determined. In some embodiments, the level of expression or activity of MMP-14 and the ratio of expression or activity of MMP-9 to TIMP (e.g., TIMP-1) are determined. In some embodiments, the level or activity of MMP-2 is determined and/or the presence or absence of a mutation, e.g., a germline mutation, associated with increased MMP-2 levels, e.g., a germline mutation in the CDKN2A gene or a protein encoded by that gene.

Methods for determining the expression level and ultimately the activity of MMP-14, MMP-2, TIMP (e.g., TIMP-1), and/or MMP-9 are well known in the art (and the ratio of such levels may be determined from the determined levels). For example, the expression level of MMP-14, MMP-2, TIMP (e.g., TIMP-1), and/or MMP-9 can be determined by reverse transcription-polymerase chain reaction (RT-PCR); dotblot analysis; Northern blot analysis and in situ hybridization. Alternatively, the level of MMP-14, MMP-2, TIMP (e.g., TIMP-1), and/or MMP-9 can be analyzed using an appropriate antibody. In certain embodiments, the amounts of MMP-14, MMP-2, TIMP (e.g., TIMP-1), and/or MMP-9 is determined using antibodies against MMP-14, MMP-2, TIMP (e.g., TIMP-1), and/or MMP-9.

In certain embodiments, the level of expression of MMP-14, MMP-2, TIMP (e.g., TIMP-1), and/or MMP-9 is determined by determining its AQUA™ score, e.g., by using the AQUA™ automated pathology system. AQUA™ (for Automated Quantitative Analysis) is a method of analysis of absolute measurement of protein expression in situ. This method allows measurements of protein expression within sub-cellular compartments that results in a number directly proportional to the number of molecules expressed per unit area. For example, to measure nuclear estrogen receptor (ER), the tissue is “masked” using keratin in one channel to normalize the area of tumor and to remove the stromal and other non-tumor material from analysis. Then an image is taken using DAPI to define a nuclear compartment. The pixels within the mask and within the DAPI-defined compartment are defined as nuclear. The intensity of expression of ER is then measured using a third channel. The intensity of that subset of pixels divided by the number of pixels (to normalize the area from spot to spot) to give an AQUA™ score. This score is directly proportional to the number of molecules of ER per unit area of tumor, as assessed by a standard curve of cell lines with known levels of ER protein expression. This method, including details of out-of-focus light subtraction imaging methods, is described in detail in a Nature Medicine paper (Camp, R. L., Chung, G. G. & Rimm, D. L. Automated subcellular localization and quantification of protein expression in tissue microarrays. Nat Med 8, 1323-7 (2002)), as well as U.S. Ser. No. 10/062,308, filed Feb. 1, 2002, both of which reference are incorporated herein by their entireties.

In other embodiments, methods of detecting the level of expression of MMP-14, MMP-2, TIMP (e.g., TIMP-1), and/or MMP-9 may comprise the use of a microarray. Arrays are often divided into microarrays and macroarrays, where microarrays have a much higher density of individual probe species per area. Microarrays may have as many as 1000 or more different probes in a 1 cm² area. There is no concrete cut-off to demarcate the difference between micro- and macroarrays, and both types of arrays are contemplated for use with the invention.

Microarrays are known in the art and generally consist of a surface to which probes that correspond in sequence to gene products (e.g., cDNAs, mRNAs, oligonucleotides) are bound at known positions. In one embodiment, the microarray is an array (e.g., a matrix) in which each position represents a discrete binding site for a product encoded by a gene (e.g., a protein or RNA), and in which binding sites are present for products of most or almost all of the genes in the organism's genome. In certain embodiments, the binding site or site is a nucleic acid or nucleic acid analogue to which a particular cognate cDNA can specifically hybridize. The nucleic acid or analogue of the binding site may be, e.g., a synthetic oligomer, a full-length cDNA, a less-than full length cDNA, or a gene fragment.

Although in certain embodiments the microarray contains binding sites for products of all or almost all genes in the target organism's genome, such comprehensiveness is not necessarily required. Usually the microarray will have binding sites corresponding to at least 100, 500, 1000, 4000 genes or more. In certain embodiments, arrays will have anywhere from about 50, 60, 70, 80, 90, or even more than 95% of the genes of a particular organism represented. The microarray typically has binding sites for genes relevant to testing and confirming a biological network model of interest. Several exemplary human microarrays are publicly available.

The probes to be affixed to the arrays are typically polynucleotides. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, which result in amplification of unique fragments (e.g., fragments that do not share more than 10 bases of contiguous identical sequence with any other fragment on the microarray). Computer programs are useful in the design of primers with the required specificity and optimal amplification properties. See, e.g., Oligo p1 version 5.0 (National Biosciences). In an alternative embodiment, the binding (hybridization) sites are made from plasmid or phage clones of genes, cDNAs (e.g., expressed sequence tags), or inserts therefrom (Nguyen et al., 1995, Genomics 29:207-209).

A number of methods are known in the art for affixing the nucleic acids or analogues to a solid support that makes up the array (Schena et al., 1995, Science 270:467-470; DeRisi et al., 1996, Nature Genetics 14:457-460; Shalon et al., 1996, Genome Res. 6:639-645; and Schena et al., 1995, Proc. Natl. Acad. Sci. USA 93:10539-11286).

Another method for making microarrays is by making high-density oligonucleotide arrays (Fodor et al., 1991, Science 251:767-773; Pease et al., 1994, Proc. Natl. Acad. Sci. USA 91:5022-5026; Lockhart et al., 1996, Nature Biotech 14:1675; U.S. Pat. Nos. 5,578,832; 5,556,752; and 5,510,270; Blanchard et al., 1996, 11: 687-90).

Other methods for making microarrays, e.g., by masking (Maskos and Southern, 1992, Nuc. Acids Res. 20:1679-1684), may also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989), could be used, as will be recognized by those of skill in the art.

The nucleic acids to be contacted with the microarray may be prepared in a variety of ways, and may include nucleotides of the subject invention. Such nucleic acids are often labeled fluorescently. Nucleic acid hybridization and wash conditions are chosen so that the population of labeled nucleic acids will specifically hybridize to appropriate, complementary nucleic acids affixed to the matrix. Non-specific binding of the labeled nucleic acids to the array can be decreased by treating the array with a large quantity of non-specific DNA—a so-called “blocking” step.

When fluorescently labeled probes are used, the fluorescence emissions at each site of a transcript array may be detected by scanning confocal laser microscopy. When two fluorophores are used, a separate scan, using the appropriate excitation line, is carried out for each of the two fluorophores used. Fluorescent microarray scanners are commercially available from Affymetrix, Packard BioChip Technologies, BioRobotics and many other suppliers. Signals are recorded, quantitated and analyzed using a variety of computer software.

According to the method of the invention, the relative abundance of an mRNA in two cells or cell lines is scored as a perturbation and its magnitude determined (i.e., the abundance is different in the two sources of mRNA tested), or as not perturbed (i.e., the relative abundance is the same). As used herein, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Present detection methods allow reliable detection of difference of an order of about 2-fold to about 5-fold, but more sensitive methods are expected to be developed.

In addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

In certain embodiments, the data obtained from such experiments reflects the relative expression of each gene represented in the microarray. Expression levels in different samples and conditions may now be compared using a variety of statistical methods.

In certain embodiments, the cell comprises a tissue sample, which may be present on a tissue microarray. For example, paraffin-embedded formalin-fixed specimens may be prepared, and punch “biopsy” cores taken from separate areas of the specimens. Each core may be arrayed into a separate recipient block, and sections cut and processed as previously described, for example, in Konenen, J. et al., Tissue microarrays for high-throughput molecular profiling of tumor specimens, (1987) Nat. Med. 4:844-7 and Chung, G. G. et al., Clin. Cancer Res. (In Press).

In other embodiments, the cell comprises a cell culture pellet, which may be present on a cell culture pellet microarray.

In certain embodiments, it is sufficient to determine the expression of one or only a few genes, as opposed to hundreds or thousands of genes. Although microarrays may be used in these embodiments, various other methods of detection of gene expression are available. This section describes a few exemplary methods for detecting and quantifying mRNA or polypeptide encoded thereby. Where the first step of the methods includes isolation of mRNA from cells, this step may be conducted as described above. Labeling of one or more nucleic acids may be performed as described above.

In one embodiment, mRNA obtained from a sample is reverse transcribed into a first cDNA strand and subjected to PCR, e.g., RT-PCR. House keeping genes, or other genes whose expression does not vary may be used as internal controls and controls across experiments. Following the PCR reaction, the amplified products may be separated by electrophoresis and detected. By using quantitative PCR, the level of amplified product will correlate with the level of RNA that was present in the sample. The amplified samples may also be separated on an agarose or polyacrylamide gel, transferred onto a filter, and the filter hybridized with a probe specific for the gene of interest. Numerous samples may be analyzed simultaneously by conducting parallel PCR amplification, e.g., by multiplex PCR.

“Dot blot” hybridization has gained wide-spread use, and many versions were developed (see, e.g., M. L. M. Anderson and B. D. Young, in Nucleic Acid Hybridization—A Practical Approach, B. D. Hames and S. J. Higgins, Eds., IRL Press, Washington D.C., Chapter 4, pp. 73-111, 1985).

In another embodiment, mRNA levels is determined by dot blot analysis and related methods (see, e.g., G. A. Beltz et al., in Methods in Enzymology, Vol. 100, Part B, R. Wu, L. Grossmam, K. Moldave, Eds., Academic Press, New York, Chapter 19, pp. 266-308, 1985). In one embodiment, a specified amount of RNA extracted from cells is blotted (i.e., non-covalently bound) onto a filter, and the filter is hybridized with a probe of the gene of interest. Numerous RNA samples may be analyzed simultaneously, since a blot may comprise multiple spots of RNA. Hybridization is detected using a method that depends on the type of label of the probe. In another dot blot method, one or more probes for a biomarker are attached to a membrane, and the membrane is incubated with labeled nucleic acids obtained from and optionally derived from RNA of a cell or tissue of a subject. Such a dot blot is essentially an array comprising fewer probes than a microarray.

Another format, the so-called “sandwich” hybridization, involves covalently attaching oligonucleotide probes to a solid support and using them to capture and detect multiple nucleic acid targets (see, e.g., M. Ranki et al. (1983) Gene, 21:77-85; A. M. Palva, et al, in UK Patent Application GB 2156074A, Oct. 2, 1985; T. M. Ranki and H. E. Soderlund in U.S. Pat. No. 4,563,419, Jan. 7, 1986; A. D. B. Malcolm and J. A. Langdale, in PCT WO 86/03782, Jul. 3, 1986; Y. Stabinsky, in U.S. Pat. No. 4,751,177, Jan. 14, 1988; T. H. Adams et al., in PCT WO 90/01564, Feb. 22, 1990; R. B. Wallace et al. (1979) Nucleic Acid Res. 6,11:3543; and B. J. Connor et al. (1983) PNAS 80:278-282). Multiplex versions of these formats are called “reverse dot blots.”

mRNA levels may also be determined by Northern blots. Specific amounts of RNA are separated by gel electrophoresis and transferred onto a filter which is then hybridized with a probe corresponding to the gene of interest. This method, although more burdensome when numerous samples and genes are to be analyzed, provides the advantage of being very accurate.

Another method for high throughput analysis of gene expression is the serial analysis of gene expression (SAGE) technique, first described in Velculescu et al. (1995) Science 270, 484-487. Among the advantages of SAGE is that it has the potential to provide detection of all genes expressed in a given cell type, provides quantitative information about the relative expression of such genes, permits ready comparison of gene expression of genes in two cells, and yields sequence information that may be used to identify the detected genes. Thus far, SAGE methodology has proved itself to reliably detect expression of regulated and nonregulated genes in a variety of cell types (Velculescu et al. (1997) Cell 88, 243-251; Zhang et al. (1997) Science 276, 1268-1272 and Velculescu et al. (1999) Nat. Genet. 23, 387-388.

Techniques for producing and probing nucleic acids are further described, for example, in Sambrook et al., Molecular Cloning: A Laboratory Manual (New York, Cold Spring Harbor Laboratory, 1989).

Alternatively, the level of expression of MMP-14, MMP-2, TIMP (e.g., TIMP-1), and/or MMP-9 is determined by in situ hybridization. In one embodiment, a tissue sample is obtained from a subject, the tissue sample is sliced, and in situ hybridization is performed according to methods known in the art, to determine the level of expression of MMP-14, MMP-2, TIMP (e.g., TIMP-1), and/or MMP-9.

In other methods, the level of expression of MMP-14, MMP-2, TIMP (e.g., TIMP-1), and/or MMP-9 is detected by measuring the level of protein encoded by the MMP-14, MMP-2, TIMP (e.g., TIMP-1), and/or MMP-9 gene. This may be done, e.g., by immunoprecipitation, ELISA, or immunohistochemistry using an agent, e.g., an antibody, that specifically detects the protein encoded by the gene. Other techniques include Western blot analysis. Immunoassays are commonly used to quantitate the levels of proteins in cell samples, and many other immunoassay techniques are known in the art. The invention is not limited to a particular assay procedure, and therefore is intended to include both homogeneous and heterogeneous procedures. Exemplary immunoassays which may be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, may be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

In the case of polypeptides which are secreted from cells, the level of expression of these polypeptides may be measured in biological fluids.

The above-described methods may be performed using cells grown in cell culture, or on cell or tissue specimens from a subject. Specimens may be obtained from an individual to be tested using either “invasive” or “non-invasive” sampling means. A sampling means is said to be “invasive” if it involves the collection of nucleic acids from within the skin or organs of an animal (including, especially, a murine, a human, an ovine, an equine, a bovine, a porcine, a canine, or a feline animal). Examples of invasive methods include blood collection, semen collection, needle biopsy, pleural aspiration, umbilical cord biopsy, etc. Examples of such methods are discussed by Kim, C. H. et al. (1992) J. Virol. 66:3879-3882; Biswas, B. et al. (1990) Annals NY Acad. Sci. 590:582-583; Biswas, B. et al. (1991) J. Clin. Microbiol. 29:2228-2233. It is also possible to obtain a cell sample from a subject, and then to enrich it in the desired cell type. For example, cells may be isolated from other cells using a variety of techniques, such as isolation with an antibody binding to an epitope on the cell surface of the desired cell type.

In certain embodiments, a single cell is used in the analysis. It is also possible to obtain cells from a subject and culture the cells in vitro, such as to obtain a larger population of cells from which RNA may be extracted. Methods for establishing cultures of non-transformed cells, i.e., primary cell cultures, are known in the art.

When analyzing from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA and proteins in the tissue and cells may quickly become degraded. Accordingly, in a preferred embodiment, the cells obtained from a subject are snap frozen as soon as possible.

Agents that Bind MMP-14, MMP-2, TIMP (e.g., TIMP-1), and/or MMP-9

Provided also are agents that bind MMP-14, MMP-2, TIMP (e.g., TIMP-1), and/or MMP-9 polypeptides. Preferably, such agents are anti-MMP-14, MMP-2 and/or MMP-9 antibodies or antigen-binding fragments thereof, including polyclonal and monoclonal antibodies, prepared according to conventional methodology. Antibodies and antigen-binding fragments thereof that bind MMP-14, MMP-2 and/or MMP-9 biomarkers are useful for determining MMP-14, MMP-2 and/or MMP-9 protein levels.

Antibodies and antigen-binding fragments thereof that bind MMP-14, MMP-2, TIMP (e.g., TIMP-1), and/or MMP-9 and are useful for determining MMP-14, MMP-2, TIMP (e.g., TIMP-1), and/or MMP-9 levels, include but are not limited to: antibodies or antigen-binding fragments thereof that bind specifically to a MMP-14, MMP-2, TIMP (e.g., TIMP-1) and/or MMP-9 or fragments or analogs thereof.

Significantly, as is well-known in the art, only a small portion of an antibody molecule, the paratrope, is involved in the binding of the antibody to its epitope (see, in general, Clark, W. R. (1986) The Experimental Foundations of Modern Immunology, Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, W. R. (1986) The Experimental Foundations of Modern Immunology, Wiley & Sons, Inc., New York; Roitt, I. (1991) Essential Immunology, 7th Ed., Blackwell Scientific Publications, Oxford). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Fully human monoclonal antibodies also can be prepared by immunizing mice transgenic for large portions of human immunoglobulin heavy and light chain loci. Following immunization of these mice (e.g., XENOMOUSE™ (Abgenix), HUMAB-MOUSE™ (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Thus, the invention involves polypeptides of numerous size and type that bind specifically to MMP-14, MMP-2, TIMP (e.g., TIMP-1) and/or MMP-9 polypeptides and nucleic acids. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to MMP-14, MMP-2, TIMP (e.g., TIMP-1), and/or MMP-9 molecules. This process can be repeated through several cycles of reselection of phage that bind to the MMP-14, MMP-2, TIMP (e.g., TIMP-1), and/or MMP-9 molecules. Repeated rounds lead to enrichment of phage bearing particular sequences. DNA sequence analysis can be conducted to identify the sequences of the expressed polypeptides. The minimal linear portion of the sequence that binds to the MMP-14, MMP-2, TIMP (e.g., TIMP-1), and/or MMP-9 molecules can be determined. One can repeat the procedure using a biased library containing inserts containing part of all of the minimal linear portion plus one or more additional degenerate residues upstream or downstream thereof. Yeast two-hybrid screening methods also may be used to identify polypeptides that bind to the MMP-14, MMP-2, TIMP (e.g., TIMP-1), and/or MMP-9 molecules. Thus, MMP-14, MMP-2, TIMP (e.g., TIMP-1), and/or MMP-9 molecules can be used to screen peptide libraries, including phage display libraries, to identify and select peptide binding partners of the MMP-14, MMP-2, TIMP (e.g., TIMP-1), and/or MMP-9 molecules.

Exemplary MMP-14 binding proteins that may be used either to detect MMP-14 or inhibit MMP-14 also include those M0031-C02, M0031-F01, M0033-H07, M0037-C09, M0037-D01, M0038-E06, M0038-F01, M0038-F08, M0039-H08, M0040-A06, M0040-A11, and M0043-G02. The amino acid sequences of exemplary Fab heavy chain (HC) and light chain (LC) variable regions of these binding proteins, and further descriptions of them and their discovery and production, are provided in pending application U.S. Ser. No. 11/648,423 (US 2007-0217997), which is hereby incorporated by reference herein in its entirety. Other exemplary MMP-14 binding proteins include DX-2400 and DX-2410. DX-2400 and M0038-F01 share HC and LC CDR amino acid sequences.

Exemplary MMP-9 binding proteins that may be used either to detect MMP-9 or inhibit MMP-9 include 539A-M0166-F10 and 539A-M0240-B03. The amino acid sequences of exemplary Fab heavy chain (HC) and light chain (LC) variable regions of these binding proteins, and further descriptions of them and their discovery and production, are provided in pending applications U.S. Ser. No. 61/033,075 and 61/054,938, which are hereby incorporated by reference herein in their entireties.

As detailed herein, the foregoing antibodies and other binding proteins may be used for example to isolate and identify MMP-14, MMP-2, TIMP (e.g., TIMP-1), and/or MMP-9 protein, e.g. to detect its expression in tissue samples. The antibodies may be coupled to specific diagnostic labeling agents for imaging of the protein or fragment thereof. Exemplary labels include, but are not limited to, labels which when fused to a MMP-14, MMP-2, TIMP (e.g., TIMP-1), and/or MMP-9 molecule produce a detectable fluorescent signal, including, for example, green fluorescent protein (GFP), enhanced green fluorescent protein (EGFP), Renilla reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED). In another embodiment, a cancer biomarker polypeptide is conjugated to a fluorescent or chromogenic label. A wide variety of fluorescent labels are available from and/or extensively described in the Handbook of Fluorescent Probes and Research Products 8^{th} Ed. (2001), available from Molecular Probes, Eugene, Oreg., as well as many other manufacturers.

In other embodiments, MMP-14, MMP-2, TIMP (e.g., TIMP-1) and/or MMP-9 is fused to a molecule that is readily detectable either by its presence or activity, including, but not limited to, luciferase, fluorescent protein (e.g., green fluorescent protein), chloramphenicol acetyl transferase, β-galactosidase, secreted placental alkaline phosphatase, β-lactamase, human growth hormone, and other secreted enzyme reporters.

Kits

The present invention provides kits for practice of the afore-described methods. In certain embodiments, kits may comprise antibodies against MMP-14, MMP-2, TIMP (e.g., TIMP-1), and/or MMP-9. In other embodiments, a kit may comprise appropriate reagents for determining the level of protein activity in the cells of a subject. In certain embodiments, the cell of a subject may be taken from a tumor biopsy.

In still other embodiments, a kit may comprise a microarray comprising probes of MMP-14, MMP-2, TIMP (e.g., TIMP-1), and/or MMP-9 genes or proteins. A kit may comprise one or more probes or primers for detecting the expression level of MMP-14, MMP-2, TIMP (e.g., TIMP-1) and/or MMP-9 and/or a solid support on which probes are attached and which may be used for detecting expression. A kit may further comprise controls, buffers, and instructions for use.

Kits may also comprise a library of MMP-14, MMP-2, TIMP (e.g., TIMP-1) and/or MMP-9 expression or activity levels associated with survival, response to therapy, stage of disease, etc., e.g., reference sets. In one embodiment, the kit comprises a computer readable medium on which is stored one or more measures of gene expression and/or protein activity associated with survival, response to therapy, stage of disease, etc., or at least values representing such measures of gene expression or protein activity associated with survival, response to therapy, stage of disease, etc. The kit may comprise ratio analysis software capable of being loaded into the memory of a computer system.

Kit components may be packaged for either manual or partially or wholly automated practice of the foregoing methods. In other embodiments involving kits, this invention contemplates a kit including compositions of the present invention, and optionally instructions for their use. Such kits may have a variety of uses, including, for example, imaging, diagnosis, therapy, and other applications.

### EXEMPLIFICATION

The present invention is further illustrated by the following examples which should not be construed as limiting in any way.

### Example 1

### Expression of MMPs in Various Cancer Cell Lines and Correlation to MMP-14 Inhibitor Efficacy

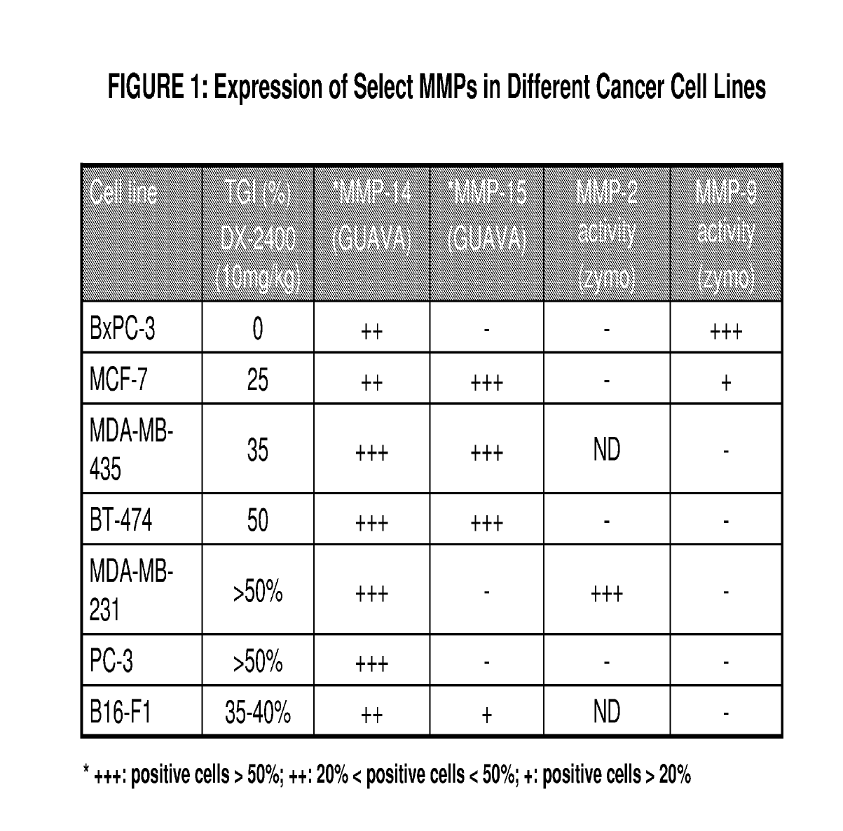

FIG. 1 illustrates the relative expression levels of various MMPs, including MMP-14 and MMP-2, in different cancer cell lines. MDA-MB-231 expresses both MMP-14 and MMP-2 in over 50% of cells. MDA-MB-435, BT-474 and PC-3 express only MMP-14 in over 50% of cells. BxPC-3 and B16-F1 express MMP-14 in between 20% and 50% of cells (but not MMP-2). The MCF-7 passage of cells used for these experiments express MMP-14 in between 20% and 50% of cells (but not MMP-2).

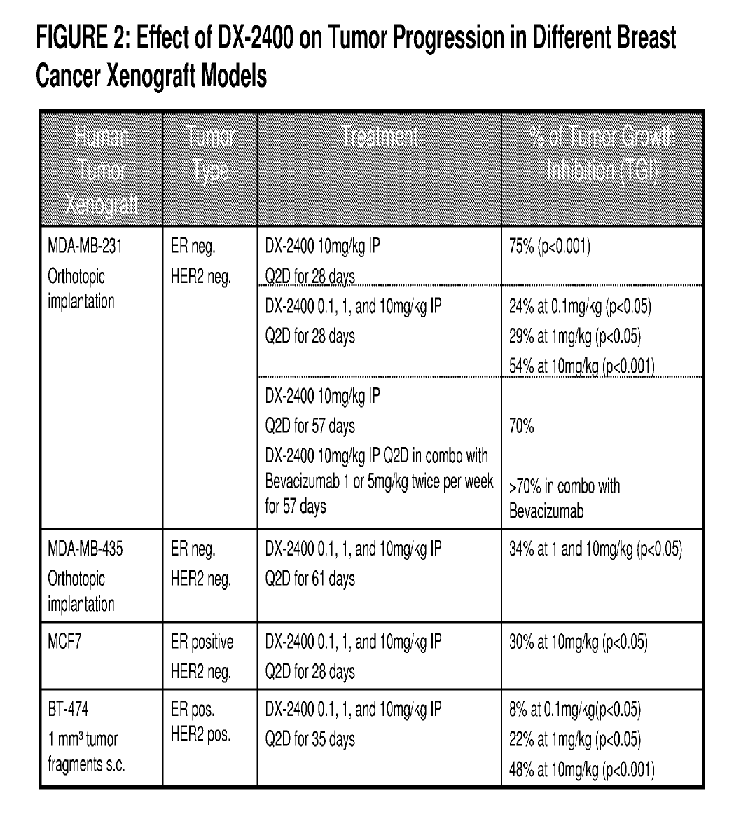

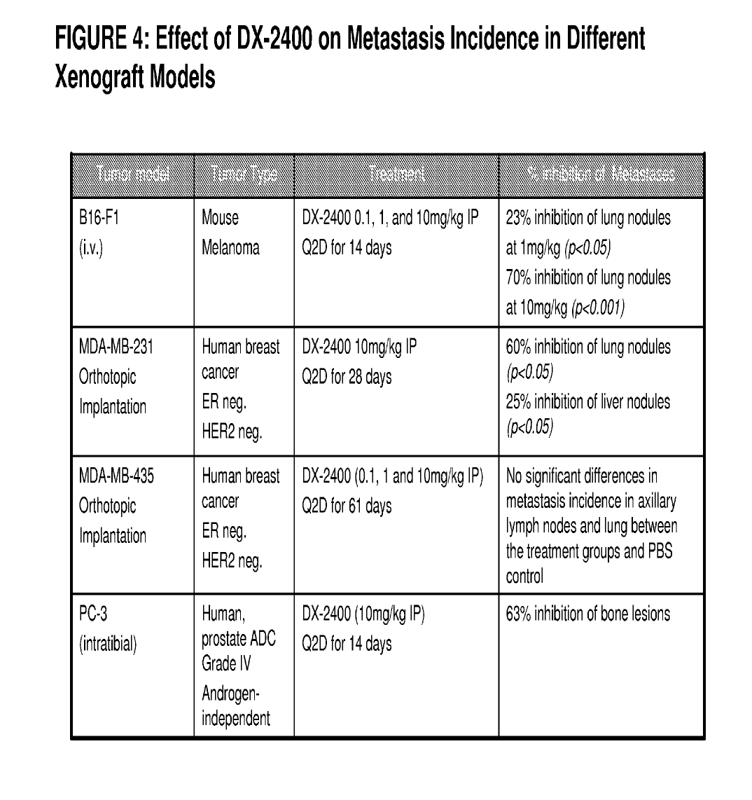

The effect of DX-2400, an MMP-14 inhibitor, in inhibiting tumor growth, was strongest in MDA-MB-231, MDA-MB-435, BT-474 and PC-3, all of which express MMP-14 in over 50% of cells (FIGS. 2 and 3). Further, DX-2400 had an effect on metastasis on certain cell lines expressing MMP-14 in at least 20% of cells (FIG. 4).

### Example 2

### Tumor Growth Data with MMP-14-Positive and MMP-14-Negative Cancer Cells

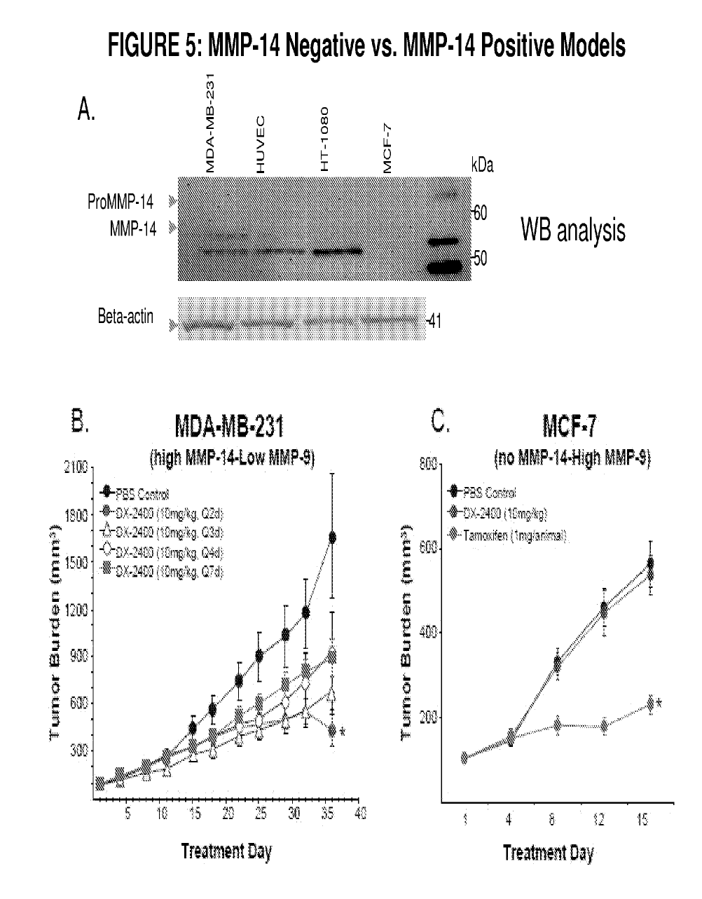

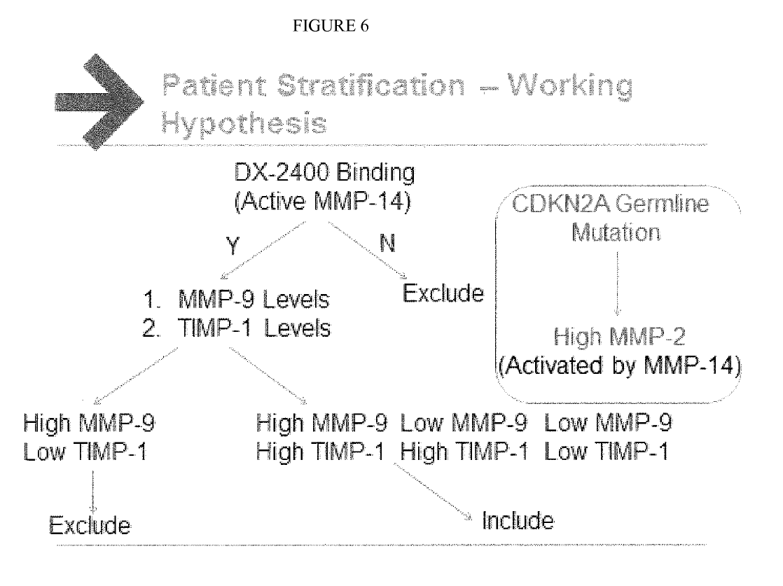

FIG. 5A shows MMP-14 expression in MDA-MB-231, HUVEC, HT-1080 and MCF-7 cells using a commercial anti-MMP-14 antibody (rabbit polyclonal antibody to MMP-14, Abcam, Cambridge, Mass.). These data show that the MCF-7 cells used for these experiments are negative for MMP-14, in contrast to MDA-MB-231.

FIGS. 5B and 5C show activity of DX-2400 in MDA-MB-231 and MCF-7 tumor xenograft models. As shown in FIG. 5B, DX-2400 inhibited tumor growth of MDA-MB-231 cells. The results seen with some treatments were statistically significant (see, e.g., DX-2400 10 mg/kg, Q2D). Consistent with the lack of MMP-14 expression in the MCF7 cells used for these experiments, DX-2400 (10 mg/kg, ip, qod) did not inhibit MCF-7 tumor growth after two weeks of treatment (FIG. 5C). In these MCF-7 cells, DX-2400 exhibited minimal tumor growth delay (37%) compared to Tamoxifen (83%) after 40 days of treatment. The slight response observed with DX-2400 may be attributed to stromal cells (MMP-14 positive) present in the tumor.

Western Blot Analysis.

To perform the Western blot experiments, whole cell protein extracts were prepared from cells using RIPA buffer. Equal amount of proteins (30 μg) was resolved by 4-12% SDS-PAGE and electroblotted to a PVDF membrane. The blot was probed with a rabbit polyclonal antibody to MMP-14 (Abcam, Cambridge, Mass.) followed by an HRP-conjugated goat anti-rabbit antibody (Thermo Fisher Scientific). Proteins were detected using a Super Signal West Femto Maximum Sensitivity Substrate (Thermo Fisher Scientific). The blot was subsequently stripped and reprobed with a mouse monoclonal antibody to β-actin (Abcam) followed by an HRP-conjugated goat anti-mouse antibody (Thermo Fisher Scientific).

### Example 3

### Exemplary MMP-14 Binding Antibodies

An exemplary MMP-14 antibody is M0038-F01. The variable domain sequences for M0038-F01 are:

| VH | |
|---|---|
| (SEQ ID NO: 13) | |
| 38F01 IgG | FR1--------------------------- CDR1- FR2----------- CDR2------- |
| | EVQLLESGGGLVQPGGSLRLSCAASGFTFS LYSMN WVRQAPGKGLEWVS SIYSSGGSTLY |
| 38F01 IgG | CDR2-- FR3----------------------------- CDR3-- FR4--------- |
| | ADSVKG RFTISRDNSKNTLYLQMNSLRAEDTAVYYCAR GRAFDI WGQGTMVTVSS |

CDR regions are in bold.

| VL | |
|---|---|
| (SEQ ID NO: 14) | |
| 38F01 IgG | FR1-------------------- CDR1------- FR2------------ CDR2--- |
| | DIQMTQSPSSLSAFVGDKVTITC RASQSVGTYLN WYQQKAGKAPELLIY ATSNLRS GVPS |
| 38F01 IgG | FR3------------------------- CDR3------ FR4------- |
| | RFSGSGSGTDFTLTINTLQPEDFATYYC QQSYSIPRFT FGPGTKVDIK |

CDR regions are in bold.

Another exemplary MMP-14 antibody is DX-2400. The variable domain sequences for DX-2400 are:

| VH: | |
|---|---|
| (SEQ ID NO: 15) | |
| DX-2400 | FR1--------------------------- CDR1- FR2----------- CDR2------- |
| | EVQLLESGGGLVQPGGSLRLSCAASGFTFS LYSMN WVRQAPGKGLEWVS SIYSSGGSTLY |
| DX-2400 | CDR2-- FR3----------------------------- CDR3-- FR4--------- |
| | ADSVKG RFTISRDNSKNTLYLQMNSLRAEDTAVYYCAR GRAFDI WGQGTMVTVSS |

CDR regions are in bold.

| VL: | |
|---|---|
| (SEQ ID NO: 16) | |
| DX-2400 | FR1-------------------- CDR1------- FR2------------ CDR2--- |
| | DIQMTQSPSSLSASVGDRVTITC RASQSVGTYLN WYQQKPGKAPKLLIY ATSNLRS GVPS |
| DX-2400 | FR3------------------------- CDR3------ FR4------- |
| | RFSGSGSGTDFTLTISSLQPEDFATYYC QQSYSIPRFT FGPGTKVDIK |

CDR regions are in bold.

Another exemplary MMP-14 antibody is M0033-H07. The variable domain sequences for M0033-H07 are:

| VH: | |
|---|---|
| (SEQ ID NO: 17) | |
| 33H07 IgG | FR1--------------------------- CDR1- FR2----------- CDR2------- |
| | EVQLLESGGGLVQPGGSLRLSCAASGFTFS VYGMV WVRQAPGKGLEWVS VISSSGGSTWY |
| 33H07 IgG | CDR2-- FR3----------------------------- CDR3------- FR4-------- |
| | ADSVKG RFTISRDNSKNTLYLQMNSLRAEDTALYYCAR PFSRRYGVFDY WGQGTLVTVSS |

CDR regions are in bold.

| VL: | |
|---|---|
| (SEQ ID NO: 18) | |
| 33H07 IgG | FR1-------------------- CDR1------- FR2------------ CDR2--- |
| | DIQMTQSPSSLSASVGDRVTITC RASQGIRNFLA WYQQKPGKVPKLLVF GASALQS |
| 33H07 IgG | FR3----------------------------- CDR3----- FR4------- |
| | GVPSRFSGSGSGTDFTLTISGLQPEDVATYYC QKYNGVPLT FGGGTKVEIK |

CDR regions are in bold.

Another exemplary MMP-14 antibody is DX-2410. The variable domain sequences for DX-2410 are:

| VH: | |
|---|---|
| (SEQ ID NO: 19) | |
| DX2410 | FR1--------------------------- CDR1- FR2----------- CDR2------- |
| | EVQLLESGGGLVQPGGSLRLSCAASGFTFS VYGMV WVRQAPGKGLEWVS VISSSGGSTWY |
| DX2410 | CDR2-- FR3----------------------------- CDR3------- FR4-------- |
| | ADSVKG RFTISRDNSKNTLYLQMNSLRAEDTAVYYCAR PFSRRYGVFDY WGQGTLVTVSS |

CDR regions are in bold.

| VL: | |
|---|---|
| (SEQ ID NO: 20) | |
| DX2410 | FR1-------------------- CDR1------- FR2------------ CDR2--- |
| | DIQMTQSPSSLSASVGDRVTITC RASQGIRNFLA WYQQKPGKVPKLLIY GASALQS |
| DX2410 | FR3----------------------------- CDR3----- FR4------- |
| | GVPSRFSGSGSGTDFTLTISSLQPEDVATYYC QKYNGVPLT FGGGTKVEIK |

CDR regions are in bold.

### Example 3

### Exemplary MMP-9 Binding Antibodies

An exemplary MMP-9 antibody is 539A-M0166-F10. The amino acid sequences of variable regions of 539A-M0166-F10 sFAB are as follows:

539A-M0166-F10 (phage/SFAB) VL leader + VL
(SEQ ID NO: 21)
FYSHSAQSELTQPPSASAAPGQRVTISCSGSSSNIGSNTVTWYQKLPGTAPKLLIYNNYERP
SGVPARFSGSKSGTSASLAISGLQSEDEADYYCATWDDSLIANYVFGSGTKVTVLGQPKANP
539A-M0166-F10 (phage/SFAB) VH leader + VH
(SEQ ID NO: 22)
MKKLLFAIPLVVPFVAQPAMAEVQLLESGGGLVQPGGSLRLSCAASGFTFSPYLMNWVRQA
PGKGLEWVSSIYSSGGGTGYADSVKGRFTISRDNSKNTLYLQMNSLRAEDTAVYYCARIYH
SSSGPFYGMDVWGQGTTVTVSSASTKGPSVFPLAPSSKS

Another exemplary MMP-9 antibody is 539A-M0240-B03. 539A-M0240-B03 is a selective inhibitor of MMP-9. 539A-M0240-B03 can decrease or inhibit the activity of human and mouse MMP-9. The sequences of the complementarity determining regions (CDRs) of 539A-M0240-B03 light chain (LC) and heavy chain (HC) are as follows:

LC CDR1:
(SEQ ID NO: 23)
TGTSSDVGGYNYVS
LC CDR2:
(SEQ ID NO: 24)
DVSKRPS
LC CDR3:
(SEQ ID NO: 25)
CSYAGSYTLV
HC CDR1:
(SEQ ID NO: 26)
TYQMV
HC CDR2:
(SEQ ID NO: 27)
VIYPSGGPTVYADSVKG
HC CDR3:
(SEQ ID NO: 28)
GEDYYDSSGPGAFDI

A protein containing the HC CDR sequences of 539A-M0240-B03 and the light chain sequence shown below can be used in the methods described herein. A protein containing the LC CDRs shown below and the HC CDRs of 539A-M0240-B03, or a protein containing the LC variable region (light V gene) shown below and the 539A-M0240-B03 HC CDRs can also be used in the methods described herein. The protein can include a constant region sequence, such as the constant region (LC-lambda1) shown below.

```
Light V gene = VL2_2e; J gene = JL3
(SEQ ID NO: 29)
        FR1-L CDR1-L FR2-L CDR2-L
QSALTQPRSVSGSPGQSVTISC TGTSSDVGGYNYVS WYQQHPGKAPKLMIY DVSKRPS GVPD
            FR3-L CDR3-L FR4-L
RFSGSKSGNTASLTISGLQAEDEADYYC CSYAGSYTLV FGGGTKLTVL
-------------------
LC-lambda1
(SEQ ID NO: 30)
GQPKAAPSVTLFPPSSEELQANKATLVCLISDFYPGAVTVAWKADSSPVKAGVETTTPSKQSNNKYAA
SSYLSLTPEQWKSHRSYSCQVTHEGSTVEKTVAPTECS
```

CDR regions are in bold.

The amino acid and nucleic acid sequences for another exemplary protein that can be used in the methods described herein are provided below. A protein containing the LC and HC CDRs shown below, or a protein containing the light chain and heavy chain variable regions (LV and HV, respectively) shown below can also be used in the methods described herein.

Light Chain
Ligh V gene = VL2_2e 2e.2.2/V1-3/DPL12
Light J gene = JL3

| | |
|---|---|
| Antibody A: | 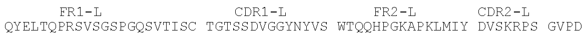 |
| Antibody A: | 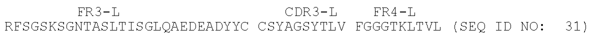 |

Heavy Chain
Heavy V gene: VH3_3-23 DP-47/V3-23
Heavy J gene: JH3

| | |
|---|---|
| Antibody A: | 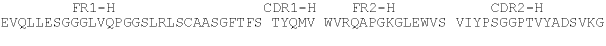 |
| Antibody A: | 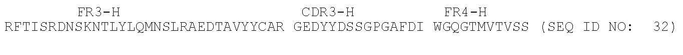 |

Light Variable
Antibody A-Light: Parental clone (sFab; IgG in pBh1 (f)) light variable

| | |
|---|---|
| | Q Y E L T Q P R S V S G S P G Q S V T I |
| Antibody A: | CAGTACGAATTGACTCAGCCTCGCTCAGTGTCCGGGTCTCCTGGACAGTCAGTCACCATC |
| Antibody A: | 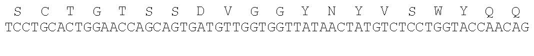 |
| Antibody A: | 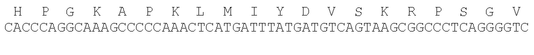 |
| | P D R F S G S K S G N T A S L T I S G L |
| Antibody A: | CCTGATCGCTTCTCTGGCTCCAAGTCTGGCAACACGGCCTCCCTGACCATCTCTGGGCTC |
| Antibody A: | 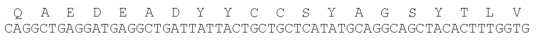 |
| | F G G G T K L T V L (SEQ ID NO: 33) |
| Antibody A: | TTCGGCGGAGGGACCAAGCTGACCGTCCTA (SEQ ID NO: 34) |

Heavy Variable
Antibody A-Heavy: Parental clone (sFab; IgG in pBh1 (f)) Heavy variable

| | |
|---|---|
| | E V Q L L E S G G G L V Q P G G S L R L |
| Antibody A: | GAAGTTCAATTGTTAGAGTCTGGTGGCGGTCTTGTTCAGCCTGGTGGTTCTTTACGTCTT |
| Antibody A: | 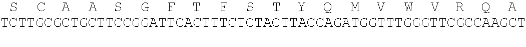 |
| Antibody A: | 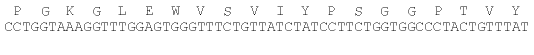 |
| Antibody A: | 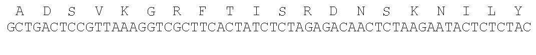 |
| Antibody A: | 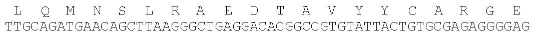 |
| Antibody A: | 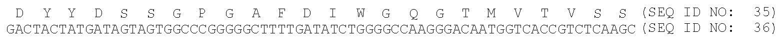 |

The amino acid and nucleic acid sequences for another exemplary protein that can be used in the methods described herein are provided below. A protein containing the LC and HC CDRs shown below, or a protein containing the light chain and heavy chain variable regions (LV and HV, respectively) shown below can also be used in the methods described herein. A protein containing the light chain and heavy chain (designated as LV+LC and HV+HC, respectively, below) sequences can also be used.

Light Chain
Light V gene = VL2_2e 2e.2.2/V1-3/DPL12
Light J gene = JL3

| | |
|---|---|
| Anti- body B: | 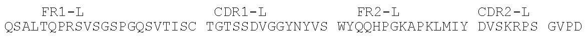 |
| Anti- body B: | 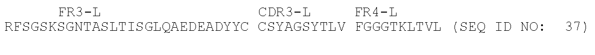 |

Heavy Chain
Heavy V gene: VH3_3-23 DP-47/V3-23
Heavy J gene: JH3

| | |
|---|---|
| Anti- body B: | 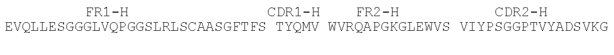 |
| Anti- body B: | 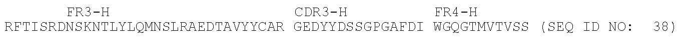 |

Light Variable
Antibody B-Light: Germlined, codon optimized in GS vector

| | |
|---|---|
| Anti- | CAGAGCGCCCTGACCCAGCCCAGAAGCGTGTCCGGCAGCCCAGGCCAGAGCGTGACCATC |
| body | Q S A L T Q P R S V S G S P G Q S V T I |
| B: | |
| Anti- body B: | 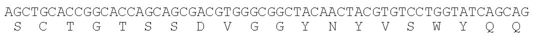 |
| Anti- body B: | 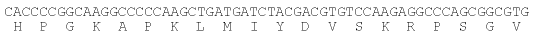 |
| Anti- | CCCGACAGGTTCAGCGGCAGCAAGAGCGCAACACCGCCAGCCTGACCATCTCCGGACTG |
| body | P D R F S G S K S G N T A S L T I S G L |
| B: | |
| Anti- body B: | 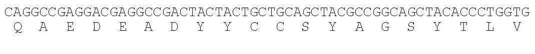 |
| Anti- | TTCGGCGGAGGGACCAAGCTGACCGTGCTG (SEQ ID NO: 39) |
| body | F G G G T K L T V L (SEQ ID NO: 40) |
| B: | |

Heavy Variable
Antibody B-Heavy: Germlined, codon optimized in GS vector

| | |
|---|---|
| Anti- | GAGGTGCAATTGCTGGAAAGCGGCGGAGGACTGGTGCAGCCAGGCGGCAGCCTGAGGCTG |
| body | E V Q L L E S G G G L V Q P G G S L R L |
| B: | |
| Anti- body B: | 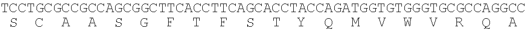 |
| Anti- body B: | 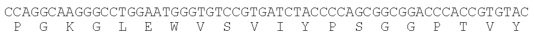 |
| Anti- body B: | 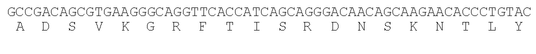 |
| Anti- body B: | 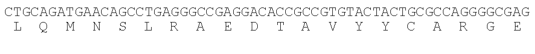 |
| Anti- body B: | 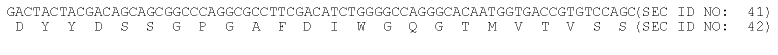 |

>Antibody B: LV + LC dna
CAGAGCGCCCTGACCCAGCCCAGAAGCGTGTCCGGCAGCCCAGGCCAGAGCGTGACCATCAGCTGCACCGGCACCAGCAGCGACGTGGGCGGCTACAACTAC
GTGTCCTGGTATCAGCAGCACCCCGGCAAGGCCCCCAAGCTGATGATCTACGACGTGTCCAAGAGGCCCAGCGGCGTGCCCGACAGGTTCAGCGGCAGCAAGA
GCGGCAACAACCGTGCTGGGCCAGCCCAAGGCTGCCCCAGCGTGACCCTGTTCCCCCCCAGCAGCGAGGAACTGCAGGCCAACAAGGCCACACTGGTGTGCCT
GATCAGCGACTTCTACCCAGGCGCCGTGACCGTGGCCTGGAAGGCCGACAGCAGCCCCGTGAAGGCCGGCGTGGAGACAACCACCCCCAGCAAGCAGAGCAA
CAACAAGTACGCCGCCAGCAGCTACCTGAGCCTGACCCCCGAGCAGTGGAAGTCCCACAGGTCCTACAGCTGCCAGGTGACCCACGAGGGCAGCACCGT
GGAGAAAACCGTGGCCCCCACCGAGTGTAGCTGATGA (SEQ ID NO: 43)
>Antibody B: HV + HC dna
GAGGTGCAATTGCTGGAAAGCGGCGGAGGACTGGTGCAGCCAGGCGGCAGCCTGAGGCTGTCCTGCGCCGCCAGCGGCTTCACCTTCAGCACCTACCAGATG
GTGTGGGTGCGCCAGGCCCCAGGCAAGGGCCTGGAATGGGTGTCCGTGATCTACCCCAGCGGCGGACCCACCGTGTACGCCGACAGCGTGAAGGGCAGGTTC
ACCATCAGCAGGGACAACAGCAAGAACACCCTGTACCTGCAGATGAACAGCCTGAGGGCCGAGGACACCGCCGTGTACTACTGCGCCAGGGGCGAGGACTA
CTACGACAGCAGCGGCCCAGGCGCCTTCGACATCTGGGGCCAGGGCACAATGGTGACCGTGTCCAGCGCCAGCACCAAGGGCCCCAGCGTGTTCCCGCTAGC
ACCTTCCTCCAAGTCCACCTCTGGCGGCACCGCCGCTCTGGGCTGCCTGGTGAAGGACTACTTCCCTGAGCCTGTGACCGTGAGCTGGAACTCTGGCGCCC
TGACCTCCGGCGTGCATACCTTCCCTGCCGTGCTGCAGTCCTCCGGCCTGTACTCCCTGTCCTCCGTGGTGACAGTGCCTTCCTCCTCCCTGGGCACCCA
GACCTACATCTGCAACGTGAACCACAAGCCTTCCAACACCAAGGTGGACAAGCGGGTGGAGCCTAAGTCCTGCGACAAGACCCACACCTGCCCTCCCTGC
CCTGCCCCTGAGCTGCTGGGCGGACCCTCCGTGTTCCTGTTCCCTCCTAAGCCTAAGGACACCCTGATGATCTCCCGGACCCCTGAGGTGACCTGCGTGGT
GGTGGACGTGTCCCACGAGGACCCAGAGGTGAAGTTTAATTGTATGTGGACGGCGTGGAGGTCCACAACGCCAAGACCAAGCCTCGGGAGGAACAGTACAA
CTCCACCTACCGGGTGGTGTCCGTGCTGACCGTGCTGCACCAGGACTGGCTGAACGGCAAGGAATACAAGTGCAAAGTCTCCAACAAGGCCCTGCCTGCCC
CCATCGAGAAAACCATCTCCAAGGCCAAGGGCCAGCCTCGCGAGCCTCAGGTGTACACCCTGCCTCCTAGCCGGGAGGAAATGACCAAGAACCAGGTGTC
CCTGACCTGTCTGGTGAAGGGCTTCTACCCTTCCGATATCGCCGTGGAGTGGGAGTCCAAACGCCGCCTGAGAACAACTACAAGACCACCCCTCCTGTG
CTGGACTCCGACGGCTCCTTCTTCCTGTACTCCAAGCTGACCGTGGACAAGTCCCGGTGGCAGCAGGGCAACGTGTTCTCCTGCTCC
GTGATGCACGAGGCCCTGCACAACCACTACACCCAGAAGTCCCTGTCCCTGAGCCCTGGCAAGTGA (SEQ ID NO: 44)
>Antibody B: LV + LC aa
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGS
YTLVFGGGTKLTVLGQPKAAPSVTLFPPSSEELQANKATLVCLISDFYPGAVTVAWKADSSPVKAGVETTTPSKQSNNKYAASSYLSLTPEQ
WKSHRSYSCQVTHEGSTVEKTVAPTECSss (SEQ ID NO: 45)
>Antibody B: HV + HC aa
EVQLLESGGGLVQPGGSLRLSCAASGFTFSTYQMVWVRQAPGKGLEWVSVIYPSGGPTVYADSVKGRFTISRDNSKNTLYLQMNSLRAEDTAVYYCARG
EDYYDSSGPGAFDIWGQGTMVTVSSASTKGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGKs (SEQ ID NO: 46)

### REFERENCES

The contents of all cited references including literature references, issued patents, published or non-published patent applications cited throughout this application are hereby expressly incorporated by reference in their entireties. In case of conflict, the present application, including any definitions herein, will control.

### EQUIVALENTS

A number of embodiments of the invention have been described. Nevertheless, it will be understood that various modifications may be made without departing from the spirit and scope of the invention. Accordingly, other embodiments are within the scope of the following claims.

1.-12. (canceled)

13. A method of identifying a subject who may benefit from administration of an MMP-14 inhibitor to treat cancer, the method comprising
obtaining a sample from a subject having cancer, and
determining an expression and/or protein activity ratio of MMP-9/TIMP or MMP-2/TIMP in the sample,
wherein an expression and/or protein activity ratio of MMP-9/TIMP that is less than or equal to 1 or an expression and/or protein activity ratio of MMP-2/TIMP that is greater than 1 is indicative that the subject will benefit from treatment with the MMP-14 inhibitor.

14. The method of claim 13, further comprising evaluating MMP-2 or MMP-9 expression and/or protein activity in the sample.

15. The method of claim 13, further comprising evaluating TIMP expression and/or protein activity in the sample.

16. The method of claim 15, wherein the TIMP is TIMP-1.

17. The method of claim 13, wherein the cancer is selected from the group consisting of osteotropic cancer, breast cancer, lung cancer, melanoma, pancreatic cancer, colon cancer, and prostate cancer.

18. The method of claim 13, wherein the sample is a tumor biopsy.

19. The method of claim 13, wherein the MMP-14 inhibitor is DX-2400.

20. A method of treating cancer in a subject, the method comprising
identifying a subject who may benefit from administration of an MMP-14 inhibitor to treat cancer by the method of claim 13, and
administering an MMP-14 inhibitor to the subject.

21. A method of selecting a therapy for cancer for a subject, the method comprising
obtaining a sample from a subject having cancer, and
determining an expression and/or protein activity ratio of MMP-9/TIMP or MMP-2/TIMP in the sample,
wherein an MMP-14 inhibitor is selected as a therapy when an expression and/or protein activity ratio of MMP-9/TIMP is less than or equal to 1 or an expression and/or protein activity ratio of MMP-2/TIMP is greater than 1.

22. The method of claim 21, further comprising evaluating MMP-2 or MMP-9 expression and/or protein activity in the sample.

23. The method of claim 21, further comprising evaluating TIMP expression and/or protein activity in the sample.

24. The method of claim 23, wherein the TIMP is TIMP-1.

25. The method of claim 21, wherein the cancer is selected from the group consisting of osteotropic cancer, breast cancer, lung cancer, melanoma, pancreatic cancer, colon cancer, and prostate cancer.

26. The method of claim 21, wherein the sample is a tumor biopsy.

27. The method of claim 21, wherein the MMP-14 inhibitor is DX-2400.

28. A method of monitoring the progress of a therapy for cancer in a subject, the method comprising
obtaining a sample from a subject having cancer, and
determining an expression and/or protein activity ratio of MMP-9/TIMP or MMP-2/TIMP in the sample.

29. The method of claim 28, further comprising evaluating MMP-2 or MMP-9 expression and/or protein activity in the sample.

30. The method of claim 28, further comprising evaluating TIMP expression and/or protein activity in the sample.

31. The method of claim 30, wherein the TIMP is TIMP-1.

32. The method of claim 28, wherein the cancer is selected from the group consisting of osteotropic cancer, breast cancer, lung cancer, melanoma, pancreatic cancer, colon cancer, and prostate cancer.

33. The method of claim 28, wherein the sample is a tumor biopsy.

34. The method of claim 28, wherein the therapy comprises an MMP-14 inhibitor.

35. A method of identifying a subject who may benefit from administration of an MMP-14 inhibitor to treat cancer, the method comprising
obtaining a sample from a subject having cancer, and
determining the presence of a mutation in the cyclin-dependent kinase inhibitor 2A (CDKN2A) gene in the sample,
wherein the presence of the mutation is indicative that the subject will benefit from treatment with the MMP-14 inhibitor.

36. The method of claim 35, wherein the cancer is selected from the group consisting of skin cancer, gastric cancer, esophageal cancer, and pancreatic cancer.

37. An assay for determining if a subject having cancer will benefit from treatment with an MMP-14 inhibitor, the assay comprising a probe that binds to and detects MMP-9 and/or a probe that binds to and detects MMP-2, and a probe which binds and detects TIMP.


SEQUENCE LISTING (55 sequences)

SEQ ID NO: 1 - protein, 582 aa, Homo sapiens
MSPAPRPPRCLLLPLLTLGTALASLGSAQSSSFSPEAWLQQYGYLPPGDLRTHTQRSPQSLSAAIAAMQKFYGLQVTGKADADTMKAMRRPRCGVPDKFGAEIKANVRRKRYAIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSGGRPDEGTEEETEVIIIEVDEEGGGAVSAAAVVLPVLLLLLVLAVGLAVFFFRRHGTPRRLLYCQRSLLDKV

SEQ ID NO: 2 - protein, 582 aa, Mus musculus
MSPAPRPSRSLLLPLLTLGTALASLGWAQGSNFSPEAWLQQYGYLPPGDLRTHTQRSPQSLSAAIAAMQKFYGLQVTGKADLATMMAMRRPRCGVPDKFGTEIKANVRRKRYAIQGLKWQHNEITFCIQNYTPKVGEYATFEAIRKAFRVWESATPLRFREVPYAYIREGHEKQADIMILFAEGFHGDSTPFDGEGGFLAHAYFPGPNIGGDTHFDSAEPWTVQNEDLNGNDIFLVAVHELGHALGLEHSNDPSAIMSPFYQWMDTENFVLPDDDRRGIQQLYGSKSGSPTKMPPQPRTTSRPSVPDKPKNPAYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEEFRAVDSEYPKNIKVWEGIPESPRGSFMGSDEVFTYFYKGNKYWKFNNQKLKVEPGYPKSALRDWMGCPSGRRPDEGTEEETEVIIIEVDEEGSGAVSAAAVVLPVLLLLLVLAVGLAVFFFRRHGTPKRLLYCQRSLLDKV

SEQ ID NO: 3 - DNA, 3437 bp, Homo sapiens
aagttcagtgcctaccgaagacaaaggcgccccgagggagtggcggtgcgaccccagggcgtgggcccggccgcggagcccacactgcccggctgacccggtggtctcggaccatgtctcccgccccaagacccccccgttgtctcctgctccccctgctcacgctcggcaccgcgctcgcctccctcggctcggcccaaagcagcagcttcagccccgaagcctggctacagcaatatggctacctgcctcccggggacctacgtacccacacacagcgctcaccccagtcactctcagcggccatcgctgccatgcagaagttttacggcttgcaagtaacaggcaaagctgatgcagacaccatgaaggccatgaggcgcccccgatgtggtgttccagacaagtttggggctgagatcaaggccaatgttcgaaggaagcgctacgccatccagggtctcaaatggcaacataatgaaatcactttctgcatccagaattacacccccaaggtgggcgagtatgccacatacgaggccattcgcaaggcgttccgcgtgtgggagagtgccacaccactgcgcttccgcgaggtgccctatgcctacatccgtgagggccatgagaagcaggccgacatcatgatcttctttgccgagggcttccatggcgacagcacgcccttcgatggtgagggcggcttcctggcccatgcctacttcccaggccccaacattggaggagacacccactttgactctgccgagccttggactgtcaggaatgaggatctgaatggaaatgacatcttcctggtggctgtgcacgagctgggccatgccctggggctcgagcattccagtgacccctcggccatcatggcacccttttaccagtggatggacacggagaattttgtgctgcccgatgatgaccgccggggcatccagcaactttatgggggtgagtcagggttccccaccaagatgccccctcaacccaggactacctcccggccttctgttcctgataaacccaaaaaccccacctatgggcccaacatctgtgacgggaactttgacaccgtggccatgctccgaggggagatgtttgtcttcaaggagcgctggttctggcgggtgaggaataaccaagtgatggatggatacccaatgcccattggccagttctggcggggcctgcctgcgtccatcaacactgcctacgagaggaaggatggcaaattcgtcttcttcaaaggagacaagcattgggtgtttgatgaggcgtccctggaacctggctaccccaagcacattaaggagctgggccgagggctgcctaccgacaagattgatgctgctctcttctggatgcccaatggaaagacctacttcttccgtggaaacaagtactaccgtttcaacgaagagctcagggcagtggatagcgagtaccccaagaacatcaaagtctgggaagggatccctgagtctcccagagggtcattcatgggcagcgatgaagtcttcacttacttctacaaggggaacaaatactggaaattcaacaaccagaagctgaaggtagaaccgggctaccccaagtcagccctgagggactggatgggctgcccatcgggaggccggccggatgaggggactgaggaggagacggaggtgatcatcattgaggtggacgaggagggcggcggggcggtgagcgcggctgccgtggtgctgcccgtgctgctgctgctcctggtgctggcggtgggccttgcagtcttcttcttcagacgccatgggacccccaggcgactgctctactgccagcgttccctgctggacaaggtctgacgcccaccgccggcccgcccactcctaccacaaggactttgcctctgaaggccagtggcagcaggtggtggtgggtgggctgctcccatcgtcccgagccccctccccgcagcctccttgcttctctctgtcccctggctggcctccttcaccctgaccgcctccctccctcctgccccggcattgcatcttccctagataggtcccctgagggctgagtgggagggcggccctttccagcctctgcccctcaggggaaccctgtagctttgtgtctgtccagccccatctgaatgtgttgggggctctgcacttgaaggcaggaccctcagacctcgctggtaaaggtcaaatggggtcatctgctccttttccatcccctgacataccttaacctctgaactctgacctcaggaggctctgggcactccagccctgaaagccccaggtgtacccaattggcagcctctcactactctttctggctaaaaggaatctaatcttgttgagggtagagaccctgagacagtgtgagggggtggggactgccaagccaccctaagaccttgggaggaaaactcagagagggtcttcgttgctcagtcagtcaagttcctcggagatctgcctctgcctcacctaccccagggaacttccaaggaaggagcctgagccactggggactaagtgggcagaagaaacccttggcagccctgtgcctctcgaatgttagccttggatggggctttcacagttagaagagctgaaaccaggggtgcagctgtcaggtagggtggggccggtgggagaggcccgggtcagagccctgggggtgagcctgaaggccacagagaaagaaccttgcccaaactcaggcagctggggctgaggcccaaaggcagaacagccagagggggcaggaggggaccaaaaaggaaaatgaggacgtgcagcagcattggaaggctggggccgggcaggccaggccaagccaagcagggggccacagggtgggctgtggagctctcaggaagggccctgaggaaggcacacttgctcctgttggtccctgtccttgctgcccaggcagcgtggaggggaagggtagggcagccagagaaaggagcagagaaggcacacaaacgaggaatgaggggcttcacgagaggccacagggcctggctggccacgctgtcccggcctgctcaccatctcagtgaggggcaggagctggggctcgcttaggctgggtccacgcttccctggtgccagcacccctcaagcctgtctcaccagtggcctgccctctcgctcccccacccagcccacccattgaagtctccttgggccaccaaaggtggtggccatggtaccggggacttgggagagtgagacccagtggagggagcaagaggagagggatgtcgggggggtggggcacggggtaggggaaatggggtgaacggtgctggcagttcggctagatttctgtcttgtttgtttttttgttttgtttaatgtatatttttattataattattatatatgaattccaaaaaaaaaaaaaaaaaaaaa

SEQ ID NO: 4 - DNA, 2583 bp, Mus musculus
caaaggagagcagagagggcttccaactcagttcgccgactaagcagaagaaagatcaaaaacggaaaagagaagagcaaacagacatttccaggagcaattccctcacctccaagccgaccgcgctctaggaatccacattccgttcctttagaagacaaaggcgccccaagagaggcggcgcgaccccagggcgtgggccccgccgcggagcccgcaccgcccggcgccccgacgccggggaccatgtctcccgcccctcgaccctcccgcagcctcctgctccccctgctcacgcttggcacggcgctcgcctccctcggctgggcccaaggcagcaacttcagccccgaagcctggctgcagcagtatggctacctacctccaggggacctgcgtacccacacacaacgctcaccccagtcactctcagctgccattgccgccatgcaaaagttctatggtttacaagtgacaggcaaggctgatttggcaaccatgatggccatgaggcgccctcgctgtggtgttccggataagtttgggactgagatcaaggccaatgttcggaggaagcgctatgccattcagggcctcaagtggcagcataatgagatcactttctgcattcagaattacacccctaaggtgggcgagtatgccacattcgaggccattcggaaggccttccgagtatgggagagtgccacgccactgcgcttccgagaagtgccctatgcctacatccgggagggacatgagaagcaggctgacatcatgatcttatttgctgagggtttccacggcgacagtacaccctttgatggtgaaggagggttcctggctcatgcctacttcccaggccccaatattggaggggatacccactttgattctgccgagccctggactgtccaaaatgaggatctaaatgggaatgacatcttcttggtggctgtgcatgagttggggcatgccctaggcctggaacattctaacgatccctccgccatcatgtcccccttttaccagtggatggacacagagaacttcgtgttgcctgatgacgatcgccgtggcatccagcaactttatggaagcaagtcagggtcacccacaaagatgccccctcaacccagaactacctctcggccctctgtcccagataagcccaaaaaccccgcctatgggcccaacatctgtgacgggaactttgacaccgtggccatgctccgaggagagatgtttgtcttcaaggagcgatggttctggcgggtgaggaataaccaagtgatggatggatacccaatgcccattggccaattctggaggggcctgcctgcatccatcaatactgcctacgaaaggaaggatggcaaatttgtcttcttcaaaggagataagcactgggtgtttgacgaagcctccctggaacccgggtaccccaagcacattaaggagcttggccgagggctgcccacggacaagatcgatgcagctctcttctggatgcccaatgggaagacctacttcttccggggcaataagtactaccggttcaatgaagaattcagggcagtggacagcgagtaccctaaaaacatcaaagtctgggaaggaatccctgaatctcccagggggtcattcatgggcagtgatgaagtcttcacatacttctacaagggaaacaaatactggaagttcaacaaccagaagctgaaggtagagccagggtaccccaagtcagctctgcgggactggatgggctgcccttcggggcgccggcccgatgaggggactgaggaggagacagaggtgatcatcattgaggtggatgaggagggcagtggagctgtgagtgcggccgccgtggtcctgccggtactactgctgctcctggtactggcagtgggcctcgctgtcttcttcttcagacgccatgggacgcccaagcgactgctttactgccagcgttcgctgctggacaaggtctgacccccaccactggcccacccgcttctaccacaaggactttgcctctgaaggccagtggctacaggtggtagcaggtgggctgctctcacccgtcctgggctccctccctccagcctcccttctcagtccctaattggcctctcccaccctcaccccagcattgcttcatccataagtgggtcccttgagggctgagcagaagacggtcggcctctggccctcaagggaatctcacagctcagtgtgtgttcagccctagttgaatgttgtcaaggctcttattgaaggcaagaccctctgaccttataggcaacggccaaatggggtcatctgcttcttttccatccccctaactacataccttaaatctctgaactctgacctcaggaggctctgggcatatgagccctatatgtaccaagtgtacctagttggctgcctcccgccactctgactaaaaggaatcttaagagtgtacatttggaggtggaaagattgttcagtttaccctaaagactttgataagaaagagaaagaaagaaagaaagaaagaaagaaagaaagaaagaaagaaagaaaaaaaaaaaa

SEQ ID NO: 5 - protein, 660 aa, Homo sapiens
MEALMARGALTGPLRALCLLGCLLSHAAAAPSPIIKFPGDVAPKTDKELAVQYLNTFYGCPKESCNLFVLKDTLKKMQKFFGLPQTGDLDQNTIETMRKPRCGNPDVANYNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 6 - protein, 662 aa, Mus musculus
MEARVAWGALAGPLRVLCVLCCLLGRAIAAPSPIIKFPGDVAPKTDKELAVQYLNTFYGCPKESCNLFVLKDTLKKMQKFFGLPQTGDLDQNTIETMRKPRCGNPDVANYNFFPRKPKWDKNQITYRIIGYTPDLDPETVDDAFARALKVWSDVTPLRFSRIHDGEADIMINFGRWEHGDGYPFDGKDGLLAHAFAPGTGVGGDSHFDDDELWTLGEGQVVRVKYGNADGEYCKFPFLFNGREYSSCTDTGRSDGFLWCSTTYNFEKDGKYGFCPHEALFTMGGNADGQPCKFPFRFQGTSYNSCTTEGRTDGYRWCGTTEDYDRDKKYGFCPETAMSTVGGNSEGAPCVFPFTFLGNKYESCTSAGRNDGKVWCATTTNYDDDRKWGFCPDQGYSLFLVAAHEFGHAMGLEHSQDPGALMAPIYTYTKNFRLSHDDIKGIQELYGPSPDADTDTGTGPTPTLGPVTPEICKQDIVFDGIAQIRGEIFFFKDRFIWRTVTPRDKPTGPLLVATFWPELPEKIDAVYEAPQEEKAVFFAGNEYWVYSASTLERGYPKPLTSLGLPPDVQQVDAAFNWSKNKKTYIFAGDKFWRYNEVKKKMDPGFPKLIADSWNAIPDNLDAVVDLQGGGHSYFFKGAYYLKLENQSLKSVKFGSIKSDWLGC

SEQ ID NO: 7 - DNA, 3546 bp, Homo sapiens
gcggctgccctcccttgtttccgctgcatccagacttcctcaggcggtggctggaggctgcgcatctggggctttaaacatacaaagggattgccaggacctgcggcggcggcggcggcggcgggggctggggcgcgggggccggaccatgagccgctgagccgggcaaaccccaggccaccgagccagcggaccctcggagcgcagccctgcgccgcggagcaggctccaaccaggcggcgaggcggccacacgcaccgagccagcgacccccgggcgacgcgcggggccagggagcgctacgatggaggcgctaatggcccggggcgcgctcacgggtcccctgagggcgctctgtctcctgggctgcctgctgagccacgccgccgccgcgccgtcgcccatcatcaagttccccggcgatgtcgcccccaaaacggacaaagagttggcagtgcaatacctgaacaccttctatggctgccccaaggagagctgcaacctgtttgtgctgaaggacacactaaagaagatgcagaagttctttggactgccccagacaggtgatcttgaccagaataccatcgagaccatgcggaagccacgctgcggcaacccagatgtggccaactacaacttcttccctcgcaagcccaagtgggacaagaaccagatcacatacaggatcattggctacacacctgatctggacccagagacagtggatgatgcctttgctcgtgccttccaagtctggagcgatgtgaccccactgcggttttctcgaatccatgatggagaggcagacatcatgatcaactttggccgctgggagcatggcgatggatacccctttgacggtaaggacggactcctggctcatgccttcgccccaggcactggtgttgggggagactcccattttgatgacgatgagctatggaccttgggagaaggccaagtggtccgtgtgaagtatgggaacgccgatggggagtactgcaagttccccttcttgttcaatggcaaggagtacaacagctgcactgataccggccgcagcgatggcttcctctggtgctccaccacctacaactttgagaaggatggcaagtacggcttctgtccccatgaagccctgttcaccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggcttctgccctgagaccgccatgtccactgttggtgggaactcagaaggtgccccctgtgtcttccccttcactttcctgggcaacaaatatgagagctgcaccagcgccggccgcagtgacggaaagatgtggtgtgcgaccacagccaactacgatgatgaccgcaagtggggcttctgccctgaccaagggtacagcctgttcctcgtggcagcccacgagtttggccacgccatggggctggagcactcccaagaccctggggccctgatggcacccatttacacctacaccaagaacttccgtctgtcccaggatgacatcaagggcattcaggagctctatggggcctctcctgacattgaccttggcaccggccccacccccacgctgggccctgtcactcctgagatctgcaaacaggacattgtatttgatggcatcgctcagatccgtggtgagatcttcttcttcaaggaccggttcatttggcggactgtgacgccacgtgacaagcccatggggcccctgctggtggccacattctggcctgagctcccggaaaagattgatgcggtatacgaggccccacaggaggagaaggctgtgttctttgcagggaatgaatactggatctactcagccagcaccctggagcgagggtaccccaagccactgaccagcctgggactgccccctgatgtccagcgagtggatgccgcctttaactggagcaaaaacaagaagacatacatctttgctggagacaaattctggagatacaatgaggtgaagaagaaaatggatcctggcttccccaagctcatcgcagatgcctggaatgccatccccgataacctggatgccgtcgtggacctgcagggcggcggtcacagctacttcttcaagggtgcctattacctgaagctggagaaccaaagtctgaagagcgtgaagtttggaagcatcaaatccgactggctaggctgctgagctggccctggctcccacaggcccttcctctccactgccttcgatacaccgggcctggagaactagagaaggacccggaggggcctggcagccgtgccttcagctctacagctaatcagcattctcactcctacctggtaatttaagattccagagagtggctcctcccggtgcccaagaatagatgctgactgtactcctcccaggcgccccttccccctccaatcccaccaaccctcagagccacccctaaagagatactttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcagtgggacagggcatggccaggtggccactccagacccctggcttttcactgctggctgccttagaacctttcttacattagcagtttgctttgtatgcactttgtttttttctttgggtcttgttttttttttccacttagaaattgcatttcctgacagaaggactcaggttgtctgaagtcactgcacagtgcatctcagcccacatagtgatggttcccctgttcactctacttagcatgtccctaccgagtctcttctccactggatggaggaaaaccaagccgtggcttcccgctcagccctccctgcccctcccttcaaccattccccatgggaaatgtcaacaagtatgaataaagacacctactgagtggccgtgtttgccatctgttttagcagagcctagacaagggccacagacccagccagaagcggaaacttaaaaagtccgaatctctgctccctgcagggcacaggtgatggtgtctgctggaaaggtcagagcttccaaagtaaacagcaagagaacctcagggagagtaagctctagtccctctgtcctgtagaaagagccctgaagaatcagcaattttgttgctttattgtggcatctgttcgaggtttgcttcctctttaagtctgtttcttcattagcaatcatatcagttttaatgctactactaacaatgaacagtaacaataatatccccctcaattaatagagtgctttctatgtgcaaggcacttttcacgtgtcacctattttaacctttccaaccacataaataaaaaaggccattattagttgaatcttattgatgaagagaaaaaaaaaaa

SEQ ID NO: 8 - DNA, 3070 bp, Mus musculus
ccagccggccacatctggcgtctgcccgcccttgtttccgctgcatccagacttccctggtggctggaggctctgtgtgcatccaggagtttagatatacaaagggattgccaggacctgcaagcacccgcggcagtggtgtgtattgggacgtgggaccccgttatgagctcctgagccccgagaagcagaggcagtagagtaaggggatcgccgtgcagggcaggcgccagccgggcggaccccagggcacagccagagacctcagggtgacacgcggagcccgggagcgcaacgatggaggcacgagtggcctggggagcgctggccggacctctgcgggttctctgcgtcctgtgctgcctgttgggccgcgccatcgctgcaccatcgcccatcatcaagttccccggcgatgtcgcccctaaaacagacaaagagttggcagtgcaatacctgaacactttctatggctgccccaaggagagttgcaacctctttgtgctgaaagataccctcaagaagatgcagaagttctttgggctgccccagacaggtgaccttgaccagaacaccatcgagaccatgcggaagccaagatgtggcaacccagatgtggccaactacaacttcttcccccgcaagcccaagtgggacaagaaccagatcacatacaggatcattggttacacacctgacctggaccctgaaaccgtggatgatgcttttgctcgggccttaaaagtatggagcgacgtcactccgctgcgcttttctcgaatccatgatggggaggctgacatcatgatcaactttggacgctgggagcatggagatggatacccatttgatggcaaggatggactcctggcacatgcctttgccccgggcactggtgttgggggagattctcactttgatgatgatgagctgtggaccctgggagaaggacaagtggtccgcgtaaagtatgggaacgctgatggcgagtactgcaagttccccttcctgttcaacggtcgggaatacagcagctgtacagacactggtcgcagtgatggcttcctctggtgctccaccacatacaactttgagaaggatggcaagtatggcttctgcccccatgaagccttgtttaccatgggtggcaatgcagatggacagccctgcaagttcccgttccgcttccagggcacctcctacaacagctgtaccaccgagggccgcaccgatggctaccgctggtgtggcaccaccgaggactatgaccgggataagaagtatggattctgtcccgagaccgctatgtccactgtgggtggaaattcagaaggtgccccatgtgtcttccccttcactttcctgggcaacaagtatgagagctgcaccagcgccggccgcaacgatggcaaggtgtggtgtgcgaccacaaccaactacgatgatgaccggaagtggggcttctgtcctgaccaaggatatagcctattcctcgtggcagcccatgagttcggccatgccatggggctggaacactctcaggaccctggagctctgatggccccgatctacacctacaccaagaacttccgattatcccatgatgacatcaaggggatccaggagctctatgggccctcccccgatgctgatactgacactggtactggccccacaccaacactgggacctgtcactccggagatctgcaaacaggacattgtctttgatggcatcgctcagatccgtggtgagatcttcttcttcaaggaccggtttatttggcggacagtgacaccacgtgacaagcccacaggtcccttgctggtggccacattctggcctgagctcccagaaaagattgacgctgtgtatgaggccccacaggaggagaaggctgtgttcttcgcagggaatgagtactgggtctattctgctagtactctggagcgaggataccccaagccactgaccagcctggggttgccccctgatgtccagcaagtagatgctgcctttaactggagtaagaacaagaagacatacatctttgcaggagacaagttctggagatacaatgaagtgaagaagaaaatggaccccggtttccctaagctcatcgcagactcctggaatgccatccctgataacctggatgccgtcgtggacctgcagggtggtggtcatagctacttcttcaagggtgcttattacctgaagctggagaaccaaagtctcaagagcgtgaagtttggaagcatcaaatcagactggctgggctgctgagctggccctgttcccacgggccctatcatcttcatcgctgcacaccaggtgaaggatgtgaagcagcctggcggctctgtcctcctctgtagttaaccagccttctccttcacctggtgacttcagatttaagagggtggcttctttttgtgcccaaagaaaggtgctgactgtaccctcccgggtgctgcttctccttcctgcccaccctaggggatgcttggatatttgcaatgcagccctcctctgggctgccctggtgctccactcttctggttcttcaacatctatgacctttttatggctttcagcactctcagagttaatagagactggcttaggagggcactggtggccctgttaacagcctggcatggggcagtggggtacaggtgtgccaaggtggaaatcagagacacctggtttcaccctttctgctgcccagacacctgcaccaccttaactgttgcttttgtatgcccttcgctcgtttccttcaaccttttcagttttccactccactgcatttcctgcccaaaggactcgggttgtctgacatcgctgcatgatgcatctcagcccgcctagtgatggttcccctcctcactctgtgcagatcatgcccagtcacttcctccactggatggaggagaaccaagtcagtggcttcctgctcagccttcttgcttctccctttaacagttccccatgggaaatggcaaacaagtataaataaagacacccattgagtgacaaaaaaaaaaaaaaaaaaaaaaaaaaaaaa

SEQ ID NO: 9 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 10 - protein, 730 aa, Mus musculus
MSPWQPLLLALLAFGCSSAAPYQRQPTFVVFPKDLKTSNLTDTQLAEAYLYRYGYTRAAQMMGEKQSLRPALLMLQKQLSLPQTGELDSQTLKAIRTPRCGVPDVGRFQTFKGLKWDHHNITYWIQNYSEDLPRDMIDDAFARAFAVWGEVAPLTFTRVYGPEADIVIQFGVAEHGDGYPFDGKDGLLAHAFPPGAGVQGDAHFDDDELWSLGKGVVIPTYYGNSNGAPCHFPFTFEGRSYSACTTDGRNDGTPWCSTTADYDKDGKFGFCPSERLYTEHGNGEGKPCVFPFIFEGRSYSACTTKGRSDGYRWCATTANYDQDKLYGFCPTRVDATVVGGNSAGELCVFPFVFLGKQYSSCTSDGRRDGRLWCATTSNFDTDKKWGFCPDQGYSLFLVAAHEFGHALGLDHSSVPEALMYPLYSYLEGFPLNKDDIDGIQYLYGRGSKPDPRPPATTTTEPQPTAPPTMCPTIPPTAYPTVGPTVGPTGAPSPGPTSSPSPGPTGAPSPGPTAPPTAGSSEASTESLSPADNPCNVDVFDAIAEIQGALHFFKDGWYWKFLNHRGSPLQGPFLTARTWPALPATLDSAFEDPQTKRVFFFSGRQMWVYTGKTVLGPRSLDKLGLGPEVTHVSGLLPRRLGKALLFSKGRVWRFDLKSQKVDPQSVIRVDKEFSGVPWNSHDIFQYQDKAYFCHGKFFWRVSFQNEVNKVDHEVNQVDDVGYVTYDLLQCP

SEQ ID NO: 11 - DNA, 2387 bp, Homo sapiens
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttaaaaaaaaaaaaaaaaaaaaaaaaaaaaaaaaaaaaaaaaaaaaaaaaaaaa

SEQ ID NO: 12 - DNA, 3185 bp, Mus musculus
ctcaccatgagtccctggcagcccctgctcctggctctcctggctttcggctgcagctctgctgccccttaccagcgccagccgacttttgtggtcttccccaaagacctgaaaacctccaacctcacggacacccagctggcagaggcatacttgtaccgctatggttacacccgggccgcccagatgatgggagagaagcagtctctacggccggctttgctgatgcttcagaagcagctctccctgccccagactggtgagctggacagccagacactaaaggccattcgaacaccacgctgtggtgtcccagacgtgggtcgattccaaaccttcaaaggcctcaagtgggaccatcataacatcacatactggatccaaaactactctgaagacttgccgcgagacatgatcgatgacgccttcgcgcgcgccttcgcggtgtggggcgaggtggcacccctcaccttcacccgcgtgtacggacccgaagcggacattgtcatccagtttggtgtcgcggagcacggagacgggtatcccttcgacggcaaggacggccttctggcacacgcctttccccctggcgccggcgttcagggagatgcccatttcgacgacgacgagttgtggtcgctgggcaaaggcgtcgtgatccccacttactatggaaactcaaatggtgccccatgtcactttcccttcaccttcgagggacgctcctattcggcctgcaccacagacggccgcaacgacggcacgccttggtgtagcacaacagctgactacgataaggacggcaaatttggtttctgccctagtgagagactctacacggagcacggcaacggagaaggcaaaccctgtgtgttcccgttcatctttgagggccgctcctactctgcctgcaccactaaaggccgctcggatggttaccgctggtgcgccaccacagccaactatgaccaggataaactgtatggcttctgccctacccgagtggacgcgaccgtagttgggggcaactcggcaggagagctgtgcgtcttccccttcgtcttcctgggcaagcagtactcttcctgtaccagcgacggccgcagggatgggcgcctctggtgtgcgaccacatcgaacttcgacactgacaagaagtggggtttctgtccagaccaagggtacagcctgttcctggtggcagcgcacgagttcggccatgcactgggcttagatcattccagcgtgccggaagcgctcatgtacccgctgtatagctacctcgagggcttccctctgaataaagacgacatagacggcatccagtatctgtatggtcgtggctctaagcctgacccaaggcctccagccaccaccacaactgaaccacagccgacagcacctcccactatgtgtcccactatacctcccacggcctatcccacagtgggccccacggttggccctacaggcgccccctcacctggccccacaagcagcccgtcacctggccctacaggcgccccctcacctggccctacagcgccccctactgcgggctcttctgaggcctctacagagtctttgagtccggcagacaatccttgcaatgtggatgtttttgatgctattgctgagatccagggcgctctgcatttcttcaaggacggttggtactggaagttcctgaatcatagaggaagcccattacagggccccttccttactgcccgcacgtggccagccctgcctgcaacgctggactccgcctttgaggatccgcagaccaagagggttttcttcttctctggacgtcaaatgtgggtgtacacaggcaagaccgtgctgggccccaggagtctggataagttgggtctaggcccagaggtaacccacgtcagcgggcttctcccgcgtcgtctcgggaaggctctgctgttcagcaaggggcgtgtctggagattcgacttgaagtctcagaaggtggatccccagagcgtcattcgcgtggataaggagttctctggtgtgccctggaactcacacgacatcttccagtaccaagacaaagcctatttctgccatggcaaattcttctggcgtgtgagtttccaaaatgaggtgaacaaggtggaccatgaggtgaaccaggtggacgacgtgggctacgtgacctacgacctcctgcagtgcccttgaactagggctccttctttgcttcaaccgtgcagtgcaagtctctagagaccaccaccaccaccaccacacacaaaccccatccgagggaaaggtgctagctggccaggtacagactggtgatctcttctagagactgggaaggagtggaggcaggcagggctctctctgcccaccgtcctttcttgttggactgtttctaataaacacggatccccaaccttttccagctactttagtcaatcagcttatctgtagttgcagatgcatccgagcaagaagacaactttgtagggtggattctgaccttttatttttgtgtggcgtctgagaattgaatcagctggcttttgtgacaggcacttcaccggctaaaccacctctcccgactccagcccttttatttattatgtatgaggttatgttcacatgcatgtatttaacccacagaatgcttactgtgtgtcgggcgcggctccaaccgctgcataaatattaaggtattcagttgcccctactggaaggtattatgtaactatttctctcttacattggagaacaccaccgagctatccactcatcaaacatttattgagagcatccctagggagccaggctctctactgggcgttagggacagaaatgttggttcttccttcaaggattgctcagagattctccgtgtcctgtaaatctgctgaaaccagaccccagactcctctctctcccgagagtccaactcactcactgtggttgctggcagctgcagcatgcgtatacagcatgtgtgctagagaggtagagggggtctgtgcgttatggttcaggtcagactgtgtcctccaggtgagatgacccctcagctggaactgatccaggaaggataaccaagtgtcttcctggcagtcttttttaaataaatgaataaatgaatatttacttaaaaaaaaaaaaaaaaaaaaaaaaaaaaaaaaaaaaaaaaaaaaaaa

SEQ ID NO: 13 - protein, 115 aa, Artificial Sequence
  Description of Artificial Sequence Synthetic polypeptide
EVQLLESGGGLVQPGGSLRLSCAASGFTFSLYSMNWVRQAPGKGLEWVSSIYSSGGSTLYADSVKGRFTISRDNSKNTLYLQMNSLRAEDTAVYYCARGRAFDIWGQGTMVTVSS

SEQ ID NO: 14 - protein, 108 aa, Artificial Sequence
  Description of Artificial Sequence Synthetic polypeptide
DIQMTQSPSSLSAFVGDKVTITCRASQSVGTYLNWYQQKAGKAPELLIYATSNLRSGVPSRFSGSGSGTDFTLTINTLQPEDFATYYCQQSYSIPRFTFGPGTKVDIK

SEQ ID NO: 15 - protein, 115 aa, Artificial Sequence; identical to SEQ ID NO: 13
  Description of Artificial Sequence Synthetic polypeptide

SEQ ID NO: 16 - protein, 108 aa, Artificial Sequence
  Description of Artificial Sequence Synthetic polypeptide
DIQMTQSPSSLSASVGDRVTITCRASQSVGTYLNWYQQKPGKAPKLLIYATSNLRSGVPSRFSGSGSGTDFTLTISSLQPEDFATYYCQQSYSIPRFTFGPGTKVDIK

SEQ ID NO: 17 - protein, 120 aa, Artificial Sequence
  Description of Artificial Sequence Synthetic polypeptide
EVQLLESGGGLVQPGGSLRLSCAASGFTFSVYGMVWVRQAPGKGLEWVSVISSSGGSTWYADSVKGRFTISRDNSKNTLYLQMNSLRAEDTALYYCARPFSRRYGVFDYWGQGTLVTVSS

SEQ ID NO: 18 - protein, 107 aa, Artificial Sequence
  Description of Artificial Sequence Synthetic polypeptide
DIQMTQSPSSLSASVGDRVTITCRASQGIRNFLAWYQQKPGKVPKLLVFGASALQSGVPSRFSGSGSGTDFTLTISGLQPEDVATYYCQKYNGVPLTFGGGTKVEIK

SEQ ID NO: 19 - protein, 120 aa, Artificial Sequence
  Description of Artificial Sequence Synthetic polypeptide
EVQLLESGGGLVQPGGSLRLSCAASGFTFSVYGMVWVRQAPGKGLEWVSVISSSGGSTWYADSVKGRFTISRDNSKNTLYLQMNSLRAEDTAVYYCARPFSRRYGVFDYWGQGTLVTVSS

SEQ ID NO: 20 - protein, 107 aa, Artificial Sequence
  Description of Artificial Sequence Synthetic polypeptide
DIQMTQSPSSLSASVGDRVTITCRASQGIRNFLAWYQQKPGKVPKLLIYGASALQSGVPSRFSGSGSGTDFTLTISSLQPEDVATYYCQKYNGVPLTFGGGTKVEIK

SEQ ID NO: 21 - protein, 124 aa, Artificial Sequence
  Description of Artificial Sequence Synthetic polypeptide
FYSHSAQSELTQPPSASAAPGQRVTISCSGSSSNIGSNTVTWYQKLPGTAPKLLIYNNYERPSGVPARFSGSKSGTSASLAISGLQSEDEADYYCATWDDSLIANYVFGSGTKVTVLGQPKANP

SEQ ID NO: 22 - protein, 161 aa, Artificial Sequence
  Description of Artificial Sequence Synthetic polypeptide
MKKLLFAIPLVVPFVAQPAMAEVQLLESGGGLVQPGGSLRLSCAASGFTFSPYLMNWVRQAPGKGLEWVSSIYSSGGGTGYADSVKGRFTISRDNSKNTLYLQMNSLRAEDTAVYYCARIYHSSSGPFYGMDVWGQGTTVTVSSASTKGPSVFPLAPSSKS

SEQ ID NO: 23 - protein, 14 aa, Artificial Sequence
  Description of Artificial Sequence Synthetic peptide
TGTSSDVGGYNYVS

SEQ ID NO: 24 - protein, 7 aa, Artificial Sequence
  Description of Artificial Sequence Synthetic peptide
DVSKRPS

SEQ ID NO: 25 - protein, 10 aa, Artificial Sequence
  Description of Artificial Sequence Synthetic peptide
CSYAGSYTLV

SEQ ID NO: 26 - protein, 5 aa, Artificial Sequence
  Description of Artificial Sequence Synthetic peptide
TYQMV

SEQ ID NO: 27 - protein, 17 aa, Artificial Sequence
  Description of Artificial Sequence Synthetic peptide
VIYPSGGPTVYADSVKG

SEQ ID NO: 28 - protein, 15 aa, Artificial Sequence
  Description of Artificial Sequence Synthetic peptide
GEDYYDSSGPGAFDI

SEQ ID NO: 29 - protein, 110 aa, Artificial Sequence
  Description of Artificial Sequence Synthetic polypeptide
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTLVFGGGTKLTVL

SEQ ID NO: 30 - protein, 106 aa, Artificial Sequence
  Description of Artificial Sequence Synthetic polypeptide
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 31 - protein, 110 aa, Artificial Sequence
  Description of Artificial Sequence Synthetic polypeptide
QYELTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTLVFGGGTKLTVL

SEQ ID NO: 32 - protein, 124 aa, Artificial Sequence
  Description of Artificial Sequence Synthetic polypeptide
EVQLLESGGGLVQPGGSLRLSCAASGFTFSTYQMVWVRQAPGKGLEWVSVIYPSGGPTVYADSVKGRFTISRDNSKNTLYLQMNSLRAEDTAVYYCARGEDYYDSSGPGAFDIWGQGTMVTVSS

SEQ ID NO: 33 - protein, 110 aa, Artificial Sequence; identical to SEQ ID NO: 31
  Description of Artificial Sequence Synthetic polypeptide

SEQ ID NO: 34 - DNA, 330 bp, Artificial Sequence
  Description of Artificial Sequence Synthetic polynucleotide
cagtacgaattgactcagcctcgctcagtgtccgggtctcctggacagtcagtcaccatctcctgcactggaaccagcagtgatgttggtggttataactatgtctcctggtaccaacagcacccaggcaaagcccccaaactcatgatttatgatgtcagtaagcggccctcaggggtccctgatcgcttctctggctccaagtctggcaacacggcctccctgaccatctctgggctccaggctgaggatgaggctgattattactgctgctcatatgcaggcagctacactttggtgttcggcggagggaccaagctgaccgtccta

SEQ ID NO: 35 - protein, 124 aa, Artificial Sequence; identical to SEQ ID NO: 32
  Description of Artificial Sequence Synthetic polypeptide

SEQ ID NO: 36 - DNA, 372 bp, Artificial Sequence
  Description of Artificial Sequence Synthetic polynucleotide
gaagttcaattgttagagtctggtggcggtcttgttcagcctggtggttctttacgtctttcttgcgctgcttccggattcactttctctacttaccagatggtttgggttcgccaagctcctggtaaaggtttggagtgggtttctgttatctatccttctggtggccctactgtttatgctgactccgttaaaggtcgcttcactatctctagagacaactctaagaatactctctacttgcagatgaacagcttaagggctgaggacacggccgtgtattactgtgcgagaggggaggactactatgatagtagtggcccgggggcttttgatatctggggccaagggacaatggtcaccgtctcaagc

SEQ ID NO: 37 - protein, 110 aa, Artificial Sequence; identical to SEQ ID NO: 29
  Description of Artificial Sequence Synthetic polypeptide

SEQ ID NO: 38 - protein, 124 aa, Artificial Sequence; identical to SEQ ID NO: 32
  Description of Artificial Sequence Synthetic polypeptide

SEQ ID NO: 39 - DNA, 330 bp, Artificial Sequence
  Description of Artificial Sequence Synthetic polynucleotide
cagagcgccctgacccagcccagaagcgtgtccggcagcccaggccagagcgtgaccatcagctgcaccggcaccagcagcgacgtgggcggctacaactacgtgtcctggtatcagcagcaccccggcaaggcccccaagctgatgatctacgacgtgtccaagaggcccagcggcgtgcccgacaggttcagcggcagcaagagcggcaacaccgccagcctgaccatctccggactgcaggccgaggacgaggccgactactactgctgcagctacgccggcagctacaccctggtgttcggcggagggaccaagctgaccgtgctg

SEQ ID NO: 40 - protein, 110 aa, Artificial Sequence; identical to SEQ ID NO: 29
  Description of Artificial Sequence Synthetic polypeptide

SEQ ID NO: 41 - DNA, 372 bp, Artificial Sequence
  Description of Artificial Sequence Synthetic polynucleotide
gaggtgcaattgctggaaagcggcggaggactggtgcagccaggcggcagcctgaggctgtcctgcgccgccagcggcttcaccttcagcacctaccagatggtgtgggtgcgccaggccccaggcaagggcctggaatgggtgtccgtgatctaccccagcggcggacccaccgtgtacgccgacagcgtgaagggcaggttcaccatcagcagggacaacagcaagaacaccctgtacctgcagatgaacagcctgagggccgaggacaccgccgtgtactactgcgccaggggcgaggactactacgacagcagcggcccaggcgccttcgacatctggggccagggcacaatggtgaccgtgtccagc

SEQ ID NO: 42 - protein, 124 aa, Artificial Sequence; identical to SEQ ID NO: 32
  Description of Artificial Sequence Synthetic polypeptide

SEQ ID NO: 43 - DNA, 654 bp, Artificial Sequence
  Description of Artificial Sequence Synthetic polynucleotide
cagagcgccctgacccagcccagaagcgtgtccggcagcccaggccagagcgtgaccatcagctgcaccggcaccagcagcgacgtgggcggctacaactacgtgtcctggtatcagcagcaccccggcaaggcccccaagctgatgatctacgacgtgtccaagaggcccagcggcgtgcccgacaggttcagcggcagcaagagcggcaacaccgccagcctgaccatctccggactgcaggccgaggacgaggccgactactactgctgcagctacgccggcagctacaccctggtgttcggcggagggaccaagctgaccgtgctgggccagcccaaggctgcccccagcgtgaccctgttcccccccagcagcgaggaactgcaggccaacaaggccacactggtgtgcctgatcagcgacttctacccaggcgccgtgaccgtggcctggaaggccgacagcagccccgtgaaggccggcgtggagacaaccacccccagcaagcagagcaacaacaagtacgccgccagcagctacctgagcctgacccccgagcagtggaagtcccacaggtcctacagctgccaggtgacccacgagggcagcaccgtggagaaaaccgtggcccccaccgagtgtagctgatga

SEQ ID NO: 44 - DNA, 1365 bp, Artificial Sequence
  Description of Artificial Sequence Synthetic polynucleotide
gaggtgcaattgctggaaagcggcggaggactggtgcagccaggcggcagcctgaggctgtcctgcgccgccagcggcttcaccttcagcacctaccagatggtgtgggtgcgccaggccccaggcaagggcctggaatgggtgtccgtgatctaccccagcggcggacccaccgtgtacgccgacagcgtgaagggcaggttcaccatcagcagggacaacagcaagaacaccctgtacctgcagatgaacagcctgagggccgaggacaccgccgtgtactactgcgccaggggcgaggactactacgacagcagcggcccaggcgccttcgacatctggggccagggcacaatggtgaccgtgtccagcgccagcaccaagggccccagcgtgttcccgctagcaccttcctccaagtccacctctggcggcaccgccgctctgggctgcctggtgaaggactacttccctgagcctgtgaccgtgagctggaactctggcgccctgacctccggcgtgcataccttccctgccgtgctgcagtcctccggcctgtactccctgtcctccgtggtgacagtgccttcctcctccctgggcacccagacctacatctgcaacgtgaaccacaagccttccaacaccaaggtggacaagcgggtggagcctaagtcctgcgacaagacccacacctgccctccctgccctgcccctgagctgctgggcggaccctccgtgttcctgttccctcctaagcctaaggacaccctgatgatctcccggacccctgaggtgacctgcgtggtggtggacgtgtcccacgaggacccagaggtgaagtttaattggtatgtggacggcgtggaggtccacaacgccaagaccaagcctcgggaggaacagtacaactccacctaccgggtggtgtccgtgctgaccgtgctgcaccaggactggctgaacggcaaggaatacaagtgcaaagtctccaacaaggccctgcctgcccccatcgagaaaaccatctccaaggccaagggccagcctcgcgagcctcaggtgtacaccctgcctcctagccgggaggaaatgaccaagaaccaggtgtccctgacctgtctggtgaagggcttctacccttccgatatcgccgtggagtgggagtccaacggccagcctgagaacaactacaagaccacccctcctgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgagccctggcaagtga

SEQ ID NO: 45 - protein, 218 aa, Artificial Sequence
  Description of Artificial Sequence Synthetic polypeptide
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSSS

SEQ ID NO: 46 - protein, 455 aa, Artificial Sequence
  Description of Artificial Sequence Synthetic polypeptide
EVQLLESGGGLVQPGGSLRLSCAASGFTFSTYQMVWVRQAPGKGLEWVSVIYPSGGPTVYADSVKGRFTISRDNSKNTLYLQMNSLRAEDTAVYYCARGEDYYDSSGPGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKS

SEQ ID NO: 47 - DNA, 26740 bp, Homo sapiens
cgctcagggaaggcgggtgcgcgcctgcggggcggagatgggcagggggcggtgcgtgggtcccagtctgcagttaagggggcaggagtggcgctgctcacctctggtgccaaagggcggcgcagcggctgccgagctcggccctggaggcggcgagaacatggtgcgcaggttcttggtgaccctccggattcggcgcgcgtgcggcccgccgcgagtgagggttttcgtggttcacatcccgcggctcacgggggagtgggcagcgccaggggcgcccgccgctgtggccctcgtgctgatgctactgaggagccagcgtctagggcagcagccgcttcctagaagaccaggtaggaaaggccctcgaaaagtccggggcgcattcggcacttgttttgtttggtgtgatttcgtaaacagataattcgtctctagcccaggctaggaggaggaggagataaccgccggtggaggcttccccattcgggttacaacgacttagacatgtggttctcgcagtaccattgaacctggacctcccttcacacagcccctcaatcgtgggaaactgaggcgaacagagcttctaaacccacctcagaagtcagtgagtcccgaatatcctgggtgggaatgactaagacacacacacacacacacacacacacacacacacacacacacacacagtaggaaaggtgtatttcaagcacactttctttctccttggggagaattattgctaaccatctaagttttctggaggcggccttttttctccccagcctcccggcggggtcaccctctcccaccttccaggagagtggaggacccgtgagatacggggcacgcaggcagcgacttcctgaaatgctaacaaggatcgtaggatcagttactgctgcgaggagcaagcacttgcttcttgggggagttttgcagccaacagggaaatgggctttctttgtgagttagaggtagaggtccggcggcctgagtgattgaaactgctcgggacaatgctcgtatgtttagcaaacgacagaactgtagaactgttcctgagaaatcccaactgatagtattttagtcatctcagacgacagttagcacagtttaaaaatgaggcctacttcttgaaaaacagaatccaaggtagttttgtcctcacattgacaaatgttgacacagccagtgtaatttcctataaccaggaaaactgaaagaatatatgtacagttaaaatatgtacaatgctaattaaaacttgtgtaataagtctaaaagtaatttaatgaggcttcacttttatgaccgtccttgtggtatgcttcgccaggaatatatagcttcaaaaagcaaaggccagcggaggggtaattatttttttactgcaatgttaattgtctctttgacatggaaatataaacctgttaaaactatcagtgtttaatttagtgtctcaatttctattagcaaaaatttataatctataggataaatgcacattttattttttacttttcatattatgcaagttaatttttttaatttagtcaaaggagcttataaaggatttcagggcctgttgctggatttgattttaattcattttgaaacattgacaagaccctggttgttgttttttttaacagtggtttatccgtatcagcaaaagtttagccactgtgaccggtaactgtatgaatatagttcttaatattattgtctatataaaaatatttattactctagttaatattattctatataaaatcattttgtttaaattattaagttgcctctgaaaatctgtagtaacaaagtagaacatgtcaatgtatataaatgccataattatgtattttttagtttaggcctataaaacataacattgtggtgattttaagttagagaaaatattttatagtatgttaatgtatatgcatgaaatgcaaaaatatttaaatgataggttcattgaaatagatcattttttgttatttaggtataaatcaattttcaggacgtatgtgaaaagcgcaatcttcaggaagtttctcaagatagaacacagcttggatagaatgtcttgaaatatatgcaattttccaatttcatatgtaaaatgatatacataatataaaatctagcggtgttaattataatgatatgtaattatatatttcacattaatatattttatgcccatggctatattgatttgggaatatatatggatactaattatgttaggattcatacaattccttgagaggcacaagtgctaaaaattacttgtatgaattatttaatatcattgcaaataagatgttattttaactttttttaagtttctgcaaatatgtttattatgactttttatttttatatgattggaaatacatatactaaaattccacgttaccagtttcttaaccacagaaacctgaaaaattgccatagttgatttgttacttctaccttggtgcatttacaaaatagtcatatttttattatgaagttaaatattcatttgtttatagctacttcagaaggctcaggttatttttttctttaatagcacagagtcctctcaaggtaagcactgtgcagttagtataaaccattattccccatgtgtacatgattcacagtttgtattgtgttccaagtgaaccatagccctttcagaaatcaagacttatattcattttacttctttgagtactcttgaattttagaaagtccattatgatcctaaggtagcaacaacatagcctattaccgtctatgatggtttacagatctattattccacgttagttcatcactatcaactaccatgatagagttaagctaaaccattttcccaacatatgaaaaactcctattactaaagtgatacaaatggtatcaaaaatacttttttatagcaaggttcaacagtgggcccagtgcttttacactttttcaaaagtccttggagaaacagagaaaatctcacttgccttctgtactaaaacattctaggccgaactaaaactgaaacttcatagtagaacactgtaggccaggggtgttcaatcttttaccttccctgggccacataggaagaagaagaattgtcttgggacacacattaaatacactaacactaacaatagctgatgagcttaaaaaaaaaaaaaaactcatgatactttaagaaagtgtatgaatttgtgttgggcagcattcaaaaccatcctgggctgcatgtgaccctcgggcccacaggttggacaagcttgccctagctcctccatctgctgcaaagcccagcctgatacaaaaaccaacgtgataaaaagtttttgtggtgctttattttggcagtttaagttatataaacaatgggtacagtttcattttctaaatataaaatttttacattgaatatgaatttttaagacaaattatctgaattctgattctcatatacctaactactaatatcttctctatttgttgcccaatgagattaatccacctcttaaacacttcaccatcaagaaaaacaattttgtattttaaaatgaacccatccactttcattcagctattttatattcaggcatcatcctaaggaaagaaaggttctgacaaagattaatacagatggataagtagtagcaagaaatcaaaaactgcataaaattctagcaataaagtgttaaattatggtacagttacattctggatcatcaggtatctgaagaatatttcatagactgttaaatgattgcattataaagtcaggtttttttaaagcaagattccaaacagtaaacagtttctctctctctctctctctctctctctctctctctctctcaccaaattagttataatggtttccgcaggatgagaggggttgggaaaaagtttggtgatatttattttcttcgtttcacttttgagttttccaaagtgctatgaccatcatcagtaaaatatacatttccaaagcctttgacacacggtaacagtcctacacagtggatgaactaagagcttctctacccttagatgggtagggagggaggaaagacaaggaaactgagttgtttaagtgtcatacacgagaacgtggctttaaggtctgggaaaacctgcgagggctgtgacgtcagactgtgaaatgcacgctatgtccattcaccaagacgttccattttaaaacccataaatccgtagctatacctgtttccaaggtgcctcgtgttaggcctctggtcacagcacttggcgcccttcttgggatctcttctctccgcccccactaccccaccccacaagcacactctagtcccctccaatcaatttcaggcaggtctcgccgcctccggagccacgctgggggtgcaagggccctggacccgaaagagcgcccgcccggcgacaagagatgagatgcacgctgctcctccactcctcagcccccaccatcctcctcctggatcctaacttccccactctctcaattcctagagacgctgcggatcccagaggcttaactggcagctggaacgaggtcctccaacaagaatttagacgctaggtccaattatcactccaccgcgcgcactttccgcaggagcgatgtgatccgttatcataactgcggacctggggttccacgtggaagacgattgggatttcactggccgcggtgggggtgggagcagacagagtctgagtggggttagtggactcgagacgaaaggcaggacatgacagaaggcaactctgggtcacctctccagcttggaactggctaggccttgttttggaggggatgggtagatgaaaagtgagtcagggttacccggaggaaccacggggaaagtgcgcttctgagactcttgacagccatttcgttcccttccaagccagatggagacccaagagtgttgaaaggccacgacttccctcagtttctccatctgggggtgcaggatggtatagagagtggcccgtagtatttttccagtgacgatgtctctccattgttttcttcttatattgcagctttccccatgtttgaaaattttcttttcaaatgaaatcattgattagaataaaaaaaagtaagtagctattaaaacaagatcaatttccatgacagtaagccaaccgatggagaaaaccttgggaattaataaatgaaggatttgtttggtagatgataaaaggtccttttaaagggtctgactcttcctagaaaaacccaccaacttgggaccgcaacagatttaccatatcctaattcatgctattttaatgtgtattcagcaaacccacatgtgtttacaattgtcgaagctaccaaatgtcaatagcgttttttttctatttgttgaatgtgaatctcttgtacgaagccatataaacagaagaaattacaggaatgattttaaatcacatacaaaaccaatagtattgctagaggagagttagtcaaggacggcattatgaagaaagtgagggagaatttccaaagagcagaacgatagggcttggtggaccaaagaacgtttccatctaaagggaatggcaaatacttagagtctctgaacccactgaatcttggactatttaactaatatttgtagttccagatatagcacagtgccttgtacatagtggtatttttaaaaatatagtgcctcgtagattttttttcaacttttatttaggaggagagggcacatgtgcaggttaattacaaaggtatattgcaccatgctgaggtttcgagtacgactgaatctgtcactcaagtagtgagcacagtacccacagtaggtagtatttcagccctcgctcattccctttctcctccatctagtagtccccaatgtctattgttctcatatttatgtccaattagcatttgttttttaaaaagggtggttgaagaaattctcagtgcttgtcagtgtctctcagtgcattcatttaattcatgagccctggaatgatggtttcatttgggcagaactctacaatcaaaaagaagtaataaaagggaaaaaaaagtgaaagccatcaactacaggattgaaattcccaaagcatcagaggtcctttcaaaaaatagtatgttgatttttaatttttatgacttattggctttgttcatgaaaatataaacatgttatcacaaaggattttttaattcaactatttctcagttttctctttcaccttcaaaataaaatatcataaattatttaaatggttgtgaaggcagtaggatttttttaagagagaaaagttttatagaggttcagaattacatgaacaaagacatgtaatctcttaagcaaattgaaactaataaaatcgtacaatcaaggtaacgtaaataaaaaagcctctgctttcttaattgaattatgtgagtaactagaaattttaaaagtatggcaaaggttaacaacagcattattacctgggctgcctttaaaaatacatatttctggggttcacgttcagaaaatttgattcagatttgctgtgggtcccagaaatctgcattttaaataaacacttgaaggagatactaatacaagtggcccattgggacacaatttgacaaatatgaccaattttactttttaaaccttatttctgcttctttatctttgaattgaggtccaggattttaggtaagattttaagtttagagtcagtttactggatcccagggaggagagtctgagtaatcagtggaggagttatttcaccaaatgaaggagaccctttattattatgtgaccctttgtatgaattggaaaagaatgtcttgtagataccacatttttacagtcagaacatagtttgagagaaaaaaatataacaagatatatttgtgttttaaagcttacagaaccagacagaaaatttccacataagctatataagatacgttgtctttttaaaacactatatacacttctttctgttcgtgcaggatgaatggatctctctctctctctctctctctctgtgtgtgtgtgtgtgtgtgtgtgtgtgtgtgtgtgttgtaataaggggtttctttcattttatgatccagaccaggctcgtaataaacatgacaacctaaaattatgtaaaaaagaaaaatcaaagcacaagtgtttcacaggtttaacttatgcttatctaagatcagggcaagattgcaggaaaatgtagccataacagaataaagcatttatggacaaaatgatgggtctttatgtctctgtaaaagcacagtgatggggggggaaatatagatgaaaaatgtaagctaaaaagtaacaattataagaaaaactaaaatatcatgcctttcaaatgatcatttttctgcttttaagctaaaatttgtctaatattacaccagtgactttgctgatgtattaggaaaaagcttgttttgctttcttttctcgagtgccaccattttcttgctctcattctctttcaggctgccagatcatctgactcagcaattgtataactctctcacccaatttaaagaaacagcagctgtctagagaacaatgactcccccagttgaacatctaattgttaaatgtccaacatcggacactttgaattttactccatgcaatttacatgctgaatagttgaagttgaatatattatatttaacatttaatttttaaaagcttattgaaactttcttcctaaatcacatggtaaagttattgttttcttcaaaaacaattaggaggagcttaacaataataggacacttcaacttccattatctaatttaattatcacaatatccttatgttttcaatgtttcattttttcattttgtagatctggagactgaggctcagataggttgcatggcctaccaaaagtcattgactagtaattcatatatagttgaacttggttgcccatggagtgctataaatatgtatatggtttcagttccatctcttttagttaactattattttgaaagtcgcttaacccctttgggcctctactatactcaagcatcagccgtataagtcacagtaaatatttattggttgaaaggaggttaacatctttcaaaaatttattttttgaccaaaataaaaccagtgaaaaattctcatatgactgtacatataaattacttattcctaccttaatttaaaagcaataagtgggatacctattcaccagcacaggaaccacttgaagcgtgcagttgaaagattactttctttagcattcacatgacctgtgagcagattctatttcttttgcttattagctgtcatggtaccagaatgaagtatgagaaactctcagtgctttcatgttctcatctgtaaacctgagaccctatggtagtcccgtaataagaggtagataaaatagtatgtgtgaagagtcactgtaaacttttacacagtgtacgtttgtcagttattatagtgcctaattaaactatgcccttaagaaagcacattagttttttacagtaaatacctacttcattataatttttcagtgtagctagaaatttctaaactccactttaaaaatatacatatcataataaaaatatatttatgtattcagactcctggtatgttccaaggtgttaggtaaaatcagtgtaaatttgcatacttttaaattcacatctgtacagaagatctatatggtggcctttagggtatacctctaagctattctagtattcataatcattaaagagatattaagcagtgtttgtgaacccctgttttctaagacaggaaatcaaggtagctttagaaaactggaaaaaaagttattagtctatctatctaataacccagaataataatttccaaaggaatcactgaagataactggatttttaattccttcagaatggttgtcacagtctgaatatctgaatcaacagttttgaccaaaacaattttctaaaaattctttagtataaaaaattatgtgtgtgtgtgtctgtgtgatgaaaggaatgataggcagaaacattactgtcatccttacgacattcaaaatgcctaccttggagggtgaccttcagttatttttatgcaaatgtgaagaagttatttagaagtaggatatcaaagagtaacacaaaatacactaaatagtatgctttcttaaggctaaattgacttgggggttttaaatcagtacagagtaaacatacagtatattctgttatcattgcctttttgaaaaattaattatggaagttatcatcttaaccgtaacaacacaaaagataaaactctaccctcaacccagagactcaaaggaaaacatgagtggaaatgttaaatctgtatgtgaaaagtgctaaaacatgaataggaagcagttacttatttaatcaaagttgattatatttcatcaagaagttgattcccttgagtggagttgaatcacatatcaggtgaagaatgtgatttggggaagaatggtctaacacaagaaaattttcttgcaatctttaataatatcagaggggagattggcttcagaactctcctaagttcaggaaaggacacagaaaattgaacataacagtaagactatagagtcccaagaaagcaagctacttttaaaggatagttttttagaggggcaaaagggggacaaccattctccatttgatgagaaaagcttccatgtagatggtgcccctgaaattagagtatcctaaaccagtgttaaacctatcagtgaaacatgaatattaaacctccactcccagtagtgaaaaccgaatacattattatttatctgtgactttcaacattatctcagaactctaacagcacatgcgtacatcagcagcataagcagaaatgagatattatatatgcttgtgttagcaattaaaaaggacagcatatttgagaggggaaaatctgtcctatcaagaatgaaaaagagggaggttaggaaaagtagtttagagaaagtaaattttgcaattcctcagttttaactgtagtttctccattgtaccttccacttgaaatgcactccaagcagtggaggtgggtagcaatgaatgcagaggaaacactgaacacagtgacactctccagtgtcacttctcatgatttaatgaggggttttttttggaaattcttctgtcataacatgggaaactttgttacaaagaagctgttttttcagagggttagaattcagaggtagcatcataccttttagaagagaatttgcttgttgaaaccacagatacctgctagaatgtacaggaattaatgaaaaattactcaaaaggacatttattttgatgacctaaatgaataacttcatagtaaatgtcatatatattctcaaaaaattaaaaagcaccatttattgagagcctaccgtgcacccggatttttatatatctgacattctttattcctcacagtaaccttatggggtaaattttattttccccactttgtgaggtgaggaaataaaggctcagaaagtttacataacttattcaagcccacagagctggtaaatgagaggtcagttctatctgagtttaaagactaggcttgtcccacttgcatatgtgtcatttccaaaattatgattaaggatatggttggcatttcccgccacccacattaagtccaattaagtagctgtggccatagaaagaatggagaatggagagaggaactgacttcaacagctacagcaaacatttattagctgagtaaccatagctacatagttcctcaatatgtaccactcctccattttgttatctataaatcaaaatggtggctttttaaaaagcagttttacaatatattcaagagccttctaccctttgaaaaactgcaatactatttttagtagcaattagaaacaccttaaatatctgacaacagggacatcattaagtaaattataactttttccagtgggatgtgttacagctgttaaaagtagcatttatgaagtgtttttggagaagtttggaaaatgctgtaataagttagaaaaagctcatttcaaaattgcataatattcacaatgtaaagattaagcaaagaaaaaggaagaagtatttcaaaatgttaataattattgctttgtgtggggtagtttttcattttctatgtgcagctaattccttaattatttttaaatatgtgagctttaatcaggaaagcaaatcattcaaaaatgaggggactgaattaagtgactttcaggggactttgcgtgtctttgagttccaaatttctatcactatgtattactactgaagaataatcatagaagcacagtagtttctgaaaatggagagtcagtaatcttggcccaggttttgcaacttgctctaaagcagagtcctcaaagaaaaggaagcattgatgagttgtccacaatgtactggataaattatcattaggaaaacatattgtagtagggagagtgaggacctctcaaacagaactgagaaccttaagtttgaacttttcttttccttattacttaagcactctgagcttttttttttgtctgcattatgaagaaagaataatactctctatcccatgggacagctgtggaattataaattacacatataaaactgcttgatgcttgtcacatagctgggggttgaaaaaatgatagccattattttcttggcaacttttaatgaattttttattatctctatttctttctgcctatctcctctaattatgtttattacttattttgttcctcaggatgaggtcaattctcaatatctgtgctgtacataatatacatatataccaaatatgtgcatatagtatgtacatacatacatactgtgctaatcttttagtgttctcagctgatcaaatagctacaaatagatataagtaattcgccacaagtaatttatcaacataaaaaaaatttacaaaaaagttaaggaataattgtctccatgagctgcaaagatccctcatttcacaagagtacaccctagagatattttaatagtaaatttctcacatagatttaaaatcacatttgttttgcacataatttagaaaagatacctgctatataataagtaatatacttttaagtttccttcaaaatattcttgggaagatgataataggtactgctaattctatacccagttaacattttggaaactaaggttgaaaattgtgacttaactataattatgcattaaatctacaacacatcaaagaattttgcattttgtactccttactaagatccagtttgagtaggaagataaattttacagtaattctgaatgagggaagttggcacagagtttctaaaagagtaccttccttatagcaaatactaaataattgtgctatattgaatttaattaaatagagaatagtaaaagggagaaagaaacatccaatgttttgaaacttctagagatctactcccagggacacattgttttttcttagcaaatctgtttggaggtctgctctactttctcagaggtctccctttcatgctgaagctatcttttttccttgtggaacataagtaattaaataccttgcaattatttacctaagaaagtgtttctttcccgtttaaaatgctcttaccacccacattggactcgattatcagaatttttatccggggcagcttcaggagcactttggcacttcggggctaaaccacaatctgtttttacatgtttgtgattatacccgttttgtagatcaagacattgaagctagtaaaaaaaaaaaaaagtcattttttcagggtaacaaagtaggtggtagaactaggacagggactctaatttccttacattattgcttttctaaattaaagggatgcatggaattattcctccattgcctttgccttcaaataattatctattgcacccaacatcctattctagaactcatctatgaaggcttaacacagctgtacctgggagctccattacagggcatatatctcgctctcataagctacttcctaaggaattctctttaattatgggagcttttccagactctgaaatctttttttcctggtaacacaagtgtgaggtgtcatttatcagaatgcatcaccccagtcttccctcctcaaatgattactgtaggctccactcaagagctcatcccagttcaagaccaccttcctcctccagagaagcaaatatatatatacacgtatatatatatatacacgtatatatatatatacacgtatatatatatatacacgtatatatatatatacacgtatatatatatatacacgtatatatatacacgtatatatatatatacacgtatatatatatacacgtatatatatatatacacgtatatatatatacacgtatatatatatatacacgtatatatatatacgtgtatatatatatatacattttttttttttgagacggagtctcgctctgttgcccaggctggagtgcagtggcgcgatctcggctcactgcaagctccgccccccgggttcacgccattctccttcctcagcctccggagtagctgggactacaggtgcccgccacctcgcctggctaattttttgtatctttagtagagatggggtttcaccgtgttacctaggatggtctagatctcctgacctcgtgatccgcccgcctcggcctcccaaagtgctgggattacaggcgtgagccaccgcgcctggcagagaagcaaatatattgatggttgttaccaatacatgctcttgactaagaaaccttctttcttaattaatattgacaactttaagccgagtgcctgacatatattaggtactcagttactctttttcaactaaagttatgaatgatgattctaataaaagtaacttatttgtctactagttttattatgtttatttaattcattagaaaggccatggacatagtacaaaattcaaacaatataaatcatggaatgtgaaaagtaagtcacatgcccatcccagttcttcatttccttacctcacaggtaacagcttttcctgtatctccccagagatattctatgtatattttgtttttaacaccaagctatatttaaaacaattatctttaataataatgttaatattgaaactggtaaagaaatatgtgtgtattatctcacctcaagcgtaaacaatagaacaagagagagcccattttgaaaattatggacaatgaatctagaaataatctcaaaagattttgcagtcaaaaaatagttcattagatacatgagaactgtcacttggtctcagtgtagagctattgcctcaactccctttattttcctaacaaaatcatcttgcttatcccatgaaatacgtgcatattgccaatcctacaatgccgcatcagaaccagaacccaactctggaacactaccttctcaagtatctttctgtctctttatggtaatatgttgaattaatattcacatctattatgactagtctttgatttgtagggttgctgaagtagtagcaccactgcagggctttctttagtttaaagaaagtaatcaggtgtccctactgtgtcatgatctccaccctcagctgggttctccagtctggttttaaagaacaaaacaaaaggcttctctgtctgagtcttactcaacccatcctctctactcataagaggtattccaaacctttacgattctcaaacttcctaaccgaccatcttattttcactctgcaaacaagctaacctcctcattcatagaaggaagtgcctcaacttcctccccgttctgaccttttctccctcccaaatctatgtatctcttgtgacaaaatctataaccaccgctgtactttgagttctatttcttcattatttttgagggacctcaagtcctcaaaaatatcctatcttgcctgtgtacttaacttttcttttattcttttctaactttcccttctcttcacttggcacttgcccttccaggtatatgtgtgctcaggtctcctccaccttccatctgcctcacttcatggcatagggccttgaactatcacaaccaagctatgaaagagtagtcaacgcagtgtccccacttccttgccatcccattatcctagtttttcttttggctctctgaggagtccttcacaggctggttttcaggaataagtctaaatgaatcactttcagttttcctaaacttctatgcctttgcacatcctcttacctctgcctagaatatctttctccttcttttccatctttaaactctcacatcattcttcaagactgggatcagctctcagcatccggaagcctttgcctactagagacaaatgagaatgagtttggtcaccttttcattttcttgtatcattctgtgctttattttgctcttctaagagcgttacatgcttcatttaatccctaaacaactgtttgaggcaagtacagttattatcctaatcatgcaaatgagaaaacagaggcccagacatgttgagtaactttgataaaagttaaagaaccaataagtggaacagttgaggtttgaaccctggcagtctgactgtagagatactatgtttgacctactcccctctgcccccaccccatgtctgcccttagtttctgagcttgttgaatgaatgaacaggtggtagtctttttttgttataagactgatcagaattaagacaggtttaaatttcacgtgtagaattttcaaaactgcaaaggcagtgcaaatctaaaaaaagaatggcattctcaggaaagaggaaaagtaagtgtgagaataataataacaataaccaacaaactttagtaaatttagtaaatgtagtaaatttttacattaaaagcttttggacatacattatcatattttatggccacatgaaatatattataatcccattttgcacataggaaatctgagactggcataaggagcacagagatccaggactttatattttcattcttctaggattttgcacctcaggtcgatatgtatgagtaaactgggagtataatgggctctttaacagaaaaactaggaaagttttcccactattattaattatttacataatatttttttaattttattattatttatactttaagttttagagtacatgtgcacaatgtgcaggtttgttacatatgtatacatgtgccatgttggtgtgctgcacccatcaactcatcatttagcattaggtatatctcctaatgctatccctcccccctcccccctacataagatttataatggataatggacttcaatttctagagcaaaatggccccacccaaggatgccataatccttccagagctctactgcaagatatgagatatacatatctaaaacttgttcttggtatttccaaagcagtcaacttttacacctgtttataatgcatccaaatgttgtttttatatggttgcatctcccatcttcttcaccaatagctatatatatttttcacaagagctgaaagagttcttgatgtaggaatccatggtagagtttcagagaaatccctgaattcactgaaagttttatctagaaatacatgtgcaagtgaacacatcttttttaaaaaaaatcattacctactttcttttttgagaagaaggtatttatttcaacagactcttgaaggagcctactcttcccactctcccacccccattaagaaccactgtaggccgggcacgatggctcatgcctgtaatcccagcactttgggaggctaaggtgggtggatcacctgaggtcaggagttcgagacaagcctagccaacatagtgaaaccccgtctctactaataatacaaaaattagctgggtatggcagcatgtgcctgtaatcccagctactcgggaggctgaggcaggagaattgctcgaacccgggaggcggaggttgcagtgaaccgagagagatcgtgcggtgccatttcactccagcctgggcaacagagcgaaactccatctcaaaaaaacacacaaaacaaacaaacaaaaagaaagaaccattgtattagtgatggaaatgtgttccctccctcccatcctggcaaccactttcttcctcctccatcataaaatatcttaaactaaactaaaataattttatttatcgatagtttgaattttccctatcattgctacacagctaattgagaggtaccccgaggaaaatataaatggtacagtaatgcattgtagattttaataacatacttgacatcccaaattgttttcattggcttcattttaaaaactacatgttttaaaatcaagcagacactaaaagtacaagatatactgggtctacaaggtttaagtcaaccagggattgaaatataacttttaaacagagctggattatccagtaggcagattaagcatgtgcttaaggcatcagcaaagtctgagcaatccattttttaaaacgtagtacatgtttttgataagcttaaaaagtagtagtcacaggaaaaattagaacttttacctccttgcgcttgttatactctttagtgctgtttaacttttctttgtaagtgagggtggtggagggtgcccataatcttttcagggagtaagttcttcttggtctttctttctttctttctttctttttttcttgagaccaagtttcgctcttgtctcccaggctggagtgcaatggcgcgatctcggctcactgcaacctccgccttctcctgggttcaagcgattctcctacatcagcctccgagtagctgggattacaggcatgcgccaccaagccccgctaattttgtattttttagtagagacagggtttcgccatgttggtcaggcttgtctcgaactcctggcctcaggtgatccgcctgtctcggcctcccagaatgctgggattatagacgtgagccaccgcatccggactttccttttatgtaatagtgataattctatccaaagcatttttttttttttttttgagtcggagtctcattctgtcacccaggctggagggtggtggcgcgatctcggcttactgcaacctctgcctcccgggttcaagcgattctcctgcctcagcctcctgagtagctggaattacacacgtgcgccaccatggccagctaatttttgtatttttagtagagacggggtgtcaccattttggccaagctggcctcgaactcctgacctcaggtgatctgcccgcctcggcttcccaaagtgctgggattacaggtgtgagccaccgcgtcctgctccaaagcattttctttctatgcctcaaaacaagattgcaagccagtcctcaaagcggataattcaagagctaacaggtattagcttaggatgtgtggcactgttcttaaggcttatatgtattaatacatcatttaaactcacaacaacccctataaagcagggggcactcatattcccttccccctttataattacgaaaaatgcaaggtattttcagtaggaaagagaaatgtgagaagtgtgaaggagacaggacagtatttgaagctggtctttggatcactgtgcaactctgcttctagaacactgagcactttttctggtctaggaattatgactttgagaatggagtccgtccttccaatgactccctccccattttcctatctgcctacaggcagaattctcccccgtccgtattaaataaacctcatcttttcagagtctgctcttataccaggcaatgtacacgtctgagaaacccttgccccagacagccgttttacacgcaggaggggaaggggaggggaaggagagagcagtccgactctccaaaaggaatcctttgaactagggtttctgacttagtgaaccccgcgctcctgaaaatcaagggttgagggggtagggggacactttctagtcgtacaggtgatttcgattctcggtggggctctcacaactaggaaagaatagttttgctttttcttatgattaaaagaagaagccatactttccctatgacaccaaacaccccgattcaatttggcagttaggaaggttgtatcgcggaggaaggaaacggggcgggggcggatttctttttaacagagtgaacgcactcaaacacgcctttgctggcaggcgggggagcgcggctgggagcagggaggccggagggcggtgtggggggcaggtggggaggagcccagtcctccttccttgccaacgctggctctggcgagggctgcttccggctggtgcccccgggggagacccaacctggggcgacttcaggggtgccacattcgctaagtgctcggagttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagcatggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtgggtagagggtctgcagcgggagcaggggatggcgggcgactctggaggacgaagtttgcaggggaattggaatcaggtagcgcttcgattctccggaaaaaggggaggcttcctggggagttttcagaaggggtttgtaatcacagacctcctcctggcgacgccctgggggcttgggaagccaaggaagaggaatgaggagccacgcgcgtacagatctctcgaatgctgagaagatctgaaggggggaacatatttgtattagatggaagtatgctctttatcagatacaaaatttacgaacgtttgggataaaaagggagtcttaaagaaatgtaagatgtgctgggactacttagcctccaattcacagatacctggatggagcttatctttcttactaggagggattatcagtggaaatctgtggtgtatgttggaataaatatcgaatataaattttgatcgaaattattcagaagcggccgggcgcggtgcctcacgccttgtaatcccttcactttgggagatcaaggcggggggaatcacctgaggtcgggagttcgagaccagcctggccaacaggtgaaacctcgcctctactaaaaatacaaaaagtagccgggggtggtggcaggcgcctgtaatcccagctactcgggaggttgaggcaggagaatcgcttgaacccgggaggctgaggttgtagtgaacagcgagatggagccacttcactccagcctgggtgacagagtgagactttgtcgaaagaaagaaagagagaaagagagagagaaaaattattcagaagcaactacatattgtgtttatttttaactgagtagggcaaataaatatatgtttgctgtaggaacttaggaaataatgagccacattcatgtgatcattccagaggtaatatgtagttaccattttgggaatatctgctaacatttttgctcttttactatctttagcttacttgatatagtttatttgtgataagagttttcaattcctcatttttgaacagaggtgtttctcctctccctactcctgttttgtgagggagttaggggaggatttaaaagtaattaatacatgggtaacttagcatctctaaaattttgccaacagcttgaacccgggagtttggctttgtagtcctacaatatcttagaagagaccttatttgtttaaaaacaaaaaggaaaaagaaaagtggatagttttgacaatttttaatggagacgggagaagaacatgtagaaaaggggaaatgatgttggcttagaatcctaactacattggtgtttaatataggaacatttatttatataacattttaaagtactaaattcatattagtatattatcaaatggatatattatcaaatgggtttaagcatcctacacattttaattcaattgattcattttctttttgctttggatttctatcatgatttaaatatttacatatgggttactttttagatttttcatactatgaaatataagaaaaacctttaaggctagttttatgaccaagacgaaggacttcattgaatacacaaaacaataaatatactgcaacattttgtctttctttttgtagctgcaatttggtttgcttatactttctctttgtctctttgaaaactgagtcagtttcactttctcaggacaggatttaataaccataatataatttagtataattccttgatttaggcaaattatgcaatttgtgtttagtatgaaatgtacctaaaaataagtaactcctctttaacaccaccatcctcaaactaatataacaaataacagttatcctaaaataaattgtctacttccaccatgcagcactcaaattttaaggttgctatgactgcagacagtattttaaaattcctctctggaaatggctttgtttccaagatgatttaggaaccaaagaggtgaccatctcttgtttaatgaactctcaaatcataaacctgggaagtgttttagtttcctactgctgctgttacaaattatcacaaatgtgttagctaaaacaaacacaaaattattattttacagttctagagatcagaagtcaaaaatgggtccacaaggtttcattccttttggaaactctaaggggcaatctgtttccttgtcttttccagcttctagtgaccatcaaattccttggctcatggtctctgtattttctctgtggcctgtgcttccattcttgtatcttctctctgactgtgaccctctaataaaaacacttggggttatgttgggcccaccctgaaaattctggataatctccctcaagaccattaattaaatcacatctgcaaagcctcttttgccacataagttaatgtattaaaagtttttgaggattaggacatagacattgggggtgggggggcattattcagcctaccacaggaaggaattttagggttaattaaactagccttcttattttatacttgaagaaattgaagttttggaattggagagcattatgctaaatgaaataagccaaacacagaaagacaaatatcacatgttctcacttatctgtgaaatataaaacaattacattcttagcagtaaagagtagaatggtggttactagagctggggggtgggaggaatggggagatggtaatcaagatataaagcctcagttaagatgggaggaataagtttgattgttttttttgagatgtgtttcatagcatgatgaatatagctaaatagtaaatcccaaatgctctcatttgacaaaaatgtcaaatatttgagatgatggataggttacttagcttgacttaataattccccattgtgttcaaagatcataacttcatattgtaccacataaatatatacaactgtactatcccaatatataattttaaaactaatataatgaaaaagaaattgaagttcaacattcccagaagctaagtgtaacttaaaagttttgtgagaatttgttttaacaaacaaacaagttttctctttttaacaattaccacattctgcgcttggatatacagcagtgaacaaaaaaaaaaaaaaaaatctccaggcctaacataatttcaggaagaaatttcagtagttgtatctcaggggaaatacaggaagttagcctggagtaaaagtcagtctgtccctgcccctttgctattttgcccgtgcctcacagtgctctctgcctgtgacgacagctccgcagaagttcggaggatataatggaattcattgtgtactgaagaatggatagagaactcaagaaggaaattggaaactggaagcaaatgtaggggtaattagacacctggggcttgtgtgggggtctgcttggcggtgagggggctctacacaagcttcctttccgtcatgccggcccccaccctggctctgaccattctgttctctctggcaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcaggtgaggactgatgatctgagaatttgtaccctgagagcttccaaagctcagagcattcattttccagcacagaaagttcagcccgggagaccagtctccggtcttgcctcagctcacgcgccaatcggtgggacggcctgagtctccctatcgccctgccccgccagggcggcaaatgggaaataatcccgaaatggacttgcgcacgtgaaagcccattttgtacattatacttcccaaagcataccaccacccaaacacctaccctctgctagttcaaggcctagactgcggagcaatgaagactcaagaggctagaggtctagtgccccctcttcctccaaactagggccagttgcatccacttaccaggtctgtttcctcatttgcataccaagctggctggaccaacctcaggatttccaaacccaattgtgcgtggcatcatctggagatctctcgatctcggctcttctgcacaactcaactaatctgaccctcctcagctaatctgaccctccgctttatgcggtagagttttccagagctgccccagggggttctggggacatcaggaccaagacttcgctgaccctggcagtctgtgcaccggagttggctcctttccctcttaaacttgtgcaagagatcgctgagcgatgaaggtagaattatggtcctccttgcccttgcctttcctttttgtgatctcaaagcatcctccctccgcccccattccatggccccagttccctactcccacagctgtctgctgaaactgccaacattactcaattgtttctggggggaggaacatttttttttgaaacaaaatagatatatgaaacagtacacgggaattaacacgaatatttaaggtaaaacatgaccttgaagattatgaaatccatcttattttggcccagaacgggggcattgggctccttgggccataggggagctggggaggacagggtgaagagttagctctaagccctctgcttggagatgctgtaaatacagaacgcaaaatcaccttcgaagttaaagacgcgaagttcttctttactcggcccctcctcccctcccccccgccaattccctccagttacagctagcatccaggtcccgggaggtgaagaaggagacttcggctccagttacagctagcatccgggtcccgatttagaaggagctgccaattacagcgcggttccagggctgagcaaaaagcctgaggagccaagtgggagagggagtaaaactactgaattgggccacaagcaaatgaataaactgaacgactcttaaccaaacctaatatatttaatccaaacacacaagtctttcatttcttccctcctcccttccttctcttactccccaacaccccctcttcaagcacaattaattatatggttagattctactgcgtgatcagccctgttctaggtggtgggcacgccaaggtgaatgagaccaaacaagagtcttgccctcatggggtttacatttggagacagagtcgatctgttgcccaacctggagtgcagtggcgcgatcacagctcactgcagcctcaaactccctggctcaaggggttctcccacctgagcctcccgactagctgggaccacaggtgcacgccacgacgcctgggtttgtttgtttgtttaatagagacgaaggtctcaccatgttatctgggctcaagcgatcatcccccctcctcctcctaaagtactgggattacagtcccaagctatcttgcccgacctgggaaacagacgttaaggaagataacaatctattttcagagagcgagtttataaaaccaatgcaatgggtaaatatgaagtgtgaataggaggagaagctaaagagtggtcggagaatctaatgcaagctacgggagaaagaaactcaagtgcaaatgctgcctcaggaataaacgtaaaaagagactttcaagtgcaaatgctccctcaggaataaaataatcttgagactctcaagtgtaaatgctgcctcgggagaaccgaacggcgagctggagcccatacgcaacgagattagagaggaaggcagaagccagagcacatgaataaatgagcatccattttgtttcagaaatgatcggaaaccatttgtgggtttgtagaagcaggcatgcgtagggaagctacgggattccgccgaggagcgccagagcctgaggcgccctttggttatcgcaagctggctggctcactccgcaccaggtgcaaaagatgcctggggatgcgggaagggaaaggccacatcttcacgccttcgcgcctggcattgtgagcaaccactgagactcattatataacactcgttttcttcttgcaaccctgcgggccgcgcggtcgcgctttctctgccctccgccgggtggacctggagcgcttgagcggtcggcgcgcctggagcagccaggcgggcagtggactagctgctggaccagggaggtgtgggagagcggtggcggcgggtacatgcacgtgaagccattgcgagaactttatccataagtatttcaatgccggtagggacggcaagagaggagggcgggatgtgccacacatctttgacctcaggtttctaacgcctgttttctttctgccctctgcagacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaaatgtcctgccttttaacgtagatatatgccttcccccactaccgtaaatgtccatttatatcattttttatatattcttataaaaatgtaaaaaagaaaaacaccgcttctgccttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggcagaaccaaagctcaaataaaaataaaataattttcattcattcactca

SEQ ID NO: 48 - DNA, 1464 bp, Homo sapiens
cgagggctgcttccggctggtgcccccgggggagacccaacctggggcgacttcaggggtgccacattcgctaagtgctcggagttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagcatggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagaaatgatcggaaaccatttgtgggtttgtagaagcaggcatgcgtagggaagctacgggattccgccgaggagcgccagagcctgaggcgccctttggttatcgcaagctggctggctcactccgcaccaggtgcaaaagatgcctggggatgcgggaagggaaaggccacatcttcacgccttcgcgcctggcattacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaaatgtcctgccttttaacgtagatatatgccttcccccactaccgtaaatgtccatttatatcattttttatatattcttataaaaatgtaaaaaagaaaaacaccgcttctgccttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggcagaaccaaagctcaaataaaaataaaataattttcattcattcactcaaaaaaaaaaaaaa

SEQ ID NO: 49 - DNA, 1235 bp, Homo sapiens
atggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtgggtagagggtctgcagcgggagcaggggatggcgggcgactctggaggacgaagtttgcaggggaattggaatcaggtagcgcttcgattctccggaaaaaggggaggcttcctggggagttttcagaaggggtttgtaatcacagacctcctcctggcgacgccctgggggcttgggaagccaaggaagaggaatgaggagccacgcgcgtacagatctctcgaatgctgagaagatctgaaggggggaacatatttgtattagatggaagtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaaatgtcctgccttttaacgtagatatatgccttcccccactaccgtaaatgtccatttatatcattttttatatattcttataaaaatgtaaaaaagaaaaacaccgcttctgccttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggcagaaccaaagctcaaataaaaataaaataattttcattcattcactcaaaaaaaaaaaaaa

SEQ ID NO: 50 - DNA, 1267 bp, Homo sapiens
cgagggctgcttccggctggtgcccccgggggagacccaacctggggcgacttcaggggtgccacattcgctaagtgctcggagttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagcatggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaaatgtcctgccttttaacgtagatatatgccttcccccactaccgtaaatgtccatttatatcattttttatatattcttataaaaatgtaaaaaagaaaaacaccgcttctgccttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggcagaaccaaagctcaaataaaaataaaataattttcattcattcactcaaaaaaaaaaaaaa

SEQ ID NO: 51 - DNA, 1164 bp, Homo sapiens
cgctcagggaaggcgggtgcgcgcctgcggggcggagatgggcagggggcggtgcgtgggtcccagtctgcagttaagggggcaggagtggcgctgctcacctctggtgccaaagggcggcgcagcggctgccgagctcggccctggaggcggcgagaacatggtgcgcaggttcttggtgaccctccggattcggcgcgcgtgcggcccgccgcgagtgagggttttcgtggttcacatcccgcggctcacgggggagtgggcagcgccaggggcgcccgccgctgtggccctcgtgctgatgctactgaggagccagcgtctagggcagcagccgcttcctagaagaccaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaaatgtcctgccttttaacgtagatatatgccttcccccactaccgtaaatgtccatttatatcattttttatatattcttataaaaatgtaaaaaagaaaaacaccgcttctgccttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggcagaaccaaagctcaaataaaaataaaataattttcattcattcactcaaaaaaaaaaaaaa

SEQ ID NO: 52 - protein, 156 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 53 - protein, 167 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSEMIGNHLWVCRSRHA

SEQ ID NO: 54 - protein, 132 aa, Homo sapiens
MVRRFLVTLRIRRACGPPRVRVFVVHIPRLTGEWAAPGAPAAVALVLMLLRSQRLGQQPLPRRPGHDDGQRPSGGAAAAPRRGAQLRRPRHSHPTRARRCPGGLPGHAGGAAPGRGAAGRARCLGPSARGPG

SEQ ID NO: 55 - protein, 116 aa, Homo sapiens
MEPAAGSSMEPSADWLATAAARGRVEEVRALLEAGALPNAPNSYGRRPIQVGRGSAAGAGDGGRLWRTKFAGELESGSASILRKKGRLPGEFSEGVCNHRPPPGDALGAWEAKEEE